(19) United States
(12) Patent Application Publication
(10) Pub. No.: US 2026/0207649 A1
(43) Pub. Date: Jul. 23, 2026

(54) LSD1 INHIBITOR AND PRMT6 INHIBITOR FOR USE IN THE TREATMENT OF A DISEASE ASSOCIATED WITH GAIN-OF-FUNCTION OF ANDROGEN RECEPTOR (AR) AND/OR WITH OVEREXPRESSION OF AN AR COACTIVATOR

(71) Applicants: FONDAZIONE TELETHON ETS(30%), Roma (IT); FONDAZIONE ISTITUTO ITALIANO DI TECNOLOGIA(10%), Genova (IT); GENETHON(7%), Evry-Courcouronnes (FR); UNIVERSITA' DEGLI STUDI DI TRENTO(20%), Trento (IT); INSTITUT NATIONAL DE LA SANTÉ ET DE LA RECHERCHE MÉDICALE (INSERM)(7%), Paris (FR); UNIVERSITÉ D'ÉVRY-VAL D'ESSONNE(6%), Evry (FR); UNIVERSITA' DEGLI STUDI DI PADOVA(20%), Padova (IT)

(72) Inventors: Maria Pennuto, Padova (IT); Manuela Basso, Trento (IT); Giuseppe Ronzitti, Evry-Courcouronnes (FR); Andrea Contestabile, Genova (IT)

(73) Assignees: FONDAZIONE TELETHON ETS (30%), ROMA (IT); FONDAZIONE ISTITUTO ITALIANO DI TECNOLOGIA (10%), Genova (IT); GENETHON (7%), Evry-Courcouronnes (FR); UNIVERSITA' DEGLI STUDI DI TRENTO (20%), Trento (IT); INSTITUT NATIONAL DE LA SANTÉ ET DE LA RECHERCHE MÉDICALE (INSERM) (7%), Paris (FR); UNIVERSITÉ D'ÉVRY-VAL D'ESSONNE (6%), Evry (FR); UNIVERSITA' DEGLI STUDI DI PADOVA (20%), Padova (IT)

(21) Appl. No.: 19/141,348

(22) PCT Filed: Dec. 20, 2023
(86) PCT No.: PCT/IB2023/063001

§ 371 Date: Jun. 20, 2025

(30) Foreign Application Priority Data
Dec. 22, 2022 (IT) 102022000026595

(51) Int. Cl. A61K 31/7105; A61P 21/00; A61P 35/00; C12N 15/113

(52) U.S. Cl. CPC A61K 31/7105; A61P 21/00; A61P 35/00; C12N 15/1137; C12N 2310/141

(57) ABSTRACT

The present invention is directed to a therapeutic agent for use in the treatment of a disease associated with gain-of-function of androgen receptor (AR) and/or with overexpression of an AR coactivator, such as Kennedy disease or cancer. The therapeutic agent of the invention comprises at least one inhibitor of an androgen receptor (AR) coactivator, selected from protein arginine methyltransferase 6 (PRMT6) inhibitor, lysine specific demethylase 1 (LSD1) inhibitor, or combinations thereof.

### FIELD OF THE INVENTION

The present invention is directed to a therapeutic agent for use in the treatment of a disease associated with gain-of-function (GOF) of androgen receptor (AR) and/or with overexpression of an AR coactivator, preferably a disease associated with toxic GOF of AR, with or without overexpression of an AR coactivator, such as Kennedy disease or cancer, preferably urological cancers, such as prostate, bladder and renal cancers.

The therapeutic agent of the invention comprises at least one inhibitor of at least one androgen receptor (AR) coactivator, selected from an inhibitor of protein arginine methyltransferase 6 (PRMT6), an inhibitor of lysine specific demethylase 1 (LSD1), or combinations thereof (“combo therapy”).

### BACKGROUND

AR is a transcription factor activated by androgens. To properly exert its pleiotropic functions in different tissues, AR interacts with transcriptional co-regulators. In the unliganded state, AR mainly, but not exclusively, localizes to the cytosol and associates with heat shock proteins. Upon androgen binding, AR dissociates from heat shock proteins and translocates to the nucleus, where it binds to the androgen-responsive elements (AREs) present in promoter or enhancer elements of its target genes, to regulate gene expression. Transcriptional regulation acts in both a sequential and combinatorial manner to reorganize chromatin. Central to this dynamic reorganization is the modification of core histones. The N-terminal tails of histones are subject to various covalent modifications such as acetylation, phosphorylation, ubiquitination and methylation by specific chromatin-modifying enzymes, many of which are AR cofactors.

Cofactors (also called coregulators) are recruited for the proper activity of steroid receptors. Almost 300 AR coregulators are known, which work as coactivators or corepressors of AR and are essential for the regulation of gene expression. PolyQ expansions alter the native functions of AR, resulting in aberrant gene expression in both motor neurons and muscle cells.

Spinobulbar muscular atrophy (SBMA), also known as Kennedy's disease, is an X-linked late-onset neuromuscular disease caused by microsatellite expansions (≥38 repeats) of a glutamine (Q)-encoding CAG triplet tandem repeat in exon 1 of the androgen receptor (AR) gene, resulting in the production of an AR with an aberrantly elongated polyglutamine (polyQ) tract¹. SBMA belongs to the family of diseases caused by polyQ expansions, which includes Huntington's disease (HD), dentatorubral-pallidoluysian atrophy, and six types of spinocerebellar ataxia (SCA)². SBMA affects 2-5/100,000 people worldwide and is characterized by the selective degeneration of lower motor neurons^{3,4}. Emerging research also demonstrates primary involvement of peripheral tissues such as skeletal muscle⁵. The clinical features of SBMA include late-onset progressive muscle weakness, fatigue and fasciculations, dysphagia, endocrine dysfunction and mild-to-moderate metabolic syndrome, and, in some individuals cardiac dysfunction. Phenotypes are attributed largely to toxic gain-of-function (GOF) of polyQ-expanded AR. Yet mild signs of androgen insensitivity and endocrine abnormalities also implicate partial AR loss-of-function (LOF) in this condition¹. A necessary step to toxicity is the binding of AR with its natural ligands, testosterone and its more potent derivative dihydrotestosterone (DHT)⁸. As such, SBMA is unique among polyQ diseases for its sex specificity (in humans as well as in fly and mouse models): males develop severe symptoms, while females develop mild or no symptoms even if homozygous for the mutation.

No therapy exists to cure SBMA or delay disease onset and progression. Although the androgen-dependent nature of the disease and experimental evidence support chemical castration as a therapeutic strategy for SBMA, clinical trials based on this approach show benefits only in a subset of patients 3. Moreover, chronic androgen ablation may enhance symptoms associated with androgen deprivation, spanning from muscle atrophy and weakness to metabolic alterations and depression.

This poses a limit to clinical approaches to silence the disease protein, an aspect that is particularly relevant for chronic, slow-progressive diseases, such as SBMA, that require long-term treatment regimes. This aspect is important for SBMA for three reasons: i) any therapy is more likely to work if started at puberty, concomitant with or before appearance of symptoms; ii) any therapy suppressing mutant AR will enhance AR LOF, an aspect that cannot be neglected in an X-linked disease affecting male subjects; and iii) SBMA patients show signs of androgen insensitivity syndrome and, for a therapy that will be administered for the entire life of the patient, this aspect should be taken into account in the study design for new treatment. Enhancement of AR LOF is likely to exacerbate sexual dysfunction, metabolic syndrome and diabetes, depression and muscle atrophy. Thus, alternative strategies are necessary to improve clinical outcomes. In particular, there is a critical need for novel therapeutic agents that can be administered chronically with minimal side effects, i.e., a therapy that preserves AR nuclear functions while abolishing the toxic gain of functions.

It is known that dysregulated expression of AR co-regulators contributes to the onset and progression of prostate cancer and other types of hormone-dependent cancers, such as bladder, liver, and kidney cancers. In prostate cancer, overexpression of about 30% of AR co-regulators results from aberrant AR signalling.

Previous transcriptomic analysis identified significant upregulation of protein arginine methyltransferase 6 (PRMT6) in the skeletal muscle of a mouse model of SBMA. PRMT6 is a general transcriptional co-repressor yet also is a co-activator of AR. PRMT6 expression is often upregulated also in hormone-dependent cancers, such as prostate cancer⁴ and breast cancer, and in mouse models of metabolic syndrome, diabetes, and insulin resistance—symptoms that also are present in about 50% of SBMA patients.

Similar to PRMT6, lysine-specific demethylase 1 (LSD1, AOF2, or KDM1A) is a transcriptional co-activator of AR⁶ and is upregulated in prostate cancer⁷.

Notably, both LSD1 and PRMT6 have a steroid hormone binding motif, LXXLL (where L is leucine and X is any amino acid). Mechanistically, both PRMT6 and LSD1 bind the AF-2 surface of AR located in AR ligand-binding domain through their steroid hormone binding motif, LXXLL.

PRMT6 and LSD1 are both required to form a functional complex with AR for the full response to androgens. Androgen binding induces numerous post-translational modifications on AR, including phosphorylation and lysine and arginine methylation.

Transcription co-regulators do not directly bind DNA but rather often possess enzymatic activity and exert their function by modifying histone proteins, resulting in changes in chromatin structure, transcription factor accessibility to enhancers and promoters, transcription factor and co-factor recruitment at the poised genes, and interaction with the preinitiation complex. LSD1 catalyses the demethylation of H3K4me1/me2.

Transcription co-factors also post-translationally target nonhistone proteins involved in gene transcription. PRMT6 methylates and transactivates oestrogen receptor alpha, CREB-regulated transcriptional co-activator 2, DNA topoisomerase 3B3, p16^{IKK4a}, p21^{CIP1}, DNA polymerase beta, and high mobility group Ala. LSD1 also targets non-histone proteins, including Forkhead Box A1, DNA methyltransferase 1, p53, hypoxia-inducible factor alpha, oestrogen-related receptor alpha, and E2F.

LSD1 and PRMT6 synergistically transactivate AR itself.

The inventors found that in pathological condition, such as SBMA or cancer, a feedforward mechanism occurs, whereby the toxic GOF of AR enhances the expression of its own positive co-regulators, PRMT6 and LSD1, which in turn further boost AR function, leading to pathological dysregulation of expression of AR target genes.

Surprisingly, the inventors have provided proof-of-principle that selectively targeting those AR co-regulators, that are aberrantly overexpressed in AR-associated diseases, is a valuable therapeutic strategy for patients, without enhancing AR LOF. Accordingly, the inventors have surprisingly found that therapeutic efficacy is synergistically improved by providing a therapeutic agent that targets both PRMT6 and LSD1 AR co-regulators (“combo therapy”).

### BRIEF DESCRIPTION OF THE INVENTION

The limitations of the prior art are overcome by the present invention, providing novel therapeutic agents for the treatment of diseases associated with GOF of AR and/or with overexpression of an AR coactivator, preferably of a disease associated with toxic GOF of AR, with or without overexpression of an AR coactivator, that act through inhibition of overexpressed AR coactivators.

The present invention is in particular directed to a therapeutic agent as set forth by the present claims. Preferably the therapeutic agent comprises or consists of a genetic silencer of an AR coactivator, or a small molecule inhibitor of an AR coactivator, or combinations thereof.

More preferably, the therapeutic agent comprises RNA interfering molecules (such as artificial microRNAs), antisense oligonucleotides, pharmacological agents (small molecules), or genome editing agents, targeting PRMT6 and/or LSD1, or combinations thereof.

Preferably, the present invention is directed to a therapeutic agent comprising a combination of at least one inhibitor of PRMT6 and at least one inhibitor of LSD1.

According to preferred aspects, the present invention is directed to a therapeutic agent comprising at least one inhibitor of PRMT6 and/or at least one inhibitor of LSD1 for use in the treatment of diseases associated with gain-of-function of androgen receptor (AR) and/or with overexpression of an AR coactivator.

According to preferred aspects, the present invention is then directed also to a method of treating a disease associated with gain-of-function of AR and/or with overexpression of an AR coactivator, comprising administering to a subject in need thereof a therapeutic agent comprising at least one inhibitor of lysine specific demethylase 1 (LSD1) and/or at least one inhibitor of protein arginine methyltransferase 6 (PRMT6), preferably said therapeutic agent comprising at least one inhibitor of LSD1 and at least one inhibitor of PRMT6.

Moreover, the present invention is directed to novel genetic silencers targeting PRMT6 and/or LSD1 transcripts.

The invention is best understood from the following detailed description when read in conjunction with the accompanying drawings.

### BRIEF DESCRIPTION OF FIGURES

FIG. 1 shows the early and persistent androgen-dependent overexpression of LSD1 and PRMT6 in the skeletal muscle of SBMA mice and patients.
a) RT-PCR analysis of Lsd1 and Prmt6 transcript levels in the indicated tissues of male WT and AR100Q mice at pre-symptomatic stage (4 weeks), disease onset (8 weeks), and late stage (12 weeks) (n=3-5 mice/genotype).
b) Western blots of LSD1 and PRMT6 levels in the quadriceps muscle of 12-week-old WT and AR100Q mice (n=4 mice/genotype). Quantification is shown at the bottom.
c) RT-PCR analysis of Lsd1 and Prmt6 transcript levels in the quadriceps muscle of female WT and AR100Q mice (n=4 mice/genotype).
d) RT-PCR analysis of Lsd1 and Prmt6 transcript levels in the EDL muscle of sham-operated and surgically castrated 8-week-old male AR100Q mice (n=3-4 mice/genotype).
e) RT-PCR analysis of Lsd1 and Prmt6 transcript levels in the quadriceps muscle of 24-week-old male knock-in mice expressing AR113Q (n=3-4 mice/genotype).
f) RT-PCR analysis of Lsd1 and Prmt6 transcript levels in C2C12 myoblasts stably transduced with empty lentiviral vector (mock) or vector expressing AR100Q and differentiated to myotubes in presence of DHT (10 nM, 10 days, n=3-5 biological replicates).
g) RT-PCR analysis of LSD1 and PRMT6 transcript levels of quadriceps muscle biopsies of control (CTR) subjects and SBMA patients (n=5 participants/genotype).
h) Chromatin-immunoprecipitation (ChIP) assays in C2C12 myoblasts expressing AR24Q and AR100Q and treated with vehicle and DHT (10 nM, 12 h). one experiment representative of three technical replicates is shown. LSD1 and PRMT6 were detected with specific antibodies, and calnexin (CNX) was used as loading control. Graphs show mean±SEM; Student t-test (b, e, f, g, h), or two-way ANOVA followed by Tukey HSD tests (a, c, d), * p≤0.05; ** p≤0.01; *** p≤0.001.

FIG. 2. shows that LSD1 is a co-activator of polyQ-expanded AR.
a) Proximity ligation assays (PLA) in motor neuron-derived MN1 cells expressing AR24Q (left graphs) and AR100Q (right graphs) and treated with vehicle or DHT (10 nM, 16 h). Graphs show quantification of nuclei from three biological replicates (AR24Q AR/LSD1 vehicle: n=26; AR24Q AR/LSD1 DHT: n=43; AR24Q AR/PRMT6 vehicle: n=20; AR24Q AR/PRMT6 DHT: n=8; AR100Q AR/LSD1 vehicle: n=76; AR100Q AR/LSD1 DHT: n=61; AR100Q AR/PRMT6 vehicle: n=66; AR100Q AR/PRMT6 DHT: n=43).
b) Transcriptional assays in HEK293T cells expressing AR24Q or AR65Q alone (mock, i.e., empty vector) or together with LSD1 and treated with vehicle or DHT (10 nM, 16 h, n=4 biological replicates).
c) Transcriptional assays in MN1 cells expressing AR65Q alone (mock) or together with the indicated LSD1 isoforms and treated with vehicle or DHT (10 nM, 16 h, n=3 biological replicates).
d) (Top) Western blots of LSD1 levels in HEK293T cells expressing Cas9 with or without specific guides to silence LSD1; quantification is shown at the bottom (n=7 biological replicates). (Bottom) Transcriptional assays in Cas9 and g1, g2, g2+3 cells expressing AR24Q or AR65Q and treated with DHT (10 nM, 16 h, n=3 biological replicates).
e) (Top) Western blots of LSD1 levels in MN1 cells expressing Cas9 with or without a specific guide (g4) to silence Lsd1; quantification is shown at the bottom (n=5 biological replicates). (Bottom) Transcriptional assays in Cas9 and g4 cells expressing AR24Q or AR100Q and treated with DHT (10 nM, 16 h, n=3 biological replicates).

LSD1, PRMT6, and AR were detected with specific antibodies, and beta-tubulin (β-Tub) was used as loading control. Graphs show mean±SEM; one-way (d) or two-way (b, c, e Western blot) ANOVA followed by Tukey HSD tests, or student t test (a, e transcriptional assays). * p≤0.05; ** p≤0.01; *** p≤0.001.

FIG. 3 shows that LSD1 requires the AR AF-2 surface and its catalytic activity to transactivate AR.
a) Scheme of AR and LSD1 modular domains and specific motifs. Numbers refer to AR NM_000044 and LSD1 NM_001009999. NTD, amino-terminal domain; DBD, DNA-binding domain; LBD, ligand-binding domain; AOD, amine oxidase domain; CTD, carboxy-terminal domain.
b) Transcriptional assay in HEK293T cells expressing AR55Q or the AF-2 mutant, AR55Q-E897K, alone (mock) or with LSD1. Cells were treated with DHT (10 nM, 16 h, n=3 biological replicates).
c) (Left) Transcriptional assay in HEK293T cells expressing AR65Q alone (mock) and with either LSD1 or LSD1-LXXAA. Cells were treated with DHT (10 nM, 16 h, n=3-6 biological replicates). (Right) Western blotting analysis of LSD1 and LSD1-LXXAA expression in HEK293T cells. Shown is one representative image of 3 biological replicates.
d) Transcriptional assay in HEK293T cells expressing AR55Q alone (mock) and with either LSD1 or the catalytic inactive mutant, LSD1-K685A. Cells were treated with DHT (10 nM, 16 h, n=3 biological replicates).
e) Transcriptional assay in HEK293T cells expressing AR65Q treated with DHT only or together with SP-2509 (100 nM) and TCP (10 μM) for 16 h (n=3 biological replicates).
f) Western blotting analysis of H3K4me2 in C2C12 cells differentiated to myotubes for 10 DIV in the presence of DHT (10 nM).

H3K4me2 was detected with a specific antibody that recognize the H3 modified K residue. H3 antibody was used as loading control. Graphs show mean±SEM; two-way (b), one-way (c, d, e) ANOVA followed by Tukey HSD tests, or Student t test (f). * p≤0.05; ** p≤0.01; *** p≤0.001.

FIG. 4 shows that LSD1 and PRMT6 synergistically transactivate normal and polyQ-expanded AR.
a) PLA analysis in MN1 cells expressing AR24Q (top graphs) or AR100Q (bottom graphs) and vectors for Lsd1 and Prmt6 silencing. Cells were treated with DHT (10 nM, 16 h). Nuclei were detected with DAPI. Shown are representative images. Bar, 17 μm. Graphs show quantification of nuclei from three independent experiments (AR24Q P6/LSD1 vehicle: n=12; AR24Q P6/LSD1 DHT: n=22; AR24Q Cas9 scrambled AR/LSD1: n=169; AR24Q Cas9 scrambled AR/LSD1: n=198; AR24Q Cas9 AR/PRMT6: n=184; AR24Q Cas9 g2 AR/PRMT6: n=237; AR100Q PRMT6/LSD1 vehicle: n=79; AR100Q PRMT6/LSD1 DHT: n=116; AR100Q Cas9 scrambled AR/LSD1: n=302; AR100Q Cas9 scrambled AR/LSD1: n=190; AR100Q Cas9 AR/PRMT6: n=202; AR100Q Cas9 g2 AR/PRMT6: n=164).
b) Western blots of MN1 cells transduced with lentiviral vectors for Lsd1 and Prmt6 silencing. Shown is one experiment representative of three biological replicates. Quantification is shown at the bottom.
c) Immunoprecipitation of PRMT6 and immunoblotting of the indicated proteins in the skeletal muscle (quadriceps) of 24-week-old WT and AR113Q mice. Shown is one representative experiment (3 mice/genotype).
d) Transcriptional assays in HEK293T cells expressing AR24Q and AR65Q alone (mock) or with LSD1 and PRMT6 and treated for 16 h with vehicle, DHT (10 nM), TCP (10 μM), or Adox (10 μM) (n=3-7 biological replicates).
e) (Left) Transcriptional assay in HEK293T cells expressing AR24Q or AR65Q with or without CRISPR guides to silence Lsd1 and Prmt6 (n=3 biological replicates). (Right) Western blots of PRMT6 levels in HEK293T expressing Cas9 alone or together with guides to silence PRMT6. Shown is one experiment representative of three biological replicates. Quantification is shown at the bottom.

AR, LSD1, and PRMT6 were detected with specific antibodies, and beta-tubulin (□-Tub) and calnexin (CNX) were used as loading controls. Graphs show mean±SEM; Student t test (a, c) or one-way ANOVA followed by Tukey HSD tests (d, e). * p≤0.05; ** p≤0.01; *** p≤0.001.

FIG. 5 shows that silencing of LSD1 and PRMT6 suppresses polyQ-expanded AR neurotoxicity.
a) Analysis of the eye phenotype in flies expressing GFP, AR0Q, or AR52Q with or without RNAi to silence dLsd1 and the PRMT6 fly ortholog Dart8. Shown are representative images from 10-15 flies/genotype.
b) RT-PCR analysis of dLsd1 mRNA transcript levels normalized to Tubulin (n=3 flies/genotype).
c) Disease severity in AR52Q flies with or without dLsd1 and Dart8 silencing (n=10-15 flies/genotype).

Graphs show mean±SEM; Student t-test (b) or one-way ANOVA followed by Tukey HSD test (c). * p≤0.05; *** p≤0.001.

FIG. 6 shows RNAi strategy to silence Lsd1 and Prmt6 in vivo.
a) Western blots of LSD1 and PRMT6 in MN1 cells transfected with vectors expressing scramble amiR or amiR to silence Lsd1 and Prmt6. Shown is one experiment representative of three biological replicates.
b) Cell viability assay in MN1 cells expressing either AR24Q or AR100Q transfected as indicated and treated with DHT (10 nM, 48 h, n=3 biological replicates).
c) Schematic of the AAV9 vector expressing GFP and amiRs to silence both Lsd1 and Prmt6 (amiR-Lsd1 Prmt6). ITR, inverted terminal repeat; WPRE, woodchuck hepatitis virus post-transcriptional regulatory element; pA, poly-adenylation site.
d) Biodistribution of viral particles in different tissues of WT mice.
e) Western blots of GFP expression in the indicated tissues of WT mice transduced by AAV9-amiR. Shown is one experiment representative of three biological replicates in three mice (Q=quadriceps; Sc=spinal cord; Bs=brainstem; L=liver; H=heart; Lg=Lungs; A=Adispose tissue).
f) RT-PCR analysis of transcript levels of Lsd1, Prmt6, mouse AR (mAR), and human AR (hAR) normalized to actin in the quadriceps muscle of 13-week-old AR100Q mice with or without amiR-Lsd1/Prmt6 (n=7-9 mice/group).
g) Western blots of LSD1 and PRMT6 in the skeletal muscle of AR100Q mice treated with or without amiR-Lsd1/Prmt6. Shown is one experiment representative of five mice/group. Quantification is shown at the bottom.

GFP, LSD1, and PRMT6 were detected with specific antibodies, and beta-tubulin (□-Tub) and calnexin (CNX) were used as loading controls. Graphs show mean±SEM; one-way ANOVA followed by Tukey HSD tests (a, b), or Student t test (d, f, g). * p≤0.05; ** p≤0.01; *** p≤0.001.

FIG. 7 shows that silencing of Lsd1 and Prmt6 modifies gene expression in SBMA muscle.
a) Venn diagrams showing intersection of differentially expressed genes (top, upregulated; bottom, downregulated; absolute fold-change >4 and adjusted p≤0.01) obtained by comparing control (AR100Q) versus amiR-treated SBMA mice; AR100Q versus WT mice; or amiR-treated versus WT mice.
b) Ring plot showing the number of differentially expressed genes in AR100Q versus WT mice from differential expression analysis and relative proportions of totally and partially rescued genes.
c) Heatmap of top 20 clusters obtained from functional enrichment analysis of rescued genes. Colour is proportional to enrichment p-values. Each cluster name is based on the most statistically significant term within the cluster.
d) Similarity network of enriched terms. Each node represents an enriched term and is coloured by cluster identifier. Node size is proportional to number of rescued genes in the term. Edge width is proportional with similarity score computed between pairs of nodes.

FIG. 8 shows that silencing of Lsd1 and Prmt6 ameliorates the disease phenotype of SBMA mice.
a) Body weight, hanging wire, and rotarod analysis of WT and AR100Q mice treated with either vehicle or amiR-Lsd1 Prmt6 (n=7-10 mice/group).
b) NADH staining of 8-week-old mice treated as indicated (n=4 mice/group; n=11.000 fibres/group).
c) RT-PCR analysis of denervation markers normalized to Actin in control or AR100Q mice treated with vehicle or amiR-Lsd1/Prmt6 (n=4-9 mice/group).
d) Western blots of AR in the skeletal muscle of AR100Q mice treated with either vehicle or amiR-Lsd1/Prmt6 (n=3 mice/group). HMW, high molecular weight species.

AR was detected with specific antibody and calnexin (CNX) was used as loading controls. Graphs show mean±SEM; one-way ANOVA followed by Tukey HSD tests (a, b, c), or Student t-test (d), * p≤0.05; ** p≤0.01; *** p≤0.001.

FIG. 9 shows that silencing of human LSD1 and PRMT6 modifies gene expression and prostate cancer cell proliferation.
a) Western blots of LSD1 and PRMT6 in HEK293T cells transfected with vectors expressing scramble amiR or amiR-LSD1/PRMT6 to silence LSD1 and PRMT6. Shown is one experiment representative of 3-4 biological replicates. Quantification of the levels of LSD1 (yellow) and PRMT6 (blue) is shown at the bottom.
b) RT-PCR analysis of the indicated genes in HEK293T transfected with vectors expressing scramble amiR or amiR-LSD1/PRMT6 (n=3 biological replicates).
c) BrdU cell proliferation assay performed in LNCaP cells transduced with lentiviral vectors expressing scramble amiR or amiR-LSD1/PRMT6 (n=15-19 fields from three independent experiments).

Graphs show mean±SEM; one-way ANOVA followed by Tukey HSD tests (a, c), or Student t-test (b), * p≤0.05; ** p≤0.01.

FIG. 10 shows a working model of polyQ-expanded AR and co-factor in SBMA muscle. In physiological conditions AR controls the expression of target genes. PolyQ expansions result in the aberrant transcription of AR co-regulators, such as LSD1 and PRMT6, which in turn boost AR transactivation, thus enhancing toxic GOF. Intervention to block this feedforward mechanism ameliorates disease out come in animal models of SBMA.

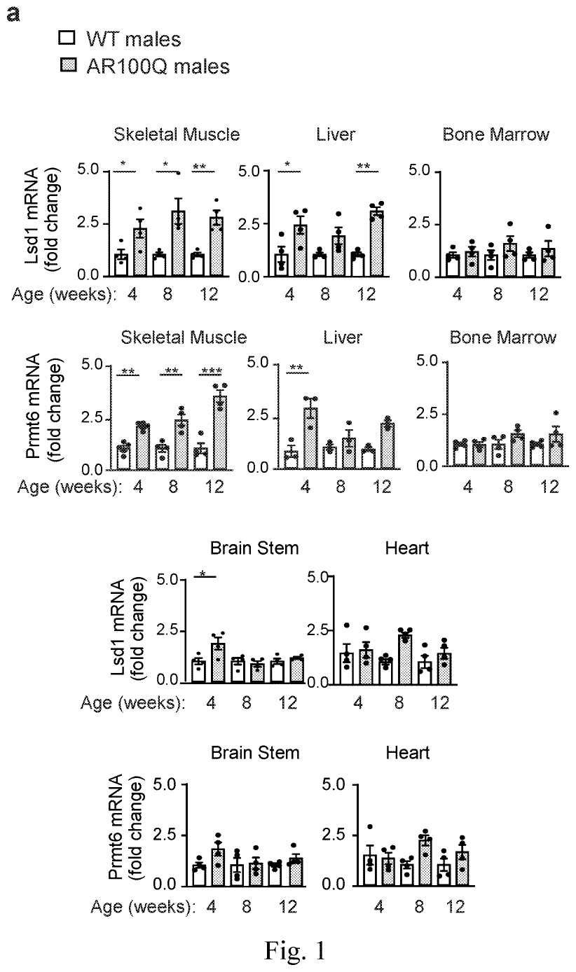

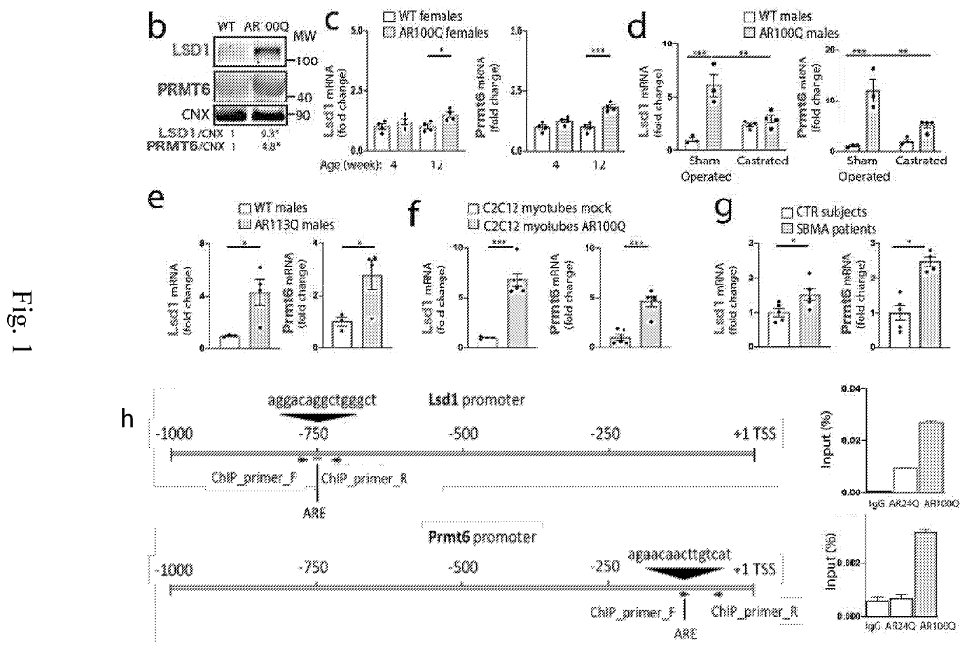

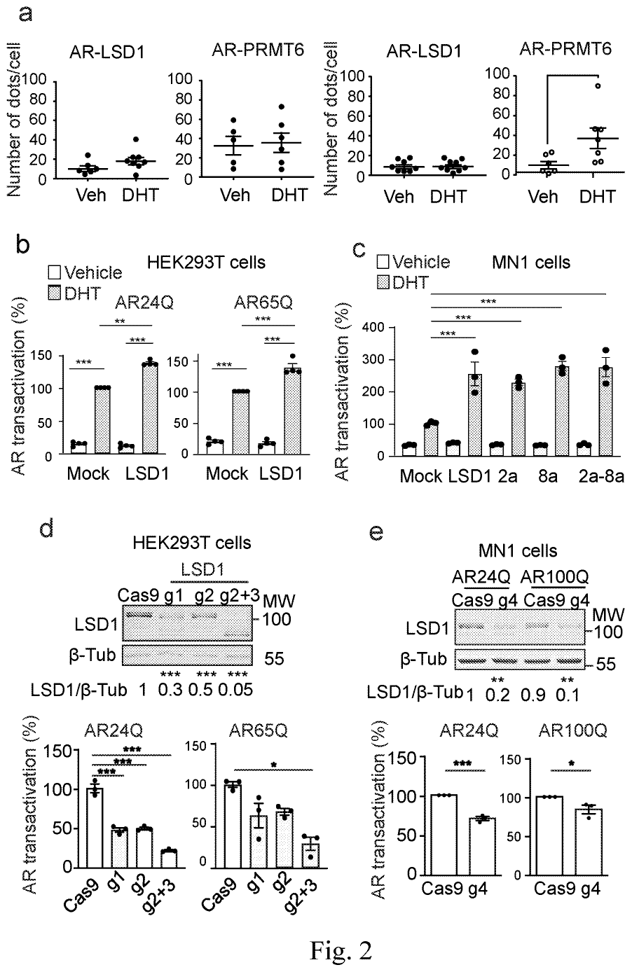

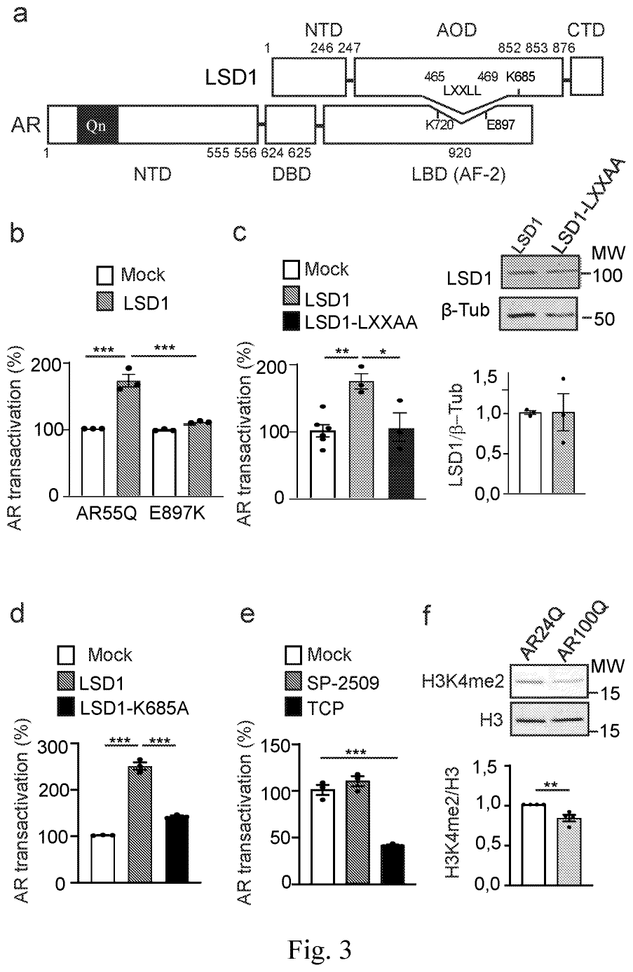

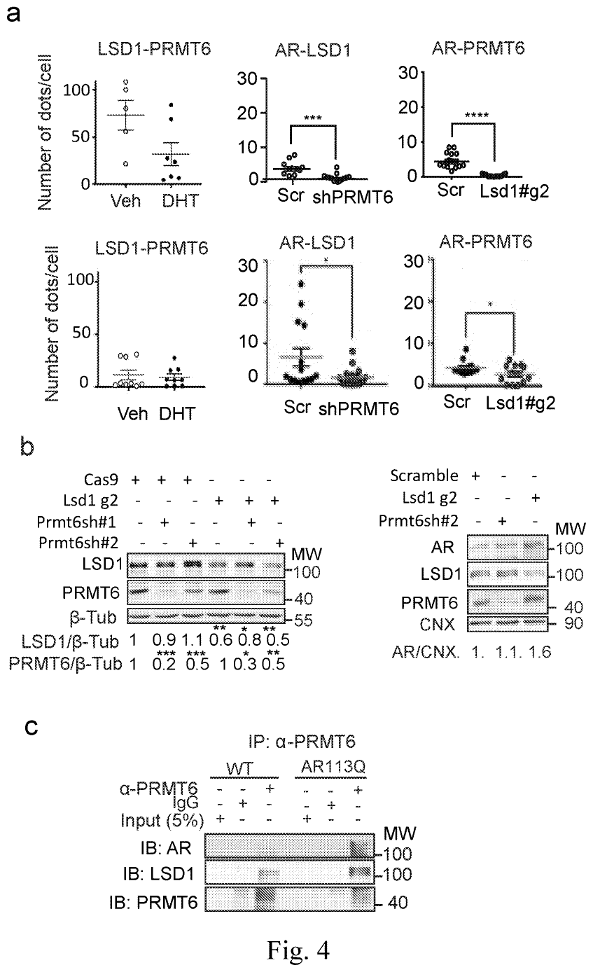

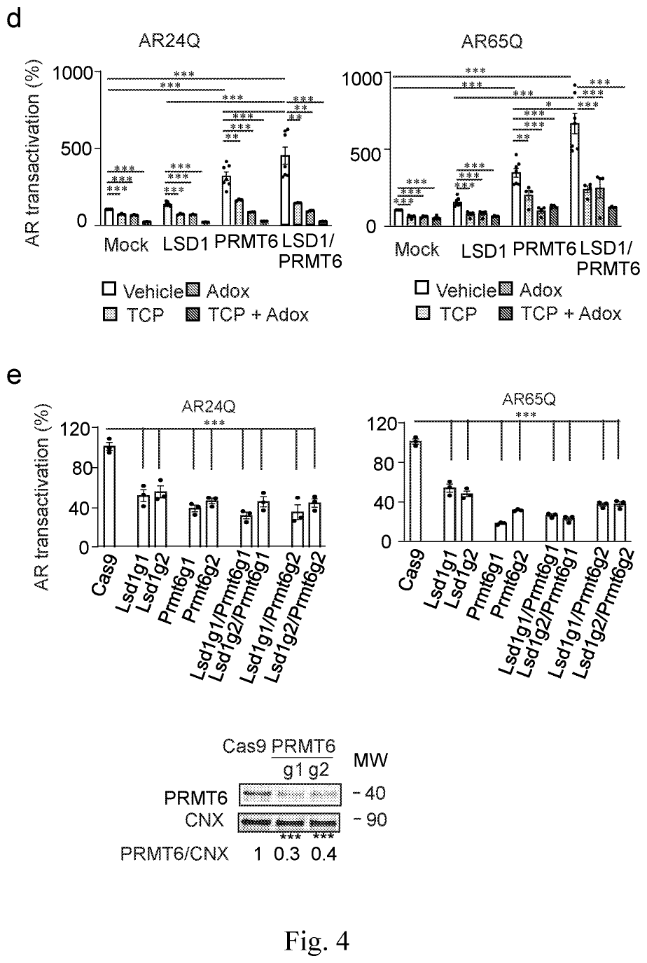

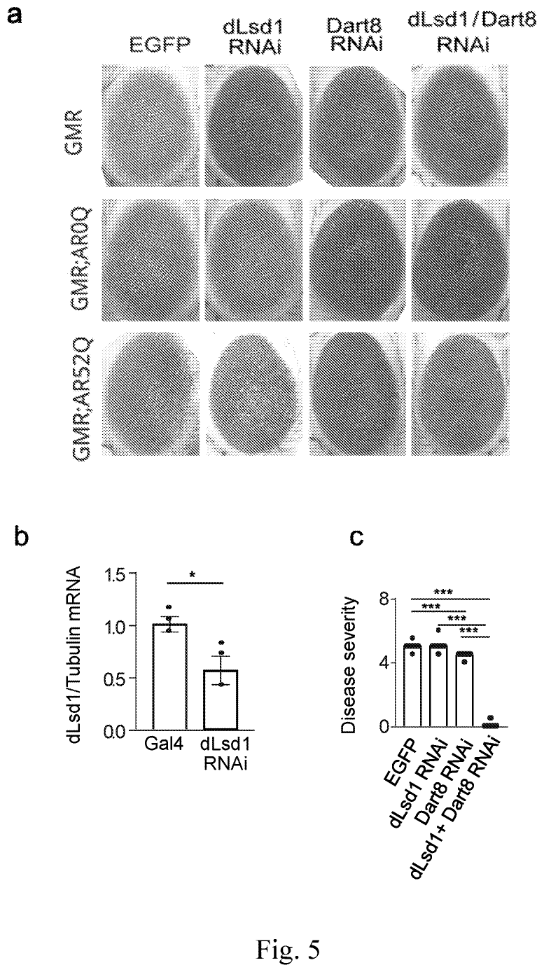

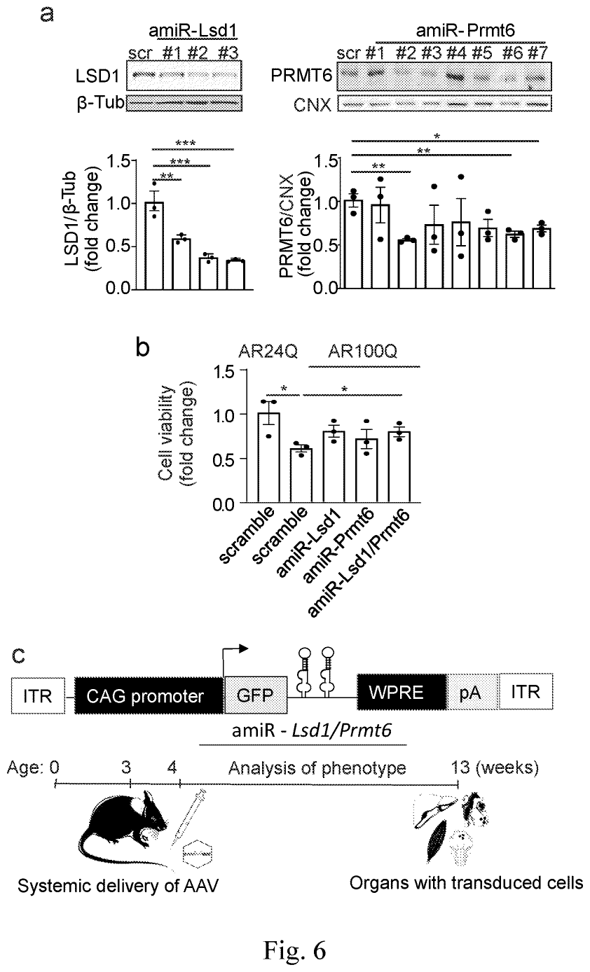

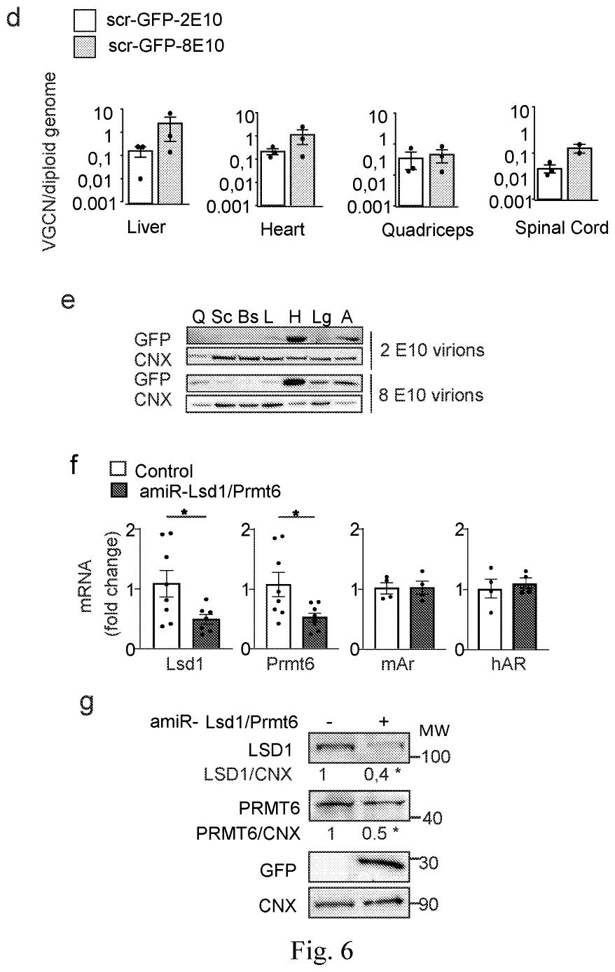

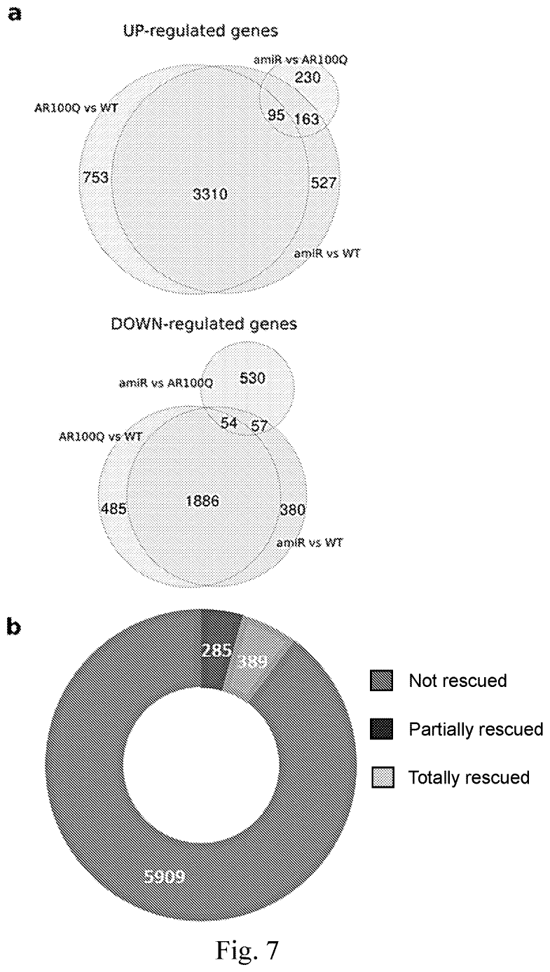

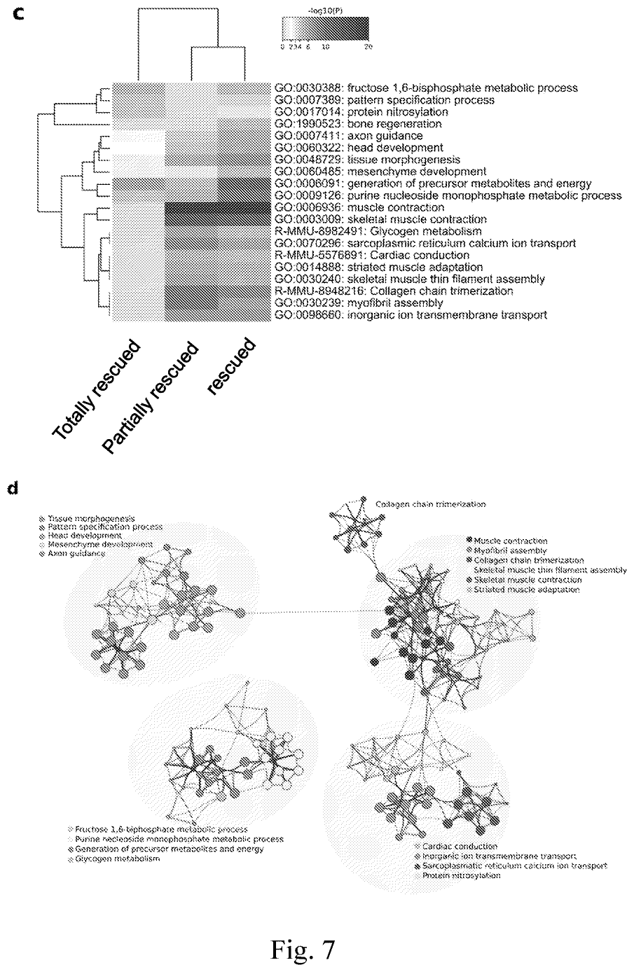

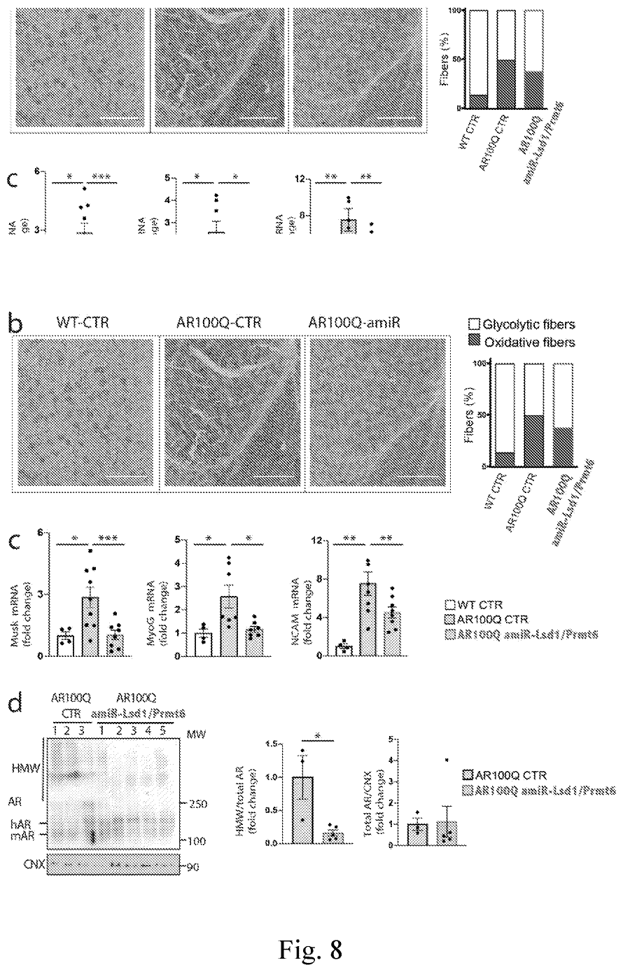

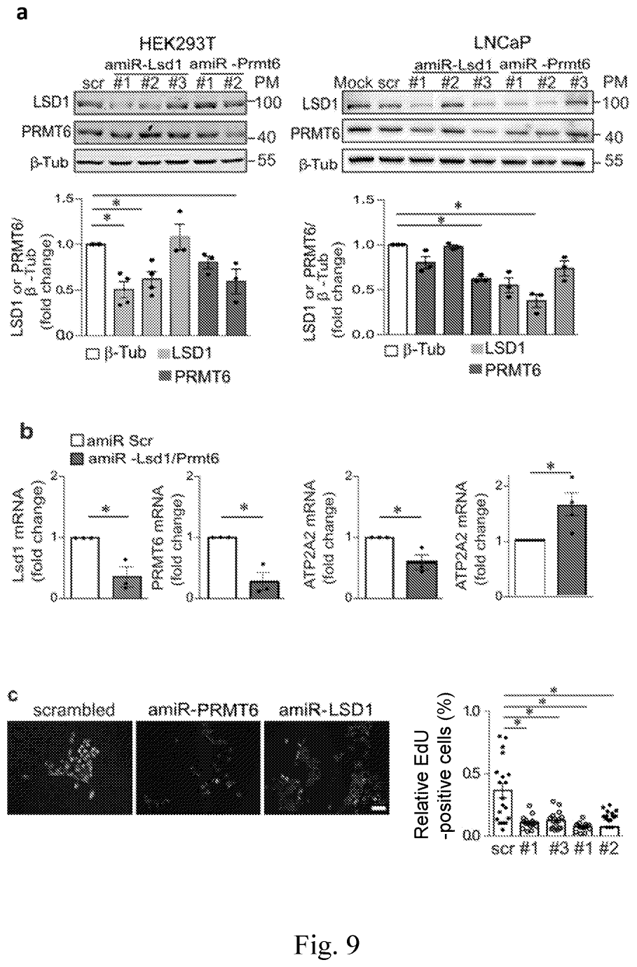

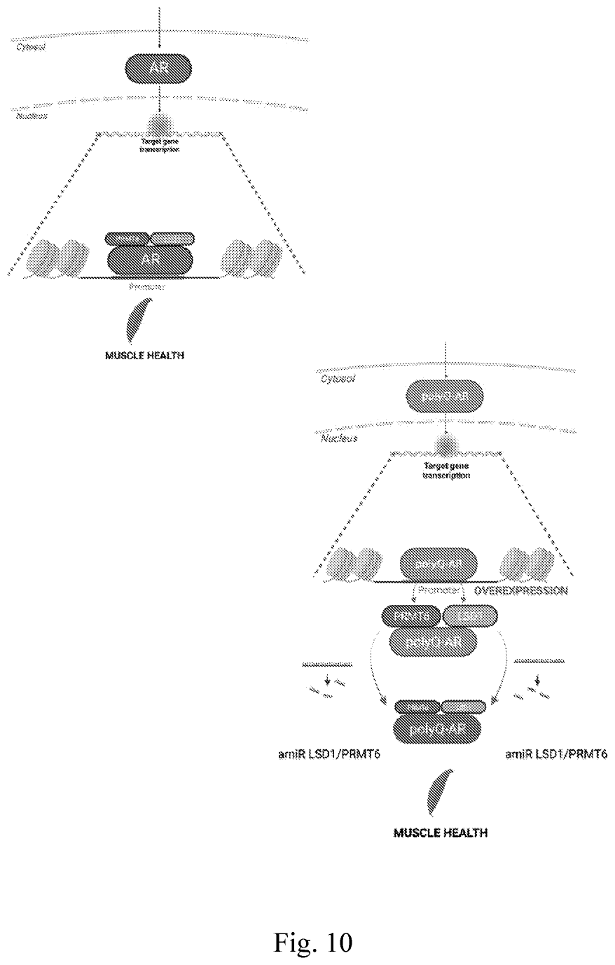

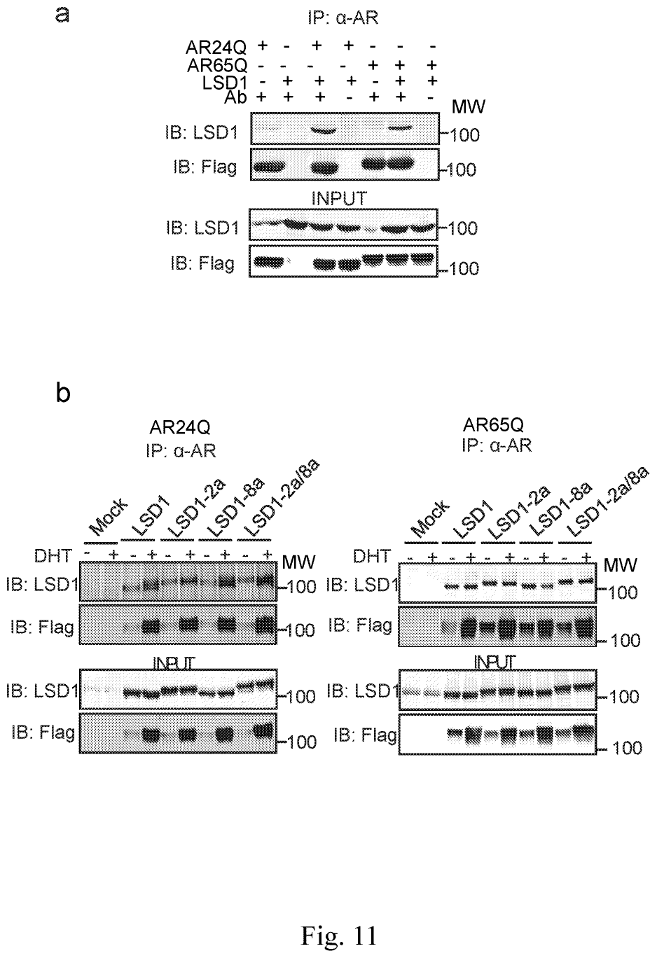

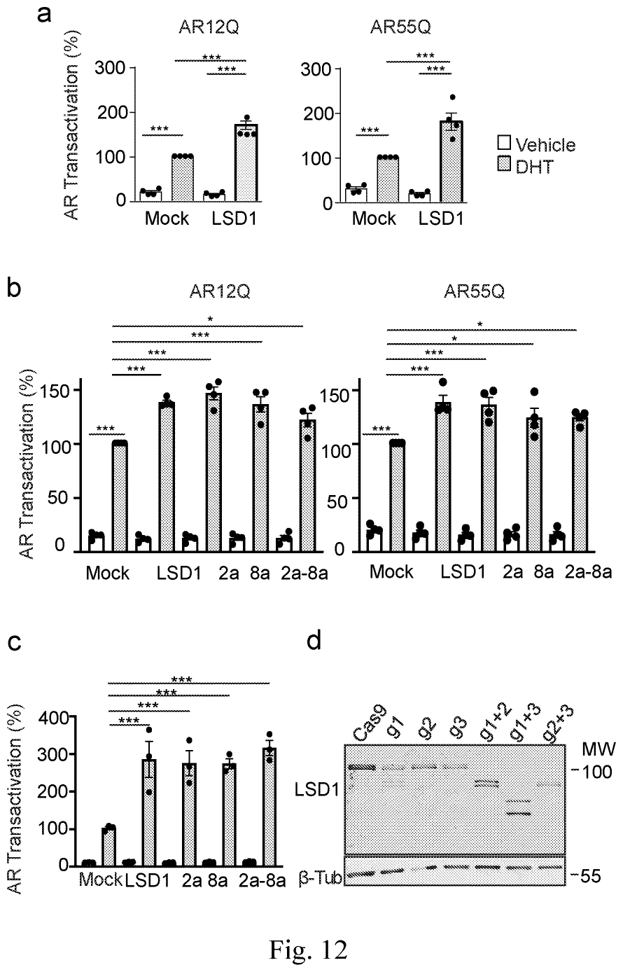

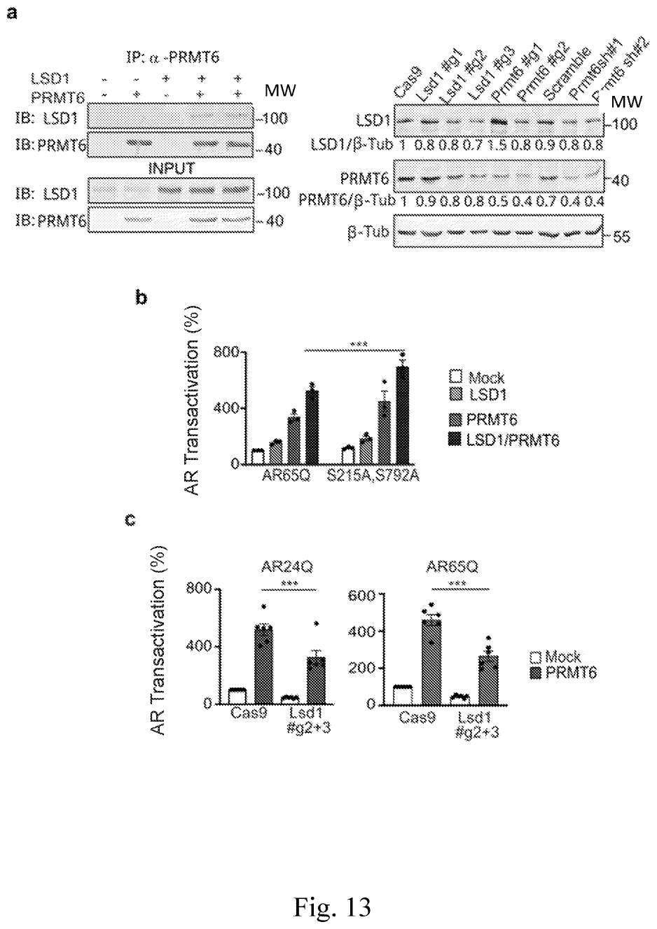

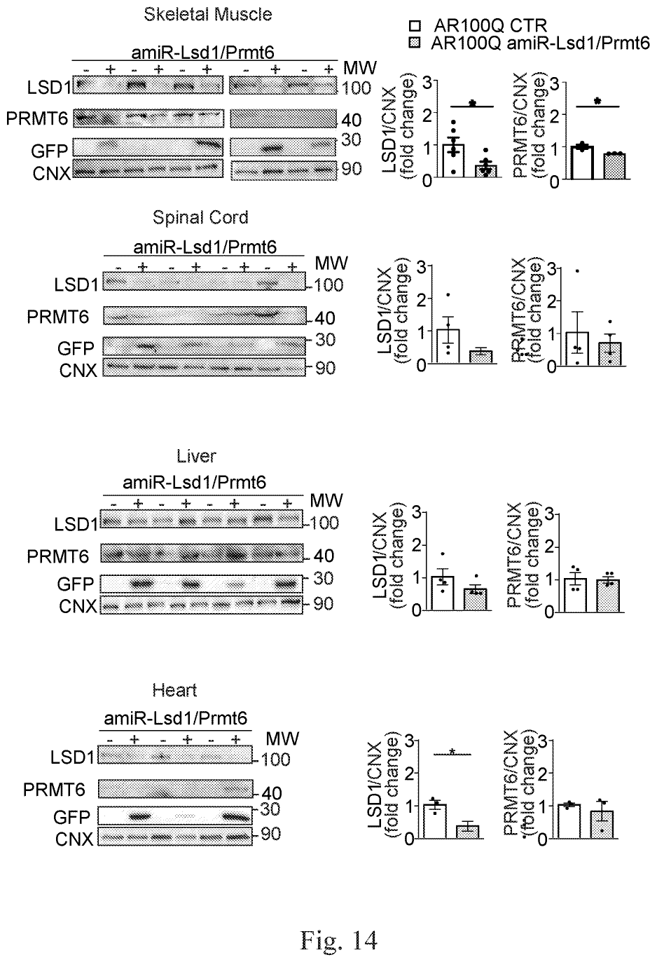

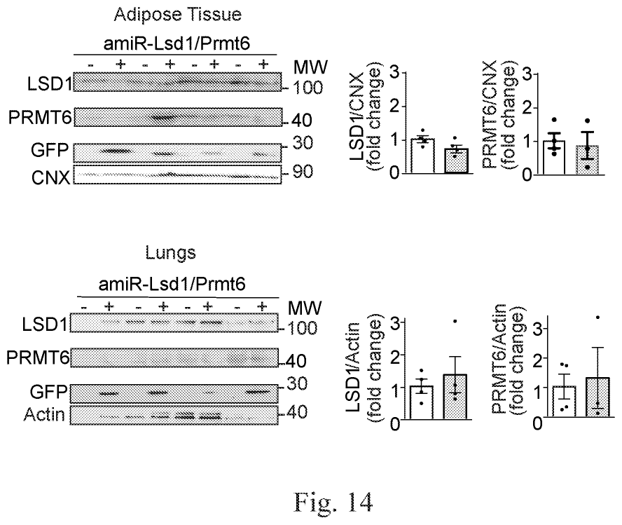

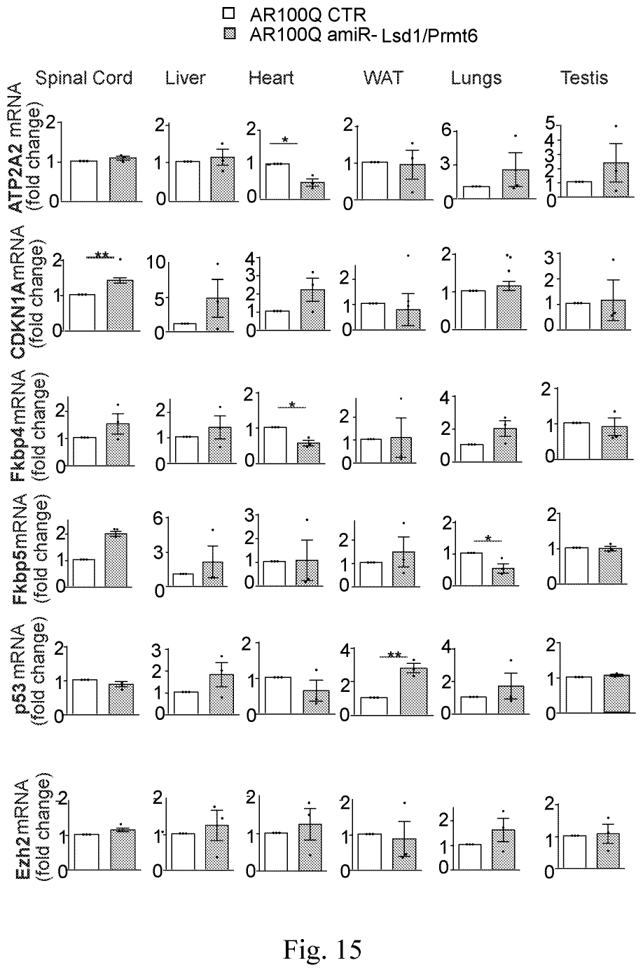

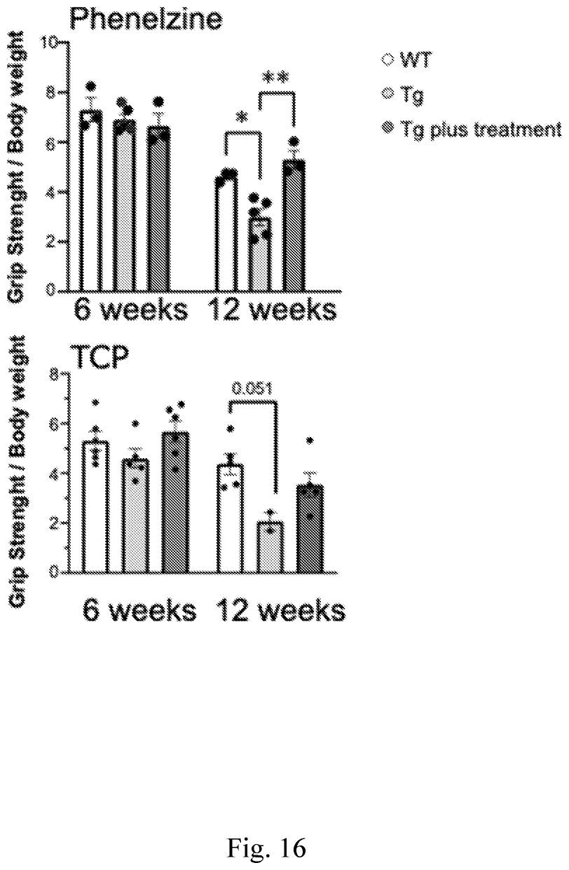

FIG. 11 shows that LSD1 interacts with normal and polyQ-expanded AR.
a-b) Immunoprecipitation (IP) and Western blotting (IB) analysis of Flag-tagged AR and LSD1 interaction in HEK293T cells (n=3 biological replicates).
AR was detected with anti-Flag antibody and LSD1 with a specific antibody.
Molecular weight (MW) is indicated on the right.

FIG. 12 shows that LSD1 is a co-activator of normal and polyQ-expanded AR.
a) Transcriptional assays in HEK293T cells expressing AR12Q and AR55Q driven by the EF1α promoter alone and together with LSD1 and treated with vehicle and DHT (10 nM, 16 h, n=4 biological replicates).
b) Transcriptional assays in HEK293T cells expressing AR24Q and AR65Q alone and together with the indicated LSD1 isoforms and treated with vehicle and DHT (10 nM, 16 h, n=4 biological replicates).
c) Transcriptional assays in MN1 cells expressing AR24Q alone and together with LSD1 and treated with vehicle and DHT (10 nM, 16 h, n=3 biological replicates).
d) Western blotting analysis of LSD1 expression in HEK293T cells stably expressing Cas9 and the indicated guides targeting LSD1. One experiment representative of n=7 biological replicates is shown. LSD1 was detected with a specific antibody, and beta-Tubulin (β-Tub) was used as loading control. Graphs, mean±s.e.m., two-way ANOVA followed by Tukey HSD test, * p≤0.05, *** p≤0.001.

FIG. 13 shows that LSD1 and PRMT6 interact and synergistically transactivate normal and polyQ-expanded AR.
a) (Left) Immunoprecipitation (IP) and immunoblotting (IB) analysis of LSD1 and PRMT6 interaction in HEK293T cells expressing PRMT6 tagged with EGFP and LSD1. (Right) Western blotting analysis of LSD1 and PRMT6 in HEK293T cells transduced with lentiviral vectors to silence LSD1 and PRMT6 by the CRISPR technology. Shown is one experiment representative of 3 (right) and 2 (left) biological replicates. Quantification is shown at the bottom.
b) Transcriptional assay in HEK293T cells expressing AR65Q and AR65Q-S215A, S792A alone (Mock) and with both LSD1 and PRMT6. The cells were treated with DHT (10 nM, 16 h, n=3 biological replicates).
c) Transcriptional assay in HEK293T cells with and without silencing guides targeting LSD1 and expressing AR24Q and AR65Q alone (Mock) and with PRMT6. The cells were treated with DHT (10 nM, 16 h, n=6 biological replicates).

LSD1 and PRMT6 were detected with specific antibodies, and β-Tub was used as loading control. Graphs, mean±s.e.m., two-way ANOVA followed by Tukey HSD test, *** p≤0.001.

FIG. 14 shows the efficacy of amiR target silencing in vivo.

Western blotting analysis in the indicated tissues of AR100Q mice treated with and without amiR-Lsd1/Prmt6, (n=3-5 mice/genotype). LSD1, PRMT6, and GFP were detected with specific antibodies, and CNX or Actin were used as loading controls. Graphs, mean s.e.m., student's t test, * p≤0.05.

FIG. 15 shows the effect of amiR treatment on gene expression.

RT-PCR analysis of LSD1, AR, and PRMT6 target genes in AR100Q mice treated with vehicle or amiR-Lsd1/Prmt6 (n=3 mice/genotype). Graphs, mean±s.e.m., student's t test, * p≤0.05.

FIG. 16 shows the effect of small molecule inhibition of LSD1 on the phenotype of AR100Q mice.

The graphs show the grip strength, normalized to body weight, of wild-type (WT), AR100Q transgenic mice (Tg) and AR100Q transgenic mice treated with phenelzine (a) or Tranylcypromine (TCP) (b). Graphs, mean±s.e.m., student's t test, ** p≤0.01.

### DETAILED DESCRIPTION OF THE INVENTION

The present invention is directed to novel therapeutic agents comprising at least one inhibitor of at least one androgen receptor (AR) coactivator, selected from protein arginine methyltransferase 6 (PRMT6), lysine specific demethylase 1 (LSD1), or both.

Unless defined otherwise, all technical and scientific terms used herein have the same meaning as commonly understood by one of ordinary skill in the art to which this invention belongs.

The term “therapeutic agent” means a substance or combination of substances having properties for treating or preventing disease, especially in human beings.

An “inhibitor”, in accordance with the present invention is an agent capable of directly or indirectly reducing, or suppressing the expression or activity of the inhibited molecule. The term “expression” is used herein in its broadest meaning and comprises the production of RNA or of RNA and protein. With respect to RNA, the term “expression” or “translation” relates in particular to the production of peptides or proteins.

For example, an inhibitor in accordance with the present invention can be a molecule that directly or indirectly reduces or suppresses transcription of a gene (target gene) encoding the molecule to be inhibited (target molecule), or translation of the target molecule from its gene transcript (messenger RNA, mRNA). An inhibitor in accordance with the present invention can also be a chemical entity, such as a small molecule, capable of directly or indirectly reducing or suppressing the biological activity of the target molecule.

As used herein, the term “androgen receptor” preferably refers to human androgen receptor (AR) (UniProt P10275-1).

An androgen receptor coactivator is a protein that interacts with androgen receptors to enhance transactivation of androgen receptor's target genes.

The therapeutic agents of the invention are inhibitors of PRMT6 and/or LSD1 AR coactivators.

As used herein, the term “PRMT6” preferably refers to human protein arginine methyltransferase 6 protein (UniProt Q96LA8), or to the encoding gene (NCBI Reference Sequence: NC_000001.11) or transcript (NCBI Reference Sequence: NM_018137.3, SEQ ID NO:1) thereof.

As used herein, the term “LSD1” preferably refers to the human lysine specific demethylase 1 protein (UniProt 060341), also known as “KDM1A”, to the encoding gene (NCBI Reference Sequence: NG_047129.1) or transcript (NCBI Reference Sequence: NM_001009999.3, SEQ ID NO:2) thereof, or to any variant or isoform thereof, such as LSD1-2a, LSD1-8a, and LSD1-2a/8a isoforms deriving from alternative splicing¹⁰.

The therapeutic agent of the invention is preferably for use in the treatment of a disease associated with gain-of-function of AR and/or with overexpression of an AR coactivator in a subject in need thereof, said AR coactivator being preferably PRMT6 or LSD1.

As used herein, “disease”, “disorder” and “condition” are used interchangeably, to indicate an abnormal state in a subject.

Diseases associated with “gain-of-function” of AR are diseases characterized by an activity of AR that is increased compared to physiological baseline. Such diseases can be caused by mutant AR, wherein mutation increases the functionality of AR. An example of such diseases is a polyglutamine (polyQ) disorder, which is associated with “gain-of-function” of mutant gene or product thereof, such as SBMA for AR. Further disease associated with AR gain of function include hormone-dependent cancers, such as prostate, bladder, liver or kidney cancers, metabolic syndrome, diabetes, and insulin resistance.

Preferably, the therapeutic agents of the invention are for use in the treatment of a disease associated with AR gain of function, more preferably SBMA or a hormone-dependent cancer, such as prostate, bladder, liver or kidney cancers, or a metabolic syndrome, diabetes, or insulin resistance.

The term “overexpression” means increased expression of a gene beyond the norm. Assays for determining overexpression of a gene are well known in the art and include, but are not limited to, immunological assays, nuclease protection assays, northern blots, in situ hybridization, and Real-Time Polymerase Chain Reaction (RT-PCR), expressed sequence tag (EST) sequencing, cDNA microarray hybridization or gene chip analysis, subtractive cloning, Serial Analysis of Gene Expression (SAGE), Massively Parallel Signature Sequencing (MPSS), and Sequencing-By-Synthesis (SBS). A differentially expressed gene may be overexpressed as compared to the expression level of a normal or control cell or to an internal control. For instance, the term refers to a differential that is about 1.5 times, about 2.0 times, about 3.0 times, about 5 times, about 10 times, about 50 times, or yet more than about 100 times higher than the expression level detected in a control sample. A “control” is used in an experiment for comparison or normalization purposes. Controls for use in comparing gene expression at the mRNA level include internal and external controls. An internal control refers to a gene known to be present in the sample to be tested. The expression level of the gene is preferably well characterized and provides a reliable measure of gene expression level in the control. Examples of genes that are useful as internal controls include, but are not limited to, housekeeping genes such as β-actin, 18S, glyceraldehyde-3-phosphate dehydrogenase (GAPDH), and cyclophilin. External controls include use of a subject or a sample from a subject, known to express the gene of interest at certain level, or for instance a sample from a healthy subject.

Diseases associated with overexpression of an AR coactivator include SBMA, prostate cancer, lung cancer, breast cancer, bladder cancer, cervical cancer, and lymphoma.

Preferably, the therapeutic agents of the invention are for use in the treatment of a disease associated with overexpression of an AR coactivator, more preferably SBMA, prostate cancer, lung cancer, breast cancer, bladder cancer, cervical cancer, or lymphoma.

More preferably, the therapeutic agents of the invention are for use in the treatment of a disease associated with either AR gain of function and overexpression of AR coactivators, such as SBMA or cancer, most preferably SBMA or prostate cancer.

As used herein, “treatment” (and grammatical variations thereof such as “treat” or “treating”) refers to the administration of a therapeutic agent or formulation according to the invention to obtain a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease or control of disease progression. The term “treatment” then includes “prevention”, i.e., inhibition or delay of the inception or decrease of the occurrence of a disease in a subject. Prevention may be complete (e.g., the total absence of pathological cells in a subject) or partial. Prevention also refers to a reduced susceptibility to a clinical condition. Control of disease progression is understood as the achievement of the beneficial or desired clinical results that include, but are not limited to, reduction of the symptoms, reduction of the duration of the disease, stabilization of pathological states (specifically to avoid additional deterioration), delay of the progression of the disease, improvement in the pathological state, and remission (both partial and total). The control of progression of the disease also involves an extension of survival, compared with the expected survival if treatment is not applied.

In particular, in accordance with the present invention, the terms “treatment”, “treating”, “treat” and the like, as used herein, preferably refer to the administration of a therapeutic agent or formulation of the invention to cure, prevent, delay and/or control the clinical manifestations of the disease to be treated. For example, in the case of cancer, treatment includes a reduction in cachexia, increase in survival time, elongation in time to tumor progression, reduction in tumor mass, reduction in tumor burden and/or a prolongation in time to tumor metastasis, each as measured by standards set by National Health Institutes, e.g., by the National Cancer Institute and the U.S. Food and Drug Administration for the approval of new drugs. In the case of SBMA, treatment includes a reduction or prevention of SBMA symptoms, including early symptoms such as one or more of weakness/cramps in arm and leg muscles, face, mouth, and tongue muscle weakness, difficulty with speaking and swallowing, twitching (Fasciculations), tremors and trembling in certain positions, enlarged breasts, (gynecomastia), numbness, infertility, and testicular atrophy.

The term “effective amount” refers to an amount of a substance sufficient to achieve the intended purpose. The effective amount of a given substance will vary with factors such as the nature of the substance, the route of administration, the size and species of the animal to receive the substance and the purpose of giving the substance. The effective amount in each individual case may be determined empirically by a skilled artisan according to established methods in the art. For example, by “therapeutically effective dose or amount” of a compound, composition or formulation according to the invention, is intended an amount that, when administered as described herein, brings about a positive therapeutic response, such as improved recovery from the disease or from side conditions of the disease.

Those in need of treatment include those already inflicted as well as those in which prevention is desired (e.g., those with no symptoms but diagnosed with the genetic disorder, etc.).

“Patient” or “subject” as used herein means a male or female human, non-human animal, and animal models used for clinical research. In one embodiment, the subject of treatment is a human diagnosed with a disease associated with AR gain of function and/or AR-coactivators overexpression. In certain embodiments, the human subject is a prenatal, a newborn, an infant, a toddler, a preschool, a grade-schooler, a teen, a young adult or an adult.

Preferably, the subject is a male.

According to preferred embodiments, the at least one inhibitor of at least AR coactivator, for use in accordance with the present invention, is a genetic silencer. A “genetic silencer” is an agent capable of specifically targeting a gene or transcript and of inhibiting, reducing or disrupting expression of the targeted gene or transcript. Preferably, a genetic silencer according to the invention reduces the amount or the activity of its target by no more than 90%, no more than 80%, no more than 70%, no more than 60%, or no more than 50%, compared to a non-inhibited target; more preferably, a genetic silencer according to the invention reduces the amount or the activity of its target by 10-70%, by 10-60%, by 10-50%, compared to a non-inhibited target.

A genetic silencer, according to the invention, can be an RNA interfering molecule, an antisense oligonucleotide, or a genome editing agent, capable of targeting a transcript or a gene, respectively, inhibiting the same.

In accordance with the present invention, a gene or transcript is “targeted” by a genetic silencer and a genetic silencer is “targeting” a gene or transcript, when the genetic silencer is capable of selectively decreasing or inhibiting the expression of the target gene or of an allele of the target gene or the translation of the target transcript, and/or when the genetic silencer hybridizes under stringent conditions to the target gene or transcript. For example, a genetic silencer targeting an AR coactivator (such as PRMT6 and/or LSD1), in accordance with the present invention, selectively decreases or inhibits the expression of a gene, or of an allele of a gene, encoding the AR coactivator, or selectively decreases or inhibits the translation of a transcript of the AR coactivator; alternatively or in addition, a genetic silencer targeting an AR coactivator (such as PRMT6 and/or LSD1), in accordance with the present invention, hybridizes under stringent conditions to the AR coactivator gene or transcript. Stringent conditions typically mean prehybridization and hybridization at 42° C. in 5×SSPE, 0.3% SDS, 200 pg/ml sheared and denatured salmon sperm DNA, and either 25% 35% or 50% formamide for low to medium to high stringencies respectively. Subsequently, the hybridization reaction is washed three times for 30 minutes each using 2×SSC, 0.2% SDS and either 55° C., 65° C., or 75° C. for low to medium to high stringencies.

As used herein, a “target sequence” is a nucleotide sequence that is targeted by a genetic silencer according to the invention: herein a target sequence can be indicated as the sequence of cDNA (positive strand, 5′ to 3′) corresponding to the RNA target transcript (sense transcript, 5′ to 3′) of a target gene.

As used herein, a “targeting sequence” is a sequence of a genetic silencer that is (completely or partially) complementary to the target sequence, or transcript thereof, and that is capable of directing the genetic silencer to the target gene or transcript.

The terms “sequence” “nucleotide sequence” or “isolated nucleotide sequence” or “polynucleotide sequence” or “polynucleotide” or “isolated polynucleotide sequence” are interchangeably used herein and refer to a nucleic acid molecule, either DNA or RNA, containing deoxyribonucleotides or ribonucleotides respectively. The term “sequence” may be used herein to indicate a polynucleotide or portion of a polynucleotide having a certain sequence, for brevity.

A nucleic acid may be double stranded, single stranded, or contain portions of both double stranded or single stranded sequence.

Unless differently indicated, sequences of double stranded nucleic acids herein indicated are the 5′ to 3′ sequences of the sense (or positive) strand.

A genetic silencer can be tested either in vitro or in vivo for the ability to target a gene or transcript, by techniques known in the art.

Preferably, the therapeutic agent comprises at least one genetic silencer being an RNA interfering (RNAi) molecule.

RNA interference is a well-known and natural process in the cells. In nature, i.e., in plants, animals and some viruses, RNA silencing and post-transcriptional regulation of gene expression is affected by miRNAs, that is a small single-stranded non-coding RNA molecule (containing about 22 nucleotides). miRNAs function via base-pairing with complementary sequences within mRNA molecules. As a result, these mRNA molecules are silenced, by one or more of the following processes: (1) cleavage of the mRNA strand into two pieces, (2) destabilization of the mRNA through shortening of its poly(A) tail, and (3) less efficient translation of the mRNA into proteins by ribosomes. miRNAs are each processed from a longer precursor RNA molecule (“precursor miRNA” or pre-miRNA”): endogenous miRNA genes are transcribed by RNA polymerase II to yield primary miRNAs (pri-miRNAs) that go through an initial nuclear maturation stage, resulting in imperfectly base-paired stem-loop precursors (pre-miRNAs) of ~70 nt, having in fact two regions of complementarity that enables them to form a stem-loop-like structure. After export into the cytoplasm, pre-miRNAs undergo a further maturation step, executed by enzymes called Dicer and Drosha in animals, that cleave the precursor's loop and generate short imperfect double-stranded RNAs (dsRNAs) also called miRNA duplex. In the last maturation step, one of the duplex RNA strands is incorporated into the RISC complex, responsible for translational repression and RNA degradation. The processed miRNA (mature miRNA) pairs to a target mRNA leading to its silencing.

According to the present invention, the term “RNAi molecule” includes synthetic small interfering RNAs (siRNAs), short hairpin RNAs (shRNAs), or artificial miRNAs (amiRs), capable to inhibit the target transcript. The RNAi molecule comprises or consists of a short sequence (targeting sequence) that is complementary to a target sequence within the RNA transcript of the target gene. The targeting sequence of the RNAi molecule can be a sequence that is perfectly complementary to the target sequence, or a degenerate RNAi sequence that targets homologous regions in the target transcript. For example, mismatches and/or wobble base pairs can provide additional targeting sequences. Preferably, the RNAi molecule of the invention comprises or consists of a targeting sequence that is perfectly complementary to the target sequence, or transcript thereof.

The assessment of the inhibition of the target transcript by the RNAi molecule, can be performed using classical molecular biology techniques such as (real time Polymerase Chain Reaction) qPCR, microarrays, bead arrays, or Northern blot analysis or cloning and sequencing, quantifying the RNAi molecule or the target transcript or a substrate of, or compound known to be associated with, the target transcript, in a cell.

The genetic silencer according to the invention can be a RNAi molecule as such (a “mature” RNAi molecule), typically a single-stranded or double-stranded RNA molecule. Moreover, the genetic silencer can be a “precursor” of the mature RNAi molecule: the precursor has two regions of self-complementarity that enables them to form a stem-loop-like structure, which is cleaved by enzymes called Dicer and Drosha in animals; the processed RNAi (mature RNAi molecule), that is the active molecule comprising or consisting of the targeting sequence, is typically a portion of the stem.

Furthermore, the genetic silencer can be a “source” of an RNAi molecule or of a precursor thereof, that may be in the form of a DNA sequence comprising the sequence encoding the RNAi molecule, preferably extending at least 1 to 5 nucleotides of coding sequence upstream and/or downstream of the predicted sequence encoding the RNAi molecule. In some embodiments, RNAi source molecules have up to 1, 2, 3, 4, 5, 6, 7, or more contiguous nucleotides, or any range derivable therein, that flank the sequence encoding the predominant processed mature RNAi or precursor thereof, on one or both sides (5′ and/or 3′ end).

According to preferred embodiments of the invention, the therapeutic agent comprises at least one genetic silencer being an RNAi molecule, or a precursor or source thereof, or an equivalent thereof, targeting an AR coactivator, more preferably PRMT6. Preferably, said RNAi molecule is targeting any one of sequences SEQ ID NO: 3-53, more preferably any one of sequences SEQ ID NO: 18, 21 or 29, or a transcript thereof. Preferably, said RNAi molecule comprises or consists of a polynucleotide having sequence perfectly complementary to any one of sequences SEQ ID NO:3-53, more preferably to any one of sequences SEQ ID NO: 18, 21 or 29, or to the transcript thereof. Most preferably, said RNAi molecule comprises or consists of sequence SEQ ID NO: 171-173, or equivalent thereof. Optionally, the therapeutic agent comprising at least one genetic silencer being an RNAi molecule, or a precursor or source thereof, or an equivalent thereof, targeting PRMT6 further comprises at least one inhibitor of LSD1, preferably said inhibitor of LSD1 being a genetic silencer or a small molecule, more preferably being a genetic silencer.

According to preferred embodiments of the invention, the therapeutic agent comprises at least one genetic silencer being an RNAi molecule, or a precursor or source thereof, or an equivalent thereof, targeting LSD1. Preferably, said RNAi molecule is targeting any one of sequences SEQ ID NO: 54-138, more preferably any one of sequences SEQ ID NO: 68, 72 or 90, or a transcript thereof. Preferably, said RNAi molecule comprises or consists of a polynucleotide having sequence perfectly complementary to any one of sequences SEQ ID NO: 54-138, more preferably to any one of sequences SEQ ID NO: 68, 72 or 90, or transcripts thereof. Most preferably, said RNAi molecule comprises or consists of sequence SEQ ID NO: 174-176, or equivalent thereof. Optionally, the therapeutic agent comprising at least one genetic silencer being an RNAi molecule, or a precursor or source thereof, or an equivalent thereof, targeting LSD1 further comprises at least one inhibitor of PRMT6, preferably said inhibitor being a genetic silencer or a small molecule, more preferably being a genetic silencer.

Equivalents of the RNAi molecule, precursor or source thereof, are also included in the invention.

The term “equivalent” when referred to RNAi molecules is meant to indicate a chemically modified RNAi molecule or a RNAi nucleotide analogue, maintaining the same activity of the RNAi molecule, or a RNAi molecule comprising a degenerated targeting sequences or a sequence with one or more additions, substitutions (generally conservative in nature) and/or deletions, or a sequence that has a high degree of sequence homology to the reference sequence, e.g., sequence homology of at least 80%, at least 85%, at least 90% homology to the targeting sequence of the RNAi molecule. The terms “% sequence identity”, “% identity” or “% sequence homology” refer to the percentage of nucleotides or amino acids of a candidate sequence that are identical to the nucleotides or amino acids in the sequence of reference, after aligning the sequences to achieve the maximum % sequence identity. In a preferred embodiment, sequence identity is calculated based on the full length of two given sequences or on part thereof.

The % sequence identity can be determined by any methods or algorithms established in the art, such as the ALIGN, BLAST and BLAST 2.0 algorithms. Herein, the “% sequence identity”, “% identity” “or “% sequence homology” is calculated dividing the number of nucleotides or amino acids that are identical after aligning the sequence of reference and the candidate sequence, by the total number of nucleotides or amino acids in the sequence of reference and multiplying the result by 100. Preferably the equivalent's sequence has greater than 90%, 95%, 99% sequence identity with the RNAi molecule's sequence.

Several chemical modifications that are well known in the art, aimed at increasing stability or availability of the dsRNA oligonucleotides, may be made.

Also, RNAi molecules of the invention specifically contemplate the use of nucleotides that are modified to enhance their activities. Such nucleotides include those that are at the 5′ or 3′ terminus of the RNAi molecule as well as those that are internal within the molecule.

Modified nucleotides used in the complementary strands of a double strand RNAi molecule either block the 5′OH or phosphate of the RNA or introduce internal sugar modifications that enhance uptake of the active strand of the RNAi molecule. Modifications for the RNAi molecules include internal sugar modifications that enhance hybridization as well as stabilize the molecules in cells and terminal modifications that further stabilize the nucleic acids in cells.

Equivalents of RNAi molecules according to the invention thus include RNAi molecules comprising modified nucleotides.

Equivalents of RNAi molecules according to the invention also include RNAi molecules comprising modified nucleotides termed UNA (unlocked nucleic acid): UNA are acyclic analogues of RNA in which the bond between the C2′ and C3′ atoms has been cleaved, decreasing binding affinity towards a complementary strand, as described in WO2008/147824. UNA are compatible with RNase H recognition and RNA cleavage and improves siRNA mediated gene silencing.

Equivalents also include RNAi molecules according to the invention comprising morpholino nucleic acid analogues, which contain both uncharged and cationic inter-subunit linkages, as described in WO2008/036127, Zip Nucleic Acids (ZNA), containing conjugating spermine derivatives as cationic moieties (Z units) to an oligonucleotide (WO/2007/069092 and EP2075342). Additional teachings for RNAi equivalents are provided in U.S. Pat. No. 5,728,525, which describes nucleoside analogs that are end-labelled, U.S. Pat. Nos. 5,637,683, 6,251,666, which describe L-nucleotide substitutions, and U.S. Pat. No. 5,480,980, which describes 7-deaza-2′-deoxyguanosine nucleotides and nucleic acid analogs thereof. The use of other nucleotide analogs is specifically contemplated for use in the context of the present invention. They include, but are not limited to: ribose modifications (such as 2′F, 2′ H2, 2′N3,4′thio, or 2′ 0-CH3) and phosphate modifications (such as those found in phosphorothioates, methyl phosphonates, and phosphoroborates. Such analogs confer stability on RNAs by reducing or eliminating their capacity to be cleaved by ribonucleases. When these nucleotide analogs are present in RNAi molecules, they can have profoundly positive effects on the stability of the RNAi molecules in animals.

Equivalents also include equivalents of the RNAi molecule precursors or sources thereof, such as codon-optimized sequences and sequences comprising mutated or added nucleotides, e.g., for cloning needs.

The RNAi molecules according to the invention can be obtained from commercial RNA oligo synthesis suppliers. Alternatively, the RNAi molecules according to the invention can be expressed in cells by transfecting the cells with vectors containing a RNAi source, such as a transgene, for expressing the RNAi precursor under the control of a suitable promoter.

The term “promoter” must be understood as a nucleic acid fragment that functions to control the transcription of one or more polynucleotides e.g. coding sequences, which is placed 5′ upstream of the polynucleotide sequence(s), and which is structurally identified by the presence of a binding site for DNA dependent RNA polymerase, transcription initiation sites and, but not limited to, binding sites for transcription factors, repressors, and any other nucleotide sequences known in the art to act directly or indirectly to regulate the amount of transcription from the promoter. A promoter is said to operatively linked to a nucleotide sequence or to drive the expression of it when it can initiate transcription of said nucleotide sequence in an expression system using a gene construct comprising said promoter operably linked to a nucleotide sequence of interest using a suitable assay such a RT-qPCR or Northern blotting (detection of the transcript).

Preferably, the therapeutic agent comprises at least one RNAi molecule precursor in the form of a stem-loop polynucleotide, consisting of e.g., 50 to 80 nucleotides in length, or 50 to 70 nucleotides, or 50 to 65 nucleotides in length, that comprise the targeting sequence.

Preferably, the RNAi precursor polynucleotide comprises (5′ to 3′) about 5 nucleotides flanking a targeting sequence, the targeting sequence, preferably of about 21 nucleotides, that corresponds to the mature RNAi molecule sequence, a loop sequence of 19-22 nucleotides, and the sense target sequence of 19-21 nucleotides optionally including a mismatch in respect of the targeting sequence; preferably the sense target sequence is the reverse complement of the targeting sequence with one, two, or three nucleotides being mismatched; for instance a sense target sequence in a RNAi precursor molecule can comprise nucleotides 1-8 of the reverse complement of a 21 nucleotides-long targeting sequence followed by nucleotides 11-21 of the reverse complement of said 21 nucleotides-long targeting sequence.

Preferably, the at least one genetic silencer is a source of a RNAi molecule, said source being a DNA molecule encoding the RNAi molecule or precursor thereof. The DNA molecule encoding the RNAi molecule or precursor thereof is preferably comprised in a vector. The term “vector” is used to refer to a carrier nucleic acid molecule into which a nucleic acid sequence can be inserted for introduction into a cell where it can be replicated. Vectors include plasmids, cosmids, viruses (bacteriophage, animal viruses, lentivirus, and plant viruses), and artificial chromosomes (e.g., YACs). One of skill in the art would be well equipped to construct a vector through standard recombinant techniques, which are described in Sambrook, 2003, Sambrook, 2001 and Sambrook, 1989, which are hereby incorporated by reference. According to preferred embodiments, the DNA molecule encoding the RNAi molecule or precursor thereof is comprised in a viral vector for delivery in a cell and expression of the precursor in the cell.

According to preferred aspects of the invention, the RNAi molecule source comprises or consists of an expression cassette, or of a vector genome comprising an expression cassette, said expression cassette comprising a nucleic acid sequence encoding the RNAi molecule or precursor thereof, operably linked to regulatory sequences which direct expression of the nucleic acid sequence in the subject.

In some embodiments, the RNAi molecule source comprises a nucleic acid containing more than one sequence encoding for a RNAi molecule or precursor thereof, for example the RNAi molecule source comprises a nucleic acid molecule containing a first sequence encoding a RNAi molecule or precursor thereof and a second sequence encoding a first RNAi molecule or precursor thereof, preferably wherein the first RNAi molecule or precursor thereof comprises a PRMT6 targeting sequence and the second RNAi molecule or precursor thereof comprises a LSD1 targeting sequence.

Preferably the RNAi molecule source comprises or consists of at least one vector, more preferably at least one viral vector, comprising the at least one nucleic acid encoding for a precursor of a RNAi molecule targeting PRMT6 and/or LSD1.

Preferably, the genetic silencer of the invention is an artificial miRNA (amiR), or a precursor or source thereof, or an equivalent thereof amiRs comprise a target-specific siRNA insert (whose sequence comprises the targeting sequence) and a scaffold, based on a natural primary miRNA (pri-miRNA). The target-specific siRNA insert serve as a guide to search for complementary sequences in transcripts, whereas pri-miRNA scaffolds ensure proper processing and transport. The dynamics of siRNA maturation and siRNA levels in the cell resemble those of endogenous miRNAs; therefore, amiRs are safer than other RNAi molecules. Delivered e.g., by viral vectors and expressed under polymerase II (Pol II) promoters, amiRs provide long-lasting silencing. Preferably expression in selected tissues is achieved by expressing the amiRs under tissue-specific promoters.

A particular advantage of amiRs is that amiR can be expressed at lower levels compared to shRNAs using RNApol II promoters that allow high expression in target cells yet ensure efficient processing and no neuronal damage. Moreover, amiRs target a specific protein within the same family and are thus highly specific with respect to small molecule inhibitors. The amiRs according to the invention are usually single-stranded molecules. while the amiR precursor are usually in the form of an at least partially self-complementary molecule capable of forming double-stranded portions, e.g., stem- and loop-structures Typically, the targeting sequence in the amiR is at least 12 nucleotides to 28 nucleotides, 20 nucleotides to 26 nucleotides, about 18, 19, 20, 21, 22, 23, 24, 25, or 26 nucleotides. Preferably, the targeting sequence of the RNAi molecule is 21 bp long, although other lengths are also possible.

According to preferred embodiments of the invention, the therapeutic agent comprises at least one genetic silencer being an amiR, or a precursor or source thereof, or an equivalent thereof, targeting PRMT6, preferably targeting the any one of sequences SEQ ID NO: 3-53, more preferably any one of sequences SEQ ID NO: 18, 21 or 29, or transcripts thereof. Preferably, said amiR comprises or consists of a polynucleotide having sequence perfectly complementary to any one of sequences SEQ ID NO:3-53, more preferably to any one of sequences SEQ ID NO: 18, 21 or 29, or transcripts thereof. Most preferably, said amiR comprises or consists of sequence SEQ ID NO: 171-173, or equivalent thereof. Optionally, said therapeutic agent further comprises at least one inhibitor of LSD1, preferably said inhibitor being a genetic silencer or a small molecule, more preferably being a genetic silencer.

According to preferred embodiments of the invention, the therapeutic agent comprises at least one genetic silencer being an amiR, or a precursor or source thereof, or an equivalent thereof, targeting LSD1, preferably targeting any one of sequences SEQ ID NO: 54-138, more preferably any one of sequences SEQ ID NO: 68, 72 or 90, or transcripts thereof. Preferably, said amiR comprises or consists of a polynucleotide having sequence perfectly complementary to any one of sequences SEQ ID NO: 54-138, more preferably to any one of sequences SEQ ID NO: 68, 72 or 90, or transcripts thereof. Most preferably, said amiR comprises or consists of sequence SEQ ID NO: 174-176, or equivalent thereof. Optionally, said therapeutic agent further comprises at least one inhibitor of PRMT6, preferably said inhibitor being a genetic silencer or a small molecule, more preferably being a genetic silencer.

According to particularly preferred embodiments, the genetic silencer of the invention is an amiR source, or equivalent thereof, comprising a nucleic acid molecule encoding an amiR of interest or precursor thereof, flanked by nucleic acid structural regions (such as the hairpin loop) derived from a natural miR precursor. Accordingly, the genetic silencer of the invention is preferably an amiR precursor, or equivalent thereof, comprising the nucleic acid of the amiR of interest flanked by nucleic acid structural regions (such as the hairpin loop) derived from a natural miR precursor. Structural regions flanking the amiR of interest can be derived for example from a miR-155 precursor, as described in US20040053876. The skilled person would understand that the present invention is also directed to amiRs precursors or sources, having stem-loop structures derived from miRNAs different from miR-155 and would know how to design said amiRs precursors or sources. For instance, stem-loop structures can be derived from miR-30 precursor or source, or others.

According to preferred embodiments, the therapeutic agent comprises at least one genetic silencer being a precursor or source, or an equivalent thereof, of an amiR targeting PRMT6, more preferably, targeting any one of sequences SEQ ID NO: 3-53, most preferably any one of sequences SEQ ID NO: 18, 21 or 29, or transcripts thereof. Preferably, said amiR precursor or source thereof, or equivalent thereof, comprises or consists of a polynucleotide having sequence perfectly complementary to any one of sequences SEQ ID NO:3-53, more preferably to any one of sequences SEQ ID NO: 18, 21 or 29, or transcripts thereof.

Preferably, said amiR precursor or source thereof, comprises sequence SEQ ID NO: 171-173, or equivalent thereof, more preferably comprises or consists of sequence SEQ ID NO: 177-179, or equivalent thereof. Optionally, said therapeutic agent further comprises at least one inhibitor of LSD1, preferably said inhibitor being a genetic silencer or a small molecule, more preferably being a genetic silencer.

According to preferred embodiments of the invention, the therapeutic agent comprises at least one genetic silencer being a precursor or source, or an equivalent thereof, of an amiR targeting LSD1. Preferably, said genetic silencer is a precursor, or equivalent thereof, of an amiR targeting any one of sequences SEQ ID NO: 54-138, more preferably targeting any one of sequences SEQ ID NO: 68, 72 or 90, or transcripts thereof. Preferably, said amiR precursor or source thereof, or equivalent thereof, comprises a polynucleotide having sequence perfectly complementary to any one of sequences SEQ ID NO: 54-138, more preferably to any one of sequences SEQ ID NO: 68, 72 or 90, or transcripts thereof. Preferably, said amiR precursor or source thereof, comprises sequence SEQ ID NO: 174-176, or equivalent thereof, more preferably comprises or consists of sequence SEQ ID NO: 180-182, or equivalent thereof. Optionally, said therapeutic agent further comprises at least one inhibitor of PRMT6, preferably said inhibitor being a genetic silencer.

According to preferred embodiments of the invention, the therapeutic agent comprises at least one genetic silencer being a source, or an equivalent thereof, of an amiR or precursor thereof targeting PRMT6. Preferably, said genetic silencer is a source, or equivalent thereof, of an amiR or precursor thereof targeting any one of sequences SEQ ID NO: 3-53, more preferably one of sequences SEQ ID NO: 18, 21 or 29, or transcripts thereof. Preferably, said amiR source or equivalent thereof, comprises a polynucleotide having sequence perfectly complementary to any one of sequences SEQ ID NO: 3-53, more preferably to any one of sequences SEQ ID NO: 18, 21 or 29, or transcripts thereof. Preferably, said amiR source, comprises or consists of sequence SEQ ID NO: 183-185, or equivalent thereof. Optionally, said therapeutic agent further comprises at least one inhibitor of LSD1, preferably said inhibitor being a genetic silencer or a small molecule, more preferably being a genetic silencer.

According to preferred embodiments of the invention, the therapeutic agent comprises at least one genetic silencer being a source, or an equivalent thereof, of an amiR or precursor thereof targeting LSD1. Preferably, said genetic silencer is a source or an equivalent thereof, of an amiR or precursor thereof targeting any one of sequences SEQ ID NO: 54-138, more preferably any one of sequences SEQ ID NO: 68, 72 or 90, or transcripts thereof. Preferably, said amiR source, or equivalent thereof, comprises a polynucleotide having sequence perfectly complementary to any one of sequences SEQ ID NO: 54-138, more preferably to any one of sequences SEQ ID NO: 68, 72 or 90, or transcripts thereof. Preferably, said amiR source, comprises or consists of sequence SEQ ID NO: 186-188, or equivalent thereof. Optionally, said therapeutic agent further comprises at least one inhibitor of PRMT6, preferably said inhibitor being a genetic silencer.

The RNAi molecules, precursors or sources thereof, and equivalents thereof, can be made by any technique known to one of ordinary skill in the art, such as for example, chemical synthesis, enzymatic production or biological production, including methods involving recombinant DNA technology.

The RNAi molecules or precursors thereof are typically made by chemical synthesis. Preferably, the RNAi molecule is delivered to a cell as precursor or source of the RNAi molecule. The RNAi source is preferably produced by recombinant methods for producing nucleic acids in a cell, that are well known to those of skill in the art. These include the use of vectors, plasmids, cosmids, and other vehicles for delivering a nucleic acid to a cell, which may be the target cell or simply a host cell (to produce large quantities of the desired RNAi molecule).

According to preferred embodiments, the therapeutic agent can comprise at least one genetic silencer being a genome editing agent.

A “genome editing agent” is an agent comprising an engineered nuclease which can mediate targeted gene disruption. Such nucleases may be delivered to a target cell using vectors, such as viral or non-viral vectors.

Examples of nucleases suitable for genome editing include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405). Meganucleases (Silve, G. et al. (2011) Cur. Gene Ther. 11: 11-27) may also be employed as suitable nucleases for gene editing.

“CRISPR/Cas system” refers collectively to transcripts and other elements involved in the expression of, or directing the activity of, CRISPR-associated (“Cas”) genes, including sequences encoding a Cas gene and a guide RNA, wherein the guide RNA (gRNA or sgRNA) may be selected to enable a Cas domain to be targeted to a specific sequence (van der Oost et al. (2014) Nat. Rev. Microbiol. 12: 479-92). Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21), and are suitable for use in the invention.

According to preferred embodiments of the invention, the therapeutic agent comprises, or consists of, at least one genome editing agent comprising at least one nuclease targeting PRMT6 and/or at least one nuclease targeting LSD1; more preferably said at least one genome editing agent is a CRISPR/Cas system comprising at least one non-coding RNA molecule (guide RNA), comprising a guide sequence which binds, sequence-specifically, to PRMT6 and/or LSD1 gene, and a Cas protein (e.g., Cas9 or the like), with nuclease functionality.

According to particularly preferred embodiments the therapeutic agent comprises, or consists of at least one CRISPR/Cas system, comprising at least one gRNA comprising a guide sequence that is complementary to a target sequence in PRMT6 gene, and/or comprising at least one gRNA comprising a guide sequence that is complementary to a target sequence in LSD1 gene; the CRISPR/Cas system further comprising a nucleic acid encoding a site-directed Cas nuclease, preferably a Cas9 nuclease or a variant thereof.

According to most preferred embodiments, the therapeutic agent comprises, or consists of, at least one CRISPR/Cas system comprising at least one gRNA and at least one nucleic acid encoding a site-directed Cas nuclease, wherein said at least one gRNA comprises a guide sequence that is complementary to a target sequence in PRMT6 gene, wherein said guide sequence is selected from the group consisting of SEQ ID NO: 236-334, and/or wherein said at least one gRNA comprises a guide sequence that is complementary to a target sequence in LSD1 gene, wherein said guide sequence selected from the group consisting of SEQ ID NO: 335-431.

Preferred guide sequences of gRNA suitable for use in accordance with the present invention are provided in Table 1 that follows; the PAM sequences cleaved in the target genes are also provided for each gRNA.

TABLE 1
gRNAs

| gRNA | | PRMT 6 |
|---|---|---|
| guide sequence | SEQ | gene PAM |
| targeting PRMT6 | ID NO: | Sequence |
| GCUGUACUACGAGUGCUACU | 236 | CGG |
| GGCAAGACGGUACUGGACGU | 237 | GGG |
| ACUGCGAGGCAAGACGGUAC | 238 | TGG |
| GAUACCCAGGCGGUAGGCAU | 239 | CGG |
| GGUGCAAGUGGAGCAAGACA | 240 | CGG |
| CAGAUGGCAAAGCCAUGCAU | 241 | GGG |
| UAUGGCUCGGCGCCCAUGCA | 242 | TGG |
| AGCCAGGUGAAGCAGCACUA | 243 | TGG |
| GCCCAUCCACUCGCUCACGA | 244 | TGG |
| UCCUCCACGCGCGAACCAAG | 245 | TGG |
| GGCAGCACUGCGAGGCAAGA | 246 | CGG |
| GCCAUCGUGAGCGAGUGGAU | 247 | GGG |
| CCGAAGGAUACCCAGGCGGU | 248 | AGG |
| GGCAACUCUACAGUCUCCAC | 249 | TGG |
| GGGGGCUAUGAAGAGCUCGG | 250 | CGG |
| AUCGUUGUGCAGGGAUUGUC | 251 | CGG |
| CACUCGGAGAUCGUUGUGCA | 252 | GGG |
| UGUGCAGGGAUUGUCCGGCG | 253 | AGG |
| ACGAUGGCAUCCACCUGUUC | 254 | CGG |
| GUGGCCCAUGAGACAGCGCG | 255 | TGG |
| GCGACCCCGGAGGACUAAGC | 256 | GGG |
| CCAGGCGGUAGGCAUCGGUG | 257 | CGG |
| UGGAUGCCAUCGUGAGCGAG | 258 | TGG |
| CGGUAGAGGCCAGCGCCAUC | 259 | TGG |
| GCAGUGCUGCCCAGUUCCGA | 260 | AGG |
| CCCAGUUCCGAAGGAUACCC | 261 | AGG |
| CCUACCGCCUGGGUAUCCUU | 262 | CGG |
| CCUGGGUAUCCUUCGGAACU | 263 | GGG |
| CGCACCGAUGCCUACCGCCU | 264 | GGG |
| CGUUCCCGCUUAGUCCUCCG | 265 | GGG |
| UCAGCCACUUGGUUCGCGCG | 266 | TGG |
| GGCUAUGAAGAGCUCGGCGG | 267 | AGG |
| ACAGAAGAUGCUCAGAAUGC | 268 | CGG |
| GUGAGCGAGUGGAUGGGCUA | 269 | CGG |
| CCUGCACAACGAUCUCCGAG | 270 | TGG |
| UCAGCGACCAGAUGCUGGAA | 271 | TGG |
| ACGGUACUGGACGUGGGCGC | 272 | GGG |
| AGCGCGUGGCGAAGCCCUCC | 273 | AGG |
| CCCGGCCACUCACUGGAAAC | 274 | AGG |
| GGGAGGUGGUGCGGUUCAAC | 275 | GGG |
| GGCCGGGUGAAAAGGCGAGG | 276 | TGG |
| UCUACAGUCUCCACUGGUCC | 277 | CGG |
| CAGCUAGAGCUCUCCCGCGC | 278 | CGG |
| GUUUCUCCGACUCCCCUCCA | 279 | GGG |
| CCGGCGCGUGUACGCGGUAG | 280 | AGG |
| GUGGAGGACGGAGCUCAGCA | 281 | TGG |
| GUGUCUUGCUCCACUUGCAC | 282 | CGG |
| AGUCCUCCGGGGUCGCUCCA | 283 | GGG |
| UCUAGCUGAGCAAAGCGCUG | 284 | CGG |
| GUGGACAUGAGCUGCCUGGA | 285 | GGG |
| CCAGAUGGCAAAGCCAUGCA | 286 | TGG |
| CGAACCAAGUGGCUGAAGGA | 287 | GGG |
| GUUGAACCGCACCACCUCCC | 288 | GGG |
| AAGGCGAGGUGGACAGCACC | 289 | AGG |
| GCGCUGUCUCAUGGGCCACU | 290 | CGG |
| AUGAAGAGCUCGGCGGAGGC | 291 | CGG |
| GGCCAGCGCCAUCUGGCAAC | 292 | AGG |
| CAGGCGACGGGGGUUGUCCC | 293 | GGG |
| CAGGAGCUGGAGGCCGGAGU | 294 | GGG |
| UCUGAGCAUCUUCUGUGCCC | 295 | AGG |
| CGGAACUGGGCAGCACUGCG | 296 | AGG |
| AGGCGAGGUGGACAGCACCA | 297 | GGG |
| CGGGGCCCGGCGCGUGUACG | 298 | CGG |
| GGUCCACGAGGAGAUGAUCG | 299 | CGG |
| CCAGGCGCCAUUCCAGCAUC | 300 | TGG |
| CCCCAUCAGCGACCAGAUGC | 301 | TGG |
| AGUGGAGACUGUAGAGUUGC | 302 | CGG |
| GCAGCACGCGCAGGCGACGG | 303 | GGG |
| AGGGGAGUCGGAGAAACCCC | 304 | TGG |
| UCACCUGGCUCCAGAAGCCC | 305 | AGG |
| GAAGACCAAAGACUUUGCCA | 306 | TGG |
| GGUGAAGCAGCACUAUGGUG | 307 | TGG |
| UGUAGCGCAGCAGCACGCGC | 308 | AGG |
| CUGGACGUGGGCGCGGGCAC | 309 | CGG |
| GCGCUACAAAGUGGGAGACC | 310 | AGG |
| GUGCUGCUGCGCUACAAAGU | 311 | GGG |
| GCGAACCAAGUGGCUGAAGG | 312 | AGG |
| GGCGAGGUGGACAGCACCAG | 313 | GGG |
| CCAGGUGACCUUCCCUGGAG | 314 | GGG |
| GAAGGUCACCUGGAACCAGA | 315 | TGG |
| AGUUCCGAAGGAUACCCAGG | 316 | CGG |
| GCAGCAGCACGCGCAGGCGA | 317 | CGG |
| CCGAGCCAUAGCAGCUGCAG | 318 | CGG |
| GUUCAACGGGCUGGAGGACC | 319 | GGG |
| GGUCCGCGAUCAUCUCCUCG | 320 | TGG |
| UGUGGACAUGAGCUGCCUGG | 321 | AGG |
| AGCUCAGCAUGGACUCGUGC | 322 | AGG |
| ACCGCCCUCCUUCAGCCACU | 323 | TGG |
| AUUGUCCGGCGAGGACGUGC | 324 | TGG |
| CCGGGCCAGCACGUCCUCGC | 325 | CGG |
| AGGCAACGCUCAGUCCUCCA | 326 | TGG |
| CUCCAGCUCCUGCUCCAAGC | 327 | CGG |
| GCAGGAGCUGGAGGCCGGAG | 328 | TGG |
| CAGCAUCUGGUCGCUGAUGG | 329 | GGG |
| GAUGGGGGCUAUGAAGAGCU | 330 | CGG |
| ACCAAGUGGCUGAAGGAGGG | 331 | CGG |
| UUGGAGCAGGAGCUGGAGGC | 332 | CGG |
| GAGGGAACUGAAGAGGAAGA | 333 | TGG |
| UUCCAGGUGACCUUCCCUGG | 334 | AGG |
| gRNA | | LSD1 gene |
| guide sequence | SEQ | PAM |
| targeting LSD1 | ID NO: | Sequence |
| AGUGCGACAGGUUCGCUACA | 335 | CGG |
| UGUGGUCCACUGAUAAUAUC | 336 | TGG |
| GGAUACUACAACUCACCCGA | 337 | TGG |
| UGGUAGCAGUGCAGUACCUC | 338 | AGG |
| UAUAAGGUGCUUCUAAUUGU | 339 | TGG |
| GCCACUAGUGCCAACAGUAU | 340 | TGG |
| GGAUCUGACCGCCCUAUGCA | 341 | AGG |
| UGUAUACCACACCUUGCAUA | 342 | GGG |
| AAGAUGUAGCUUCUAGCAAC | 343 | CGG |
| UAGGGCAAGCUACCUUGUUA | 344 | AGG |
| UCUUAGUGAAAAGCAAACAC | 345 | AGG |
| UUUCUGAAACAGGAUCGUGU | 346 | GGG |
| AUGUAUACCACACCUUGCAU | 347 | AGG |
| ACAUCUGCAGUCCAAAGGAU | 348 | GGG |
| GGGACACAGGCUUAUUAUUG | 349 | AGG |
| AUAAGUGACGAUGUGAUUGU | 350 | TGG |
| GGGAUUUGGCAACCUUAACA | 351 | AGG |
| GCAGUCCAAAGGAUGGGAUU | 352 | TGG |
| AGUGCCAACAGUAUUGGAGC | 353 | TGG |
| UCAACUUCGGCAUCUAUAAG | 354 | AGG |
| AGAUGAUCCUGCAGCAACAU | 355 | AGG |
| AGUAGCCAUUCCUUACUGUC | 356 | AGG |
| ACACACUACUUACUGUUAUA | 357 | AGG |
| AAGAUGAGCAGAUUGAACAU | 358 | TGG |
| GAACUCGGCAGUAAUAUCUC | 359 | TGG |
| AACAUCUGCAGUCCAAAGGA | 360 | TGG |
| UGGUAGAGCAAGAGUUUAAC | 361 | CGG |
| GAAUAAGAGCCCCGAGCCCA | 362 | GGG |
| AUAAAUAAAGGUUUGACUCG | 363 | TGG |
| UGACCGCCCUAUGCAAGGUG | 364 | TGG |
| UCGGCAGUAAUAUCUCUGGG | 365 | TGG |
| GGUCAGCAAACAAGUAAAUA | 366 | TGG |
| AAGGUAAUUAUUAUAGGCUC | 367 | TGG |
| GAAGGCAUUUACCUUUAUAG | 368 | AGG |
| AAGAAACUGUGGUGUCUCGU | 369 | TGG |
| ACCAAGACCUGUUACCACCA | 370 | TGG |
| AUACUGUUGGCACUAGUGGC | 371 | AGG |
| UGCCCGAACAAAUUGACACU | 372 | TGG |
| AGGAGAAGCUGCUGGUAUCA | 373 | TGG |
| AGGUAAUUAUUAUAGGCUCU | 374 | GGG |
| AGUGAGCCUGAAGAACCAUC | 375 | GGG |
| AGGAGGUCCUUACUUGGUAG | 376 | GGG |
| AAGUGAGCCUGAAGAACCAU | 377 | CGG |
| UUCAAGACGACAGUUCUGGA | 378 | GGG |
| AUAGGUAGAGUACAGAGAGA | 379 | TGG |
| UGAAAUGGAAACUUACUGUG | 380 | CGG |
| AAUGCCUGGCCAAGGGACAC | 381 | AGG |
| GGAAAACAUCUGCAGUCCAA | 382 | AGG |
| GGCGCAAGCGGGCGAAGGUA | 383 | AGG |
| GGUCAUGAGGAAGUCGGCUC | 384 | TGG |
| UCGUUGGCGUGCUGAUCCCU | 385 | GGG |
| GAGAGGUGGCACAAACUGAA | 386 | CGG |
| CAAACAAGUAAAUAUGGAAC | 387 | TGG |
| UCGACUUACAGCUUGUCCGU | 388 | TGG |
| CUUAGUGAAAAGCAAACACA | 389 | GGG |
| AGGAGGGAAUCCUAUGGCUG | 390 | TGG |
| AACUCGGCAGUAAUAUCUCU | 391 | GGG |
| AGUUGGAAGCGAAUCCCCCA | 392 | AGG |
| AGAGCCGACUUCCUCAUGAC | 393 | CGG |
| GAGCACCAUGCACUGUGGCU | 394 | GGG |
| UGGACCACAACAGACCCAGA | 395 | AGG |
| AGGCCUUCUGCUAAAGCCAC | 396 | AGG |
| CUCUUCUGGAACCUCUAUAA | 397 | AGG |
| UAGGAGGUCCUUACUUGGUA | 398 | GGG |
| AUACCACACCUUGCAUAGGG | 399 | CGG |
| ACUGAUAAUAUCUGGAAAAC | 400 | AGG |
| AGCCACCUGACAGUAAGGAA | 401 | TGG |
| UCAUCCGGUCAUGAGGAAGU | 402 | CGG |
| GGUGGCACAAACUGAACGGC | 403 | TGG |
| GAUGCCUGGCCAUUCUCAAA | 404 | GGG |
| AAUAAUACUCAUCUUCUGAG | 405 | AGG |
| AGUUCUCUGUACCCUUCCCC | 406 | TGG |
| CUGCUUCUUGAGAAGUCAUC | 407 | CGG |
| GAUGAUGACUUUGAGUUCAC | 408 | TGG |
| UGUGUCCCUUGGCCAGGCAU | 409 | TGG |
| AAUGCAGCUGCAGUUGUGGU | 410 | TGG |
| AGCUGAUCUUGGAGCCAUGG | 411 | TGG |
| GCUUCAGCACACCCAGGGGA | 412 | AGG |
| AACUUCCAAUGCCUGGCCAA | 413 | GGG |
| GUAGGAGGUCCUUACUUGGU | 414 | AGG |
| GGAAUAGCAGAGACUCCGGA | 415 | GGG |
| CCGCAAGAAAGAGCCUCCGC | 416 | GGG |
| GCUGCUGCUUCAGCACACCC | 417 | AGG |
| UGGUUAAAAGGACACUGUCA | 418 | AGG |
| GAGACAGACAAAUACUUGAU | 419 | TGG |
| GUGCCUGUGGCUUUAGCAGA | 420 | AGG |
| CUUCAGCACACCCAGGGGAA | 421 | GGG |
| GUUCUCUGUACCCUUCCCCU | 422 | GGG |
| UGCUGCUUCAGCACACCCAG | 423 | GGG |
| GAAGAAGAUAGUGAAAACUC | 424 | AGG |
| AGUUCUGGAGGGUAUGGAGA | 425 | CGG |
| CUGCUGCUUCAGCACACCCA | 426 | GGG |
| UCCUCAGGUGGGGCUUGAGG | 427 | GGG |
| GAAUAGCAGAGACUCCGGAG | 428 | GGG |
| GAAGCGAAUCCCCCAAGGUA | 429 | AGG |
| AGGCUCUGGGGUCUCAGGCU | 430 | TGG |
| AAGUCCUCCUGCUUGCCCUG | 431 | TGG |

According to preferred embodiments, the therapeutic agent comprises at least one genetic silencer being an antisense oligonucleotide (ASO) targeting PRMT6 and/or at least one genetic silencer being an ASO targeting LSD1.

An antisense oligonucleotide useful according to the invention may be RNA or DNA oligonucleotide.

Preferably, the ASO, as disclosed herein, is produced synthetically or recombinantly.

Preferably, an ASO is 10 to 22 nucleotides in length, more preferably 12 to 20, most preferably 14 to 18 nucleotides in length.

Preferably, the ASO, as disclosed herein, is about 15, 16, 17, 18, 19 or 20 nucleotides in length.

Preferably, the ASO, as disclosed herein, comprises at least one nucleoside analogue, e.g., a locked nucleic acid (LNA) monomer.

Preferably, the ASO, as disclosed herein, comprises at least one modified internucleoside linkage. The modified internucleoside linkage may be a peptide-nucleic acid linkage, a morpholino linkage, a N3′ to P5′ phosphoramidate linkage, a methylphosphonate linkage or a phosphorothioate linkage. More preferably, the ASO, as disclosed herein, comprises at least one modified internucleoside linkage being a phosphorothioate linkage.

Preferably, the ASO, as described herein, comprises a gapmer oligonucleotide consisting of 10 to 22 linked nucleosides, preferably 12 to 20 nucleosides, more preferably 15, 16, 17, 18 or 19 or 20 nucleosides, wherein the gapmer oligonucleotide has a 5′ wing region positioned at the 5′ end of a deoxynucleotide gap, and a 3′ wing region positioned at the 3′ end of the deoxynucleotide gap, wherein at least one nucleoside of at least one of the wing regions is a LNA, more preferably 2 to 4 nucleosides, most preferably 3 nucleosides are LNAs.

According to preferred embodiments, the therapeutic agent comprises at least one genetic silencer targeting PRMT6 being an ASO, more preferably wherein said ASO targets sequence SEQ ID NO: 432 or 433 of PRMT6.

Preferably, said ASO targeting PRMT6 has sequence comprising, or consisting of, SEQ ID NO: 440, or 441, or equivalents thereof. Preferably said ASO targeting PRMT6 is an ASO having sequence SEQ ID NO: 436, or 437 According to preferred embodiments, the therapeutic agent comprises at least one genetic silencer targeting LSD1 being an ASO, more preferably wherein said ASO targets sequence SEQ ID NO: 434 or 435 of LSD1.

Preferably, said ASO targeting LSD1 has sequence comprising, or consisting of, SEQ ID NO: 442, or 443, or equivalents thereof. Preferably said ASO targeting PRMT6 is an ASO having sequence SEQ ID NO: 438, or 439.

Preferred ASOs and their targets are shown in the table that follows, wherein:
+ indicates an LNA and s indicates a phosphorothioate (PS) backbone.

| | Target sequence in | |
|---|---|---|
| ID | PRMT6 | ASO sequence |
| ASO 1 | TGTACTACGAGTGCTA | +Ts+As+GsCsAsCsTsCsGsTsAsGsTs+As+Cs+A |
| | (SEQ ID NO: 432) | (SEQ ID NO: 436) |
| ASO 2 | TGCACGAGTCCATGCT | +As+Gs+CsAsTsGsGsAsCsTsCsGsTs+Gs+Cs+A′ |
| | (SEQ ID NO: 433) | (SEQ ID NO: 437) |
| | Target sequence in | |
| ID | LSD1 | ASO sequence |
| ASO 3 | TACTGTGCTTGTCCAC | +Gs+Ts+GsGsAsCsAsAsGsCsAsCsAs+Gs+Ts+A |
| | (SEQ ID NO: 434) | (SEQ ID NO: 438) |
| ASO 4 | CTATGTAGCTGATCTTG | +Cs+As+AsGsAsTsCsAsGsCsTsAsCsAs+Ts+As+G |
| | (SEQ ID NO: 435) | (SEQ ID NO: 439) |

According to preferred aspects, the present invention is directed to a therapeutic agent comprising at least one inhibitor of PRMT6 and at least one inhibitor of LSD1; more preferably said at least one inhibitor of PRMT6 and/or said at least one inhibitor of LSD1 is a genetic silencer targeting PRMT6 and/or LSD1.

Preferably, the therapeutic agent comprising at least one genetic silencer targeting LSD1, optionally further comprising at least one inhibitor of PRMT6, is for use in the treatment of SBMA or of a cancer, more preferably of SBMA.

Preferably, the therapeutic agent comprising at least one genetic silencer targeting PRMT6, optionally further comprising at least one inhibitor of LSD1, is for use in the treatment of a cancer or of SBMA, more preferably of prostate cancer.

Preferably, the therapeutic agent comprises at least two genetic silencers, more preferably at least two RNAi molecules or at least two gene editing agents, wherein at least one is targeting PRMT6 and at least one is targeting LSD1.

More preferably, the therapeutic agent of the invention comprises at least one genetic silencer targeting LSD1 and at least one inhibitor of PRMT6, said therapeutic agent being for use in the treatment of SBMA or of a cancer, most preferably of SBMA.

Optionally, the therapeutic agent of the invention comprises at least one genetic silencer targeting LSD1, at least one genetic silencer of PRMT6 and at least one small molecule inhibitor of LSD1 and/or of PRMT6, preferably said therapeutic agent being for use in the treatment of SBMA or of a cancer, more preferably of SBMA.

More preferably, the therapeutic agent of the invention comprises at least one genetic silencer targeting PRMT6 and at least one inhibitor of LSD1, said therapeutic agent being for use in the treatment of SBMA or of a cancer, more preferably of cancer.

Optionally, the therapeutic agent of the invention comprises at least one genetic silencer targeting LSD1, at least one genetic silencer of PRMT6 and at least one small molecule inhibitor of LSD1 and/or of PRMT6, preferably said therapeutic agent being for use in the treatment of SBMA or of a cancer, more preferably of cancer.

According to preferred aspects of the present invention, the therapeutic agent of the invention comprises at least one genetic silencer of an AR coactivator and further comprises a delivery vehicle for delivering said genetic silencer in a cell, preferably said delivery vehicle being selected from a viral vector, microspheres, liposomes, nanoparticles, microparticles, colloidal gold particles, lipopolysaccharides, polypeptides, polysaccharides, collagen, pegylation of viral vehicles, graphene composites, cholesterol conjugates, cyclodextran complexes, or polyethyleneimine polymers. Preferably, said delivery vehicle is a viral vector, more preferably selected from: an adeno-associated viral vector, a lentiviral vector, an adenoviral vector, a retroviral vector, an alphaviral vector, a vaccinia virus vector, a herpes simplex virus (HSV) vector, a rabies virus vector, and a Sindbis virus vector.

According to preferred embodiments, the therapeutic agent comprises at least one RNAi molecule source and a delivery vehicle, comprising the RNAi molecule source; more preferably the delivery vehicle is a viral vector, most preferably a recombinant adeno-associated viral vector (“recombinant AAV”).

The recombinant AAV is a viral particle containing two elements, an AAV capsid and a vector genome comprising non-AAV coding sequences packaged within the AAV capsid. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrhlO, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9(hul4), AAV 10, AAV 11, AAV 12, AAVrh8, AAVrh74, AAV-DJ8, AAV-DJ, AAVhu68, without limitation; most preferably, the viral vector is a AAV9 recombinant viral vector.

Advantageously, it is possible to clone more than one source of genetic silencers in the same vector, which allows to use the same amount of virus to target two genes.

Production of viral particle for delivering a genetic silencer according to the invention can be carried out by techniques known in the art (See, e.g., WO 2003/042397; WO 2005/033321, WO 2006/110689; U.S. Pat. No. 7,588,772 B2).

Preferably, the vector for delivering the genetic silencer in a cell is a non-viral plasmid that comprises an expression cassette for expressing the genetic silencer. Optionally the plasmid or other nucleic acid sequence is delivered via a suitable device, e.g., via electrospray, electroporation. In other embodiments, the genetic silencer is coupled with various compositions and nano particles, including, e.g., gold or silica nanoparticles, lipids, polymers, micelles, liposomes, exosomes, cationic lipid-nucleic acid compositions (lipoplexes), poly-glycan compositions and other polymers, lipid and/or cholesterol-based-nucleic acid conjugates, and others. Most preferred, for delivering the genetic silencer in a cell, are biocompatible lipoplexes, such as those described in Nicoletti et al. 20225.

Preferably, a RNAi molecule or precursor thereof, or equivalent thereof, is encapsulated in a nanoparticle, more preferably in a lipid nanoparticle (LNP). As used herein, the terms “lipid nanoparticle” refer to a transfer vehicle comprising one or more lipids (e.g., cationic lipids, non-cationic lipids, and PEG-modified lipids). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkylcyanoacrylates, polylactide, polylactide-polyglycolide copolymers, polycaprolactones, dextran, albumin, gelatin, alginate, collagen, chitosan, cyclodextrins, dendrimers and polyethyleneimine.

Useful lipid nanoparticles for RNA comprise a cationic lipid to encapsulate and/or enhance the delivery of a RNAi molecule or precursor thereof into the target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. LNP formulation can be performed using routine procedures comprising cholesterol, ionizable lipid, helper lipid, PEG-lipid and polymer forming a lipid bilayer around encapsulated mRNA. Preferably, LNP comprises a cationic lipid (i.e., N-[1-(2,3-dioleoyloxy) propyl]-N, N, N-trimethylammonium chloride (DOTMA), or 1,2-dioleoyl-3-trimethylammonium-propane (DOTAP)) with helper lipid DOPE, or an ionizable lipid Dlin-MC3-DMA ionizable lipids, or diketopiperazine-based ionizable lipids (cKK-E12). Preferably, polymer comprises a polyethyleneimine (PEI), or a poly(-amino) ester (PBAEs).

According to preferred embodiments of the invention, the therapeutic agent comprises at least one small molecule inhibitor of PRMT6 and/or LSD1; more preferably the therapeutic agent comprises at least one small molecule inhibitor of LSD1, being a monoamine oxidase inhibitor (MAOI), most preferably phenelzine, tranylcypromine, or mixtures thereof.

Optionally, the therapeutic agent of the invention comprises a small molecule inhibitor together with a genetic silencer inhibitor. Preferably, both the genetic silencer and the small molecule inhibitor inhibit PRMT6, or both the genetic silencer and the small molecule inhibitor inhibit LSD1; more preferably, the therapeutic agent comprises at least one small molecule inhibitor of LSD1 and at least one genetic silencer of PRMT6 and/or at least one genetic silencer of LSD1.

The therapeutic agent of the invention may be administered to a subject alone or in the form of a pharmaceutical formulation comprising one or more physiologically acceptable carriers, diluents, or excipients.

The term “pharmaceutically (or “physiologically”) acceptable diluents, or excipients” refers to a non-toxic solid, semisolid, or liquid filler, diluent, encapsulating material, or formulation auxiliary of any conventional type that may optionally be included in the compositions of the invention and that causes no significant adverse toxicological effects to the patient. A pharmaceutically acceptable excipient is essentially non-toxic to recipients at the employed dosages and concentrations and is compatible with other ingredients of the formulation. The number and the nature of the pharmaceutically acceptable excipients depend on the desired administration form. Pharmaceutically acceptable excipients are known and may be prepared by methods well known in the art.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference).

Proper formulation is dependent upon the inhibitor to be administered and the route of administration. For example, when the therapeutic agent comprises both small molecule inhibitors and genetic silencers, each of those is administered with a different formulation to a subject in need thereof. For example, the small molecule inhibitors can be administered as solid formulations, while the genetic silencer can be administered as liquid formulation comprising a suitable vehicle for delivering the genetic silencer.

Systemic formulations include those designed for administration by injection, e.g., subcutaneous, intravenous, intramuscular, intrathecal or intraperitoneal injection, as well as those designed for transdermal, transmucosal, inhalation, oral or pulmonary administration. For injection, the therapeutic agent or pharmaceutical formulation of the invention may be formulated in aqueous solutions, preferably in physiologically compatible buffers such as Hanks' solution, Ringer's solution, or physiological saline buffer. The solution may contain formulating agents such as suspending, stabilizing and/or dispersing agents.

Alternatively, the therapeutic agent or pharmaceutical formulation may be in solid form or it can be in powder form for constitution with a suitable vehicle, e.g., sterile pyrogen-free water, before use. For transmucosal administration, penetrants appropriate to the barrier to be permeated are used in the formulation. Such penetrants are generally known in the art.

For oral administration, e.g., for administering a therapeutic agent comprising a small molecule inhibitor, the therapeutic agent or pharmaceutical formulation can be readily formulated by combining the molecules with pharmaceutically acceptable carriers well known in the art. Such carriers enable the nucleic acids of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropyl methyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques. For oral liquid preparations such as, for example, suspensions, elixirs and solutions, suitable carriers, excipients or diluents include water, glycols, oils, alcohols, etc. Additionally, flavouring agents, preservatives, collaring agents and the like may be added. For buccal administration, the therapeutic agent or pharmaceutical formulation may take the form of tablets, lozenges, etc. formulated in conventional manner.

For administration by inhalation, the therapeutic agent or pharmaceutical formulation for use according to the present invention is conveniently delivered in the form of an aerosol spray from pressurized packs or a nebulizer, with the use of a suitable propellant. In addition to the formulations described previously, the therapeutic agent or pharmaceutical formulation for use according to the present invention may also be formulated as a depot preparation. Such long-acting preparations may be administered by implantation (for example subcutaneously or intramuscularly) or by intramuscular injection.

The therapeutic agent or pharmaceutical formulation according to the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularly, mucosally, intrapericardially, intraumbilically, intraocularally, by inhalation (e.g. aerosol inhalation), injection, infusion, continuous infusion, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Preferably, for administration to a human subject in need thereof, the therapeutic agent comprising a vector for delivering a genetic silencer is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, or a pH of 7.2 to 7.4, for intrathecal delivery, a pH within this range may be desired; whereas for intravenous delivery, a pH of about 6.8 to about 7.2 may be desired. However, other pHs within the broadest ranges and these subranges may be selected for other route of delivery.

Preferably, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Preferably, the formulation may contain, in addition to a vector (e.g., rAAV) and carrier(s), other conventional pharmaceutical ingredients, such as preservatives, or chemical stabilizers.

The therapeutic agent comprising a vector for delivering a genetic silencer is administered in sufficient amounts to provide to a cell a sufficient level of the genetic silencer to provide a therapeutic benefit without undue adverse effects, or with medically acceptable physiological effects, which can be determined by those skilled in the medical arts. Dosages of a vector to be administered for delivering the PRMT6 and/or LSD1 inhibitor according to the invention will depend primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range.

The practitioner responsible for administration will, in any event, determine the concentration of active ingredient(s) in a composition and appropriate dose(s) for the individual subject.

The term “unit dosage form” or “unitary dose”, as used herein, refers to physically discrete units suitable as unitary dosages for human and animal subjects, each unit containing a predetermined quantity of the compound, composition or formulation to be administered, calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

Preferably, a unitary dose of a pharmaceutical formulations for administering the therapeutic agent may comprise, for example, at least about 0.1 wt. %, 1-90 wt. %, 2-75 wt. % 25-60 wt. %, of the therapeutic agent, based on the weight of the unitary dose, and any range derivable therein. In other non-limiting examples, a unitary dose may also comprise less than 1 μg/kg/body weight, or 1 μg/kg/body weight, from 5 μg/kg/body weight, 10 μg/kg/body weight, 50 μg/kg/body weight, 100 μg/kg/body weight, 200 μg/kg/body weight, 350 μg/kg/body weight, 500 μg/kg/body weight, 1 mg/kg/body weight, 5 mg/kg/body weight, 10 mg/kg/body weight, 50 mg/kg/body weight, 100 mg/kg/body weight, 200 mg/kg/body weight, 350 mg/kg/body weight, or 500 mg/kg/body weight, to 1000 mg/kg/body weight or more per administration, and any range derivable therein. In non-limiting examples of a derivable range from the numbers listed herein, a range of 5 mg/kg/body weight to 100 mg/kg/body weight, 5 μg/kg/body weight to 500 mg/kg/body weight, etc., can be administered, based on the numbers described above.

Preferably, a pharmaceutical formulation according to the invention comprising at least one small molecule inhibitor is administered at a daily dose of 1-100 mg, preferably at a daily dose of 5-100 mg, 10-100 mg, or 15-100 mg.

For example, a pharmaceutical formulation comprising a MAOI can be administered at a dose of 15-30 mg, from once a day up to three times a day, preferably the administered dosage increasing during time.

As a further example, a pharmaceutical formulation comprising an ASO, as described herein, in the oral dosage form (e.g., a tablet or a capsule) comprises an amount of ASO in the range of about 1 mg to about 100 mg, about 5 mg to about 100 mg, about 10 mg to about 100 mg, about 20 mg to about 100 mg, or about 20 mg and about 50 mg.

Optionally, when the therapeutic agent comprises a combination of an inhibitor of LSD1 and an inhibitor of PRMT6, each inhibitor can be administered individually. Therapeutically effective levels of the therapeutic agent may thus be achieved by administering multiple doses each day. The amount of therapeutic agent administered will, of course, be dependent on the subject being treated, on the subject's weight, the severity of the affliction, the manner of administration and the judgment of the prescribing physician.

Preferably, a therapeutically effective dose of the molecules described herein will provide therapeutic benefit without causing substantial toxicity. Toxicity of the molecules described herein can be determined by standard pharmaceutical procedures in cell cultures or experimental animals, e.g., by determining the Maximal tolerated dose (Ann.Pharm, Fr, 2010, 291-300). The dose ratio between toxic and therapeutic effect is the therapeutic index. The data obtained from these cell culture assays and animal studies can be used in formulating a dosage range that is not toxic for use in human.

It should be understood that all the possible combinations of the preferred aspects of the present invention are also described, and therefore similarly preferred.

Examples of preferred embodiments of the present invention and analyses of their efficacy are provided below for illustrative and non-limiting purposes.

In particular, as shown in the following examples, the therapeutic agents according of the invention are capable to normalize the expression of AR coactivators in vivo. This is particularly important for LSD1, whose genetic ablation is lethal, but also in general since in the treated subjects the inhibited AR coactivators are capable of mainlining their physiological function. Advantageously, amiRs targeting AR coactivators exert a therapeutic effect in models of prostate cancer and SBMA.

Moreover, evidence is provided that targeting overexpressed AR co-regulators that enhance mutant AR toxic GOF modifies also expression of genes involved in pathways dysregulated in SBMA muscle and key to muscle physiology and homeostasis. These pathways include muscle contraction and myofibril assembly, glycolysis, and metabolism. Although the number of rescued genes is limited with respect to the total number of genes dysregulated in SBMA muscle, this effect is associated with amelioration of phenotype in a severe mouse model of SBMA. Furthermore, although the amiR is delivered systemically, down-regulation of LSD1 and PRMT6 is shown to be significant only in skeletal muscle, which is consistent with the idea that polyQ expansions in the AR induce transcription of these co-regulators, which in turn enhance AR function, so that means to block or attenuate this feedforward mechanism have effects on skeletal muscle. The fact the target gene silencing is not significant in other tissues is consistent with the idea that this pathogenetic mechanism specifically occurs in skeletal muscle.

### EXAMPLES

### Materials and Methods

### Animals and Treatments

Animal care protocols conform with the appropriate national legislation (art. 31, D.lgs. 26/2014) and guidelines of the Council of the European Communities (2010/63/UE) and were approved by local ethics committees (Universities of Trento and Padova, Italy) and the Italian Ministry of Health. AR100Q transgenic and AR113Q knock-in mice were genotyped. Mice were surgically castrated. Mice were subjected to intraperitoneal injection of either saline solution or amiR-Prmt6/Lsd1 AAVs at 21 days of age and were evaluated weekly during weeks 4-14. Mice were euthanized when they lost >20% of body weight with respect to the highest weight measurement. For rotarod and hanging wire tasks, mice were randomized, and both genotype and AAV injection were disguised to the operator. Animals were trained to run on an accelerated rotarod (4-40 rpm) (Panlab, Harvard apparatus, LE8205) for a maximum of 300 seconds. Latency to fall off was recorded, and the best performance of three trials was reported. For the hanging wire test, mice were placed on top of a wire cage lid, which was gently shaken three times to cause the mice to grip the wires, and then the lid was turned upside down. The latency to fall off—for a maximum of 60 seconds—was recorded. For survival analysis, moribundity was the time in which the mouse lost 20% of body weight or showed inability to move, dehydration, and cachexia.

All Drosophila stocks were maintained on standard cornmeal medium and fed 2 mM DHT at 28° C. in light/dark-controlled incubators. Lsd1 and Dart8 lines were obtained from the Vienna Drosophila Resource Center (VDRC, stock ID for DART8: v100228, and dLsd1: v106147). The AR52Q line was previously described². Eye images were taken with a Leica M205C dissection microscope equipped with a Leica DFC450 camera. Eye degeneration was quantified as previously described 2

### Human Samples

Deanonymized control (n=5) and patient biopsy samples (n=5) were obtained from the Neuromuscular Bank of Tissues and DNA Samples, Telethon Network of Genetic Biobanks, and EuroBioBank Network (Table 2). All muscle biopsies were taken for diagnostic purpose after written informed consent was obtained from each patient according to the Helsinki Declaration. All patients who underwent muscle biopsy were clinically affected and showed weakness and/or fasciculation and/or muscle atrophy. Myopathic changes together with neurogenic atrophy were observed in muscle biopsies. Control samples were obtained from subjects not suffering from any neurological and neurodegenerative condition. SBMA lumbar spinal cord tissue was a gift from Dr. Lyle Ostrow (ALS Postmortem Tissue Core at Johns Hopkins University).

TABLE 2
SBMA patient clinical information.

| | Age at | Disease | | CAG |
|---|---|---|---|---|
| | biopsy | onset | | repeat |
| | (year) | (year) | Muscle | length |
| Control | 37 | / | quadriceps femoris | / |
| Control | 42 | / | vastus lateralis | / |
| Control | 68 | / | vastus lateralis | / |
| Control | 45 | / | vastus lateralis | / |
| Control | 68 | / | vastus lateralis | / |
| SBMA | 54 | 60 | quadriceps femoris | 44 |
| SBMA | 51 | 48 | vastus lateralis | 44 |
| SBMA | 57 | 45 | vastus lateralis | 46 |
| SBMA | 64 | 57 | vastus lateralis | 42 |
| SBMA | 54 | 45 | vastus lateralis | 49 |

### Vectors

Transgenes to express shRNA against PRMT6 (shPRMT6 #1, SEQ ID NO: 232 and shPRMT6 #2, SEQ ID NO: 233) and the corresponding scramble were cloned into lentiviral constructs (pLKO.1-puro).

Guide RNAs were cloned into lentiCRISPR v1 (Addgene Plasmid 49535).

HEK293T cells were transfected by calcium phosphate with lentiviral vectors together with pCMV-dR8.91 (Delta 8.9) plasmids containing gag, pol, and rev genes and VSV-G envelope plasmid. At 16 hours post-transfection, medium was replaced with fresh medium, and 24 hours later it was collected, centrifuged at 1000×g for 10 minutes (to pellet and thus remove any cellular debris), filtered through 0.45-μm pores, and stored at −80° C. in aliquots. To quantify viruses, 10 μL of viral particles were lysed for 10 minutes at room temperature by adding an equal volume of 2× lysis buffer [0.25% Triton X-100, 50 mM KCl, 100 mM Tris-HCl pH 7.4, 40% glycerol, and 0.8 U/μL RNase inhibitor (RiboLock, Fermentas)]. Lysates were added to a single-step RT-PCR assay with 3.5 nM MS2 RNA (Roche) as template, 500 nM of each primer and hot-start Taq (Truestart Hotstart Taq, Fermentas), all in 20 mM Tris-Cl pH 8.3, 5 mM (NH₄)₂SO₄, 20 mM KCl, 5 mM MgCl₂, 0.1 mg/ml BSA, 1/20,000 SYBR Green I (Invitrogen, #S7563), and 200 M dNTPs. SG-PERT reverse transcription assay was carried out according with the following program: 42° C. for 20 minutes for reverse transcription reaction, 95° C. for 2 minutes for enzyme activation, followed by 40 cycles of denaturation at 95° C. for 5 seconds, annealing at 60° C. for 5 seconds, extension at 72° C. for 15 seconds, and acquisition at 80° C. for 5 seconds. A standard curve was obtained using known concentrations of high-titre viral supernatants (kindly provided by Dr. Massimo Pizzato, University of Trento, Italy). Lentiviruses were tested in vitro by transducing motor-neuron cell lines, and the most efficient knock-down was observed with shPRMT6 #1 (SEQ ID NO: 232). Mutagenesis of LSD1-LXXAA mutant was performed by Vector Builder (https://en.vectorbuilder.com/).

Cell Cultures, Transfection, and Transduction MN1⁹ and HEK293T (ATCC) cells were cultured and plated in DMEM complete medium [Dulbecco's Modified Eagle Medium supplemented with 10% fetal bovine serum (FBS), 1% penicillin/streptomycin (pen/strep), and 1% L-glutamine] and LNCaP cells in RPMI complete medium (Gibco Roswell Park Memorial Institute supplemented with 10% FBS, 1% penicillin/streptomycin, and 1% L-glutamine). C2C12 cells were cultured and differentiated to myotubes. Cells were kept in a humidified incubator with 5% CO₂ at 37° C. HEK293T cells were transfected with polyethyleneimine (PEI) linear MW 25,000 Da (Sigma-Aldrich) according to well dimensions. DNA: PEI (0.5% v/v) ratio was 1:1. At 24 hours after the transfection the DMEM complete medium supplemented with 10% FBS was changed with DMEM supplemented with charcoal stripped FBS and cells were treated with vehicle (ethanol) or 10 nM DHT and harvested for the different analysis 24 h after the treatment. For the pharmacological inhibition, we treated cells with the LSD1 catalytic inhibitor tranylcypromine (TCP) and the PRMT inhibitor adenosine dialdehyde (AdOx) both at a final concentration of 10 μM, instead for the selective LSD1 inhibitor SP-2509, we used it at a final concentration of 100 nm. MN-1 cells were transfected with Lipofectamine 2000 according to manufacturer instructions (Thermo Fisher). MN1 and LNCaP cells were transiently transduced with lentiviruses (MOI 30) or transfected with 2 μg DNA using Lipofectamine 2000 CD Transfection Reagent (ThermoFisher Scientific, 12566014). The following day, positively transfected cells were selected with 10 μg/L Blasticidin (PanReacApplichem, A3784,0025). After 24 hours of selection or at 48 hours post-transduction, cells were induced with 10 nM dihydrotestosterone (DHT) in DMEM or RPMI medium supplemented with 10% charcoal-stripped FBS, 1% pen/strep, and 1% L-glutamine. The SBMA iPSCs were derived and differentiated. iPSCs were maintained on Matrigel (Corning, 354277) coated tissue culture dishes in E8 Flex medium (Thermo Fisher, A2858501). The medium was changed every two days and the iPSCs were split every 4-6 days using accutase (StemCell Technology 07922). The culture medium was supplemented with 10 M ROCK inhibitor (Tocris, 1254) on the day of passaging. For differentiation stably transfected iPSCs were induced with Neural Induction Medium (NIM) containing 2 g/mL doxycycline and 10 nM R1881. After 48 hours of doxycycline treatment, motor neurons were dissociated with accutase to single cells and re-plated on PDL/Laminin coated surfaces in Neural Differentiation Medium (NDM) containing 2 g/mL doxycycline and 10 nM R1881. On day 4, half of the cell culture medium was removed and replaced by Neuron Medium (NM) containing 10 nM R1881.

### Immunocytochemistry

Immunofluorescence analysis in MN1 cells was performed. For IPSC-derived MNs, 6 dpi iMNs were washed with DPBS and fixed with 4% paraformaldehyde for 10 min at RT and washed again with DPBS before antibody labelling. Cells were first permeabilized with 0.1% Triton X-100 and 0.01% Tween-20 for 10 min at RT and then blocked with 10% BSA in 0.1% Triton X-100 and 0.1% Tween (PBST) for 1 hour at RT. Samples were then incubated overnight at 4° C. with primary antibodies in 3% BSA in PBST: anti-PRMT6 (Santa Cruz), anti-LSD1 (abcam), anti-HB9 (DSHB), and anti-AR (GeneTex). After overnight incubation, samples were washed twice with DPBS and incubated with fluorescent secondary antibodies (ThermoFisher) in 3% BSA in PBST for 1 hour at RT. The slides were mounted using ProLong Diamond Antifade Mountant with DAPI (ThermoFisher). For immunofluorescence analysis of post-mortem spinal cord tissue, frozen sections were fixed with 4% PFA and incubated overnight at 4° C. with antibodies as described above for anti-LSD1 (Abcam), anti-AR[H280](Santa Cruz), and DAPI. Digital images were captured with a Zeiss LSM 880 confocal microscope with a 40× objective.

### Cell Viability and Proximity Ligation Assay (PLA)

MN1 AR24Q and AR100Q cells were plated in a 24-well plate at 50000 cells/well confluence in DMEM complete, and 24 hours post-transfection DHT was added to the medium and the day after, MTT assay was performed. Briefly, MTT was directly added to the medium in a 1:10 ratio (50 μl/well) and left in the incubator with 5% CO₂ at 37° C. for 30 to 45 minutes until purple precipitates were visible in the plate. Medium was then replaced with 200 μL of dimethyl sulfoxide (DMSO) to dissolve the formazan product in a purple-coloured solution and the plate was kept in shaking for 10 minutes until all precipitate was completely dissolved. Finally, the solution was transferred to a 96-well plate and absorbance at 570 nM and 690 nM was quantified at Tecan Infinite® 200 PRO spectrophotometer. The final absorbance was obtained by subtracting the signal at 690 nM to the one at 570 nM. For PLA, cells were fixed with 4% PFA for 20 minutes, washed three times with 1×PBS, permeabilized with 0.1% Triton X-100 in PBS for 5 minutes, and proceeded for PLA analysis [Duolink In Situ Red Starter Kit Mouse/Rabbit, Sigma (Merck), DU092101]. The following primary antibodies were incubated overnight at 4° C.: anti-KDM1/LSD1 (Abcam, ab17721, 1:2000), anti-AR (AR 441, Santa Cruz Biotechnology, sc-7305, 1:2000), anti-PRMT6 (Bethyl, A300-929A, 1:2000), and anti-PRMT6 (Abcam, ab151191, 1:2000). Slides were imaged with a 63× oil immersion objective using the Zeiss Axio Observer Z1 inverted microscope, and PLA-positive red dots were quantified using ImageJ 1.51 software.

EdU Staining LNCaP cells were plated on coverslips pre-treated with poly-d-lysine in a 12-well plate at 160000 cells/well confluence in RPMI complete, and 24 hours post-transfection positively transfected cells were selected with 10 μg/L blasticidin (PanReacApplichem, A3784,0025). DHT was added to the medium and the day after, EdU assay was performed (Click-iT® Plus EdU Alexa Fluor 594 Imaging Kits, Invitrogen, C10639) firstly by incubating cells with 10 M EdU for 30 minutes. Then, media was changed with fresh RPMI complete and after 10 minutes cells were washed with PBS pre-warmed at 37° C. After that, cells were fixed with cold 4% PFA for 10 minutes, washed twice for 5 minutes with 3% BSA in PBS, permeabilized with 0.5% Triton X-100 in PBS for 20 minutes. Finally, cells were washed twice and Click-iT® Plus reaction cocktail was added to each coverslip for 30 minutes at room temperature and protected from light. Cells were then washed once with 3% BSA in PBS and with PBS for 5 minutes and the Hoechst® 33342 (Component G, 5 g/mL) was added for 30 minutes at room temperature, protected from light. Cells were washed twice with PBS and coverslips were mounted on slides. Slides were imaged with a 20× objective using the Zeiss Axio Observer Z1 inverted microscope, and EdU-positive red cells were quantified using ImageJ 1.51 software.

### Quantitative Real-Time PCR

Total RNA was extracted with TRIzol (Thermo Fisher Scientific), and RNA was reverse transcribed using Superscript Reverse Transcriptase III (Invitrogen, 18080093) following the manufacturer's instructions. Gene expression was measured by RT-qPCR using the SsoAdvanced Universal Sybr green supermix (1725274 Bio-Rad) and the C1000 Touch Thermal Cycler-CFX96 Real-Time System (Bio-Rad). Gene expression was normalized to Actin expression levels. The complete list of primer sequences is provided in Table 3.

TABLE 3
RT-PCR primers list

| Mouse | | |
|---|---|---|
| Genes | Forward | Reverse |
| Prmt6 | AGTCCATGCTGAGCTCCGT | TCCATGCAGCTCATATCCA |
| | (SEQ ID NO: 189) | (SEQ ID NO: 190) |
| Lsd1 | TGGAACTGGCCAAGATCAAG | GCTTCTAGCAACCGGTTAAATTC |
| | (SEQ ID NO: 191) | (SEQ ID NO: 192) |
| Actin | GACAGGATGCAGAAGGAGATTACTG | CTCAGGAGGAGCAATGATCTTGAT |
| | (SEQ ID NO: 193) | (SEQ ID NO: 194) |
| AR | GCCCGAATGCAAAGGTCTT | TGGCGTAACCTCCCTTGAAA |
| | (SEQ ID NO: 195) | (SEQ ID NO: 196) |
| Musk | ATCACCACGCCTCTTGAAAC | TGTCTTCCACGCTCAGAATC |
| | (SEQ ID NO: 197) | (SEQ ID NO: 198) |
| Myog | CTTGCTCAGCTCCCTCAAC | TGGGAGTTGCATTCACTGG |
| | (SEQ ID NO: 199) | (SEQ ID NO: 200) |
| Ncam | ACAATGCTGCGAACTAAGGA | TGCCACTTGACACAGGA |
| | (SEQ ID NO: 201) | (SEQ ID NO: 202) |
| Cdkn1a | AGGACCACGTGGCCTTGTC | TTTTCGGCCCTGAGATGTTC |
| | (SEQ ID NO: 203) | (SEQ ID NO: 204) |
| Fkbp4 | GACCGAGTCTTTGTCCACTACAC | ATCCCAAGCCTTGATGACCTCC |
| | (SEQ ID NO: 205) | (SEQ ID NO: 206) |
| Fkbp5 | AAAGGACAATGACTACTGATGAGG | CTGACAACATCCCTTTGTAGTGGAC |
| | (SEQ ID NO: 207) | (SEQ ID NO: 208) |
| Tp53 | GGGACGGGACAGCTTTGAG | AGGACTTCCTTTTTGCGGAAA |
| | (SEQ ID NO: 209) | (SEQ ID NO: 210) |
| Ezh2 | CATACGCTCTTCTGTCGACGATG | ACACTGTGGTCCACAAGGCTTG |
| | (SEQ ID NO: 211) | (SEQ ID NO: 212) |
| Human | | |
| Genes | Forward | Reverse |
| PRMT6 | GAGTGCTACTCGGACGTTTC | AGGATACCCAGGCGGTAG |
| | (SEQ ID NO: 213) | (SEQ ID NO: 214) |
| LSD1 | AGTGAGCCTGAAGAACCATC | TCTGTTGTGGTCCACTGATAAT |
| | (SEQ ID NO: 215) | (SEQ ID NO: 216) |
| AR | TGGGAGAGAGACAGCTTGTA | AGTACTGAATGACAGCCATCTG |
| | (SEQ ID NO: 217) | (SEQ ID NO: 218) |
| ATPA2 | TACCGAATTGAAGGGTCTTTCT | CGACATTGACTTTCTGTCACG |
| | (SEQ ID NO: 219) | (SEQ ID NO: 220) |
| CDKN1A | AAGACCATGTGGACCTGTCA | GGCTTCCTCTTGGAGAAGAT |
| | (SEQ ID NO: 221) | (SEQ ID NO: 222) |
| ACTIN | GGACTTCGAGCAAGAGATGG | AGCACTGTGTTGGCGTACAG |
| | (SEQ ID NO: 223) | (SEQ ID NO: 224) |

### RNA-Seq

Samples were subjected to RNA extraction with Trizol, following manufacturer's protocol. RNA was quantified using Nanodrop and Qubit and quality assessed by Agilent 2100 Bioanalyzer. Purified RNA served as input for the cDNA library preparation with TruSeq Stranded mRNA (Illumina), according to the manufacturer's protocol. The cDNA library fragment size was determined by the BioAnalyzer 2100 HS DNA Assay (Agilent, Santa Clara, CA, USA). The libraries were sequenced as paired-end reads on NovaSeq6000.

### Computational Analysis

For RNA-seq, data were analysed by Rosalind (https://rosalind.onramp.bio/), with a HyperScale architecture developed by Rosalind Inc. (San Diego, CA). Reads were trimmed using cutadapt (DOI:10.14806/ej.17.1.200). Quality scores were assessed using FastQC. Reads were aligned to the Mus musculus genome (mm10) using STAR (PMID:23104886). Individual sample reads were quantified using HTseq and normalized via relative log expression using DEseq2¹³, which also was used for differential expression analyses. P-values were adjusted for multiple hypothesis testing using the Benjamini and Hochberg method. Differentially expressed genes between non-treated (AR100Q) and treated (amiR-Lsd1/Prmt6) SBMA mouse models and WT controls were those genes with absolute fold-change >4 and adjusted p≤0.01. We defined a set of “rescued genes” as genes showing a significant but opposite direction in AR100Q vs WT and amiR-Lsd1/Prmt6 vs AR100Q differential expression analyses. Additionally, we identified a subset of rescued genes, referred to as “totally rescued”, showing no significant differential expression between amiR-Lsd1/Prmt6 and WT. The difference between rescued and totally rescued genes were the “partially rescued” genes. Functional enrichment analysis of rescued genes was performed using Metascape¹⁴, using the following gene lists: GO biological processes, KEGG pathway, and Reactome gene sets. All genes in the genome were used as enrichment background. Terms with p≤0.01, a minimum count of 3, and an enrichment factor >1.5 were collected and grouped into clusters based on membership similarities. The most statistically significant term within each cluster was chosen to represent the cluster. When comparing gene lists A and B using the overlapping coefficient measure, cardinality of the intersection between A and B was divided by minimum cardinality between cardinalities of A and B sets.

### Biochemistry

Cells were lysed for Western Blot analysis in RIPA buffer (6 mM Na2HPO4, 150 mM NaCl, 4 mM NaH2PO4, 150 mM NaCl, 2 mM EDTA pH 8.0, 1% Na-deoxycholate, 0.5% Triton X-100) plus fresh protease inhibitors (Sigma, P8340). Samples were incubated 20 minutes on ice and then centrifuged for 15 minutes at 21,000×g at 4° C. Supernatant was collected and stored at −80° C. or further processed for western blotting.

Frozen tissues were pulverized using a mortar and pestle on dry ice, transferred to a cold Eppendorf, resuspended in 2% SDS-RIPA buffer (50 mM Tris-HCl pH 8.0, 150 mM NaCl, 1% NP40, 0.5% Na-deoxycholate, 2% SDS) with fresh protease inhibitors (Sigma, P8340). Lysates were then sonicated and centrifuged at 15,000 rpm for 15 min at room temperature (RT).

The protein concentration was measured using the bicinchoninic acid (BCA) assay method (Pierce™ BCA® Protein Assay, Thermo Scientific). For Western blotting, equal amounts of protein extracts from tissues or cell lysates were boiled at 95° C. for 5 minutes in 5× sample buffer (62.5 mM Tris-HCl, pH 6.8, 2% SDS, 25% glycerol, 0.05% bromophenol blue, 5% β-mercaptoethanol) and separated by SDS-PAGE. Proteins were transferred to 0.45-mm nitrocellulose membranes (Bio-Rad, 162-0115), blocked for 1 hour in 5% non-fat milk/bovine serum albumin (BSA) in TBS buffer/0.1% Tween, and incubated with primary antibodies for 2 hours at room temperature or overnight at 4° C. HRP-conjugated secondary antibodies were incubated for 1 hour at room temperature (1:5,000 dilution in blocking solution), and signals were detected with Chemidoc (Bio-Rad) or Alliance Mini (Uvitec). For immunoprecipitation assays, cells were washed twice with ice-cold PBS, scraped, collected, and spun at 1,200×g for 3 minutes at 4° C. Pellets were lysed in IP buffer (50 mM HEPES, 250 mM NaCl, 5 mM EDTA, 0.1% NP40) containing 1 mM PMSF and fresh protease inhibitors (Sigma, P8340). Cell pellets were homogenized using syringes of 2.5 mL-22Gx1 ¼″ and 1 mL-25Gx⅝″, incubated on ice for 45 minutes, and centrifuged at 21,000×g for 30 minutes at 4° C. The supernatant was transferred and quantified by the Pierce™ BCA® Protein Assay (Thermo Scientific). Protein extract (1.5-4 mg) was incubated with primary antibodies overnight at 4° C. on a rotator. Then, the complex was incubated with protein A/G plus-agarose (sc-2003) beads for 2 hours at 4° C. on a rotator. Antigen-antibody complexes were washed three times with lysis buffer and once with wash buffer. Bound protein was eluted with 35 μL of 2×SDS buffer and denatured at 95° C. for 5 minutes before loading on SDS-PAGE.

For immunoprecipitation assays in tissue, quadriceps muscles were lysed in IP buffer (50 mM HEPES, 250 mM NaCl, 5 mM EDTA, 0.1% NP40) with fresh protease inhibitors (Sigma, P8340), incubated on ice for 30 minutes and centrifuged at 21,000×g for 45 minutes at 4° C. The supernatant was transferred and quantified by the Pierce™ BCA® Protein Assay (Thermo Scientific). Protein extract (2 mg) was pre-cleared with prewashed Pierce™ Protein A Magnetic Beads (30 uL, Thermo Fisher Scientific, cat #88845). for one hour at 4 degrees. Then beads were discarded and the pre-cleared lysate was incubated with 2 μg of primary antibody (PRMT6, Bethyl, A300-929A) or nonimmune rabbit immunoglobulin G (IgG) overnight at 4° C. on a rotator. The day after the antigen-antibody complexes were incubated with new pre-washed Pierce™ Protein A Magnetic Beads (20 uL, Thermo Fisher Scientific, cat #88845) for two hours at 4° C. on a rotator. Antigen-antibody complexes were washed four times with lysis buffer. Bound protein was eluted with 30 μL of NuPAGE™ LDS Sample Buffer (4×) and DTT 0.1 M and heated at 70° C. for 5 minutes before loading on a 10% SDS-PAGE gel.

The following antibodies were used for the different analysis: anti-KDM1/LSD1 (Abcam, ab17721, 1:1000), anti-PRMT6 (Proteintech, 15395-1-AP, 1:1000, ), anti-PRMT6 (Bethyl, A300-929A, 1:2000), anti-AR (for immunoprecipitation: 441, Santa Cruz, sc-7305), anti-AR (for immunoblot: H-280, Santa Cruz, sc-13062, 1:1000), anti-FLAG (Sigma, 7425), anti-GFP (Roche, 11814460001, 1:1000), anti-calnexin (Enzo, ADI-SPA-860, 1:2500), anti-tubulin (Sigma, T7816, 1:10000). Quantifications were performed using ImageJ 1.51 software.

Nicotinamide adenine dinucleotide (NADH) staining and analysis were carried out as previously described.

ChIP assays were performed, using 6×10⁷ C2C12 myoblasts expressing either AR24Q or AR100Q. AR was immunoprecipitated with anti-AR antibody (Millipore #06-680, 14 g, primer list is provided in Table 4).

TABLE 4
ChIP primers list.

| Primer | Primers (5′-3′) |
|---|---|
| Neg_Forward | TCCCTGTGCCCAAAGAGTAG |
| | (SEQ ID NO: 225) |
| Neg_Reverse | CCCTGAAGAATGAGGACGAG |
| | (SEQ ID NO: 226) |
| Lsd1_promoter_ARE_Forward | CAGTCTGGCCTACAAAGTGAG |
| | (SEQ ID NO: 227) |
| Lsd1_promoter_ARE_Reverse | GATTGGTTTTTCCGAGACAGG |
| | (SEQ ID NO: 228) |
| Prmt6_promoter_ARE_Forward | ACTTGTCATGGCGTTAGAGG |
| | (SEQ ID NO: 229) |
| Prmt6_promoter_ARE_Reverse | GATTCGTAGTCCGCTTCCTG |
| | (SEQ ID NO: 230) |

For histone purification, cells were seeded at a density of 7000 cells/cm2 and cultured in 10% FBS, and the cells were switched to the differentiation medium [DMEM, 2% horse serum, penicillin/streptomycin (100U/ml), and L-glutamine (2 mM)] when they reached 70-80% confluence at 37° C. in a humidified atmosphere containing 5% CO₂. The cells were replenished every three days. Histones were extracted from myocytes by the acid extraction method. Briefly, the cells were washed twice with ice-cold phosphate-buffered saline (PBS) and lysed in 1 ml (5×106 cells/ml) hypotonic lysis buffer (10 mM Tris-HCl pH 8.0, 1 mM KCl, 1.5 mM MgCl2, 1 mM DTT, 0.5 mM PMSF, 1× Protease and Phosphatase inhibitor) and incubated for 30 minutes on a rotator at 4° C. The nuclei were resuspended in 400 μl 0.4 N H₂SO₄ and incubated on ice for 30 minutes. The supernatant containing histones was precipitated with 132 μl of TCA on ice overnight. Following the centrifugation, the pellet was washed twice with 1 ml of ice-cold acetone and air-dried up. The histones were suspended in water and stored at −80° C. Histones were quantified by the colorimetric DC protein assay (Biorad, Cat. #5000111), according to the manufacturer's instructions, 5-10 μg of protein was used for further analysis.

Antibodies used: Anti-H3K3me2 (Ab7766) and anti-H3 (Ab1791).

Luciferase assays were performed. In brief, HEK293T cells were transfected with vectors expressing non-expanded and polyglutamine-expanded AR together with vectors expressing the luciferase reporter gene under the control of a canonical androgen responsive element (ARE-Luc). To normalize data for transfection efficiency, the cells express Renilla reporter gene under the control of the timidine kinase (TK-Ren) transfected with ARE-Luc in a ratio of 1:10. Cells are treated with vehicle and DHT for 16 h and then processed for luciferase assay, as manufacturer's instructions (Promega).

Generation of Artificial miRNAs (amiR) Targeting LSD1 and PRMT6

amiRs targeting either PRMT6 or LSD1 in mouse and human cells were designed with the online tool BLOCK-iT RNAi Designer: top and bottom oligonucleotides are synthesized, annealed and cloned into pcDNA6.2-GW/EmGFP-miR plasmid (SEQ ID NO: 231) using the BLOCK-iT Pol II miR RNAi kit (Invitrogen), to generate amiR sources to express the amiRs of interest in a cell receiving the plasmid; the plasmid comprises a spectinomycin resistance cassette.

Top and bottom oligonucleotides were generated according to Invitrogen instructions. Briefly, the top oligo sequences are generated by combining the following elements (from 5′ end to 3′ end):
1. 5′ TGCTG
2. Reverse complement of the 21-nucleotide sense target sequence of interest. This is the sequence encoding the mature miRNA.
3. Terminal loop sequence.
4. Nucleotides 1-8 (5′-3′) of sense target sequence.
5. Nucleotides 11-21 (5′-3′) of sense target sequence

To generate the bottom oligos sequence, the following steps are performed:
1. Remove 5′ TGCT from top oligo sequence (new sequence starts with G).
2. Take the reverse complement of the sequence from step 1.
3. Add CCTG to the 5′ end of the sequence from step 2.

Table 5 that follows provides the 5′-3′sequences of top and bottom oligonucleotides used to generate the amiR precursors of the examples, wherein, in the top oligonucleotides, the reverse complement of the 21-nucleotide sense target sequence of interest is underlined and the terminal loop coding sequence is in bold.

The amiRs' expression cassette then comprises (5′ to 3′) the following contiguous regions: a 5′ miR flanking region, the target-specific stem, terminal loop, stem-complementary region and 3′ miR flanking region.

This amiR cassette can be cloned within the 3′ untranslated region of any reporter gene end expressed therefrom under the control of an RNA polymerase type II promoter.

As a negative control, the control amiR sequence from pcDNA6.2-GW/EmGFP-miR-neg-control plasmid (provided with the kit) was used, containing a sequence that does not target any known vertebrate gene (control amiR: SEQ ID NO: 169: AAATGTACTGCGCGTGGAGAC). Top-10 competent E. coli were transformed, and positive clones were selected using EGFP forward primer 5′ GTCCTGCTGGAGTTCGTG-3′ (SEQ ID NO: 170).

amiR sequences against PRMT6 and LSD1 were sub-cloned downstream of EGFP under control of the CAG promoter in an adeno-associated virus (AAV) vector derived from pAAV-CAG-EGFP (Addgene #37825) as previously described¹⁵.

TABLE 5
Oligonucleotides (oligos) for generating amiRs
Target: mouse Prmt6

| amiR- | SEQ ID | Oligos sequences |
|---|---|---|
| Prmt6 | NO: | (T = top; B = bottom) |
| #1 | 139 | T: |
| | | TGCTGAGCACTCGTAGTACAGCTGGTGTTTTGGCCACTGACTGACACCAGCTGCTACGAGTGCT |
| | 140 | B: |
| | | CCTGAGCACTCGTAGCAGCTGGTGTCAGTCAGTGGCCAAAACACCAGCTGTACTACGAGTGCTC |
| #2 | 141 | T: |
| | | TGCTGCGGAGTAGCACTCGTAGTACAGTTTTGGCCACTGACTGACTGTACTACGTGCTACTCCG |
| | 142 | B: |
| | | CCTGCGGAGTAGCACGTAGTACAGTCAGTCAGTGGCCAAAACTGTACTACGAGTGCTACTCCGC |
| #3 | 143 | T: |
| | | TGCTGTTCAGGATGCCTAAGCGGTAGGTTTTGGCCACTGACTGACCTACCGCTGGCATCCTGAA |
| | 144 | B: |
| | | CCTGTTCAGGATGCCAGCGGTAGGTCAGTCAGTGGCCAAAACCTACCGCTTAGGCATCCTGAAC |
| #4 | 145 | T: |
| | | TGCTGTCTTCAGGATGCCTAAGCGGTGTTTTGGCCACTGACTGACACCGCTTACATCCTGAAGA |
| | 146 | B: |
| | | CCTGTCTTCAGGATGTAAGCGGTGTCAGTCAGTGGCCAAAACACCGCTTAGGCATCCTGAAGAC |
| #5 | 147 | T: |
| | | TGCTGTAGCGCAGAAGTATGCGCAGAGTTTTGGCCACTGACTGACTCTGCGCACTTCTGCGCTA |
| | 148 | B: |
| | | CCTGTAGCGCAGAAGTGCGCAGAGTCAGTCAGTGGCCAAAACTCTGCGCATACTTCTGCGCTAC |
| #6 | 149 | T: |
| | | TGCTGAATCAGACCATGTTGCCTTTCGTTTTGGCCACTGACTGACGAAAGGCAATGGTCTGATT |
| | 150 | B: |
| | | CCTGAATCAGACCATTGCCTTTCGTCAGTCAGTGGCCAAAACGAAAGGCAACATGGTCTGATTC |
| #7 | 151 | T: |
| | | TGCTGTATTAGATTAGAGTGCTCGCGGTTTTGGCCACTGACTGACCGCGAGCACTAATCTAATA |
| | 152 | B: |
| | | CCTGTATTAGATTAGTGCTCGCGGTCAGTCAGTGGCCAAAACCGCGAGCACTCTAATCTAATAC |

Target: mouse Lsd1

| amiR- | SEQ ID | Oligonucleotide sequences |
|---|---|---|
| Lsd1 | NO: | (T = top; B = bottom) |
| #1 | 153 | T: |
| | | TGCTGTTTCTCTGTATTCTACCTTGGGTTTTGGCCACTGACTGACCCAAGGTAATACAGAGAAA |
| | 154 | B: |
| | | CCTGTTTCTCTGTATTACCTTGGGTCAGTCAGTGGCCAAAACCCAAGGTAGAATACAGAGAAAC |
| #2 | 155 | T: |
| | | TGCTGTTTACTTGTTTGCTGACGACAGTTTTGGCCACTGACTGACTGTCGTCAAAACAAGTAAA |
| | 156 | B: |
| | | CCTGTTTACTTGTTTTGACGACAGTCAGTCAGTGGCCAAAACTGTCGTCAGCAAACAAGTAAAC |
| #3 | 234 | T: |
| | | TGCTGTTGATGAGAGGTATACATCACGTTTTGGCCACTGACTGACGTGATGTACCTCTCATCAA |
| | 235 | B: |
| | | CCTGTTGATGAGAGGTACATCACGTCAGTCAGTGGCCAAAACGTGATGTATACCTCTCATCAAC |

Target: human PRMT6

| amiR- | | |
|---|---|---|
| Prmt6 | | |
| (mature | | |
| miR | SEQ ID | Oligonucleotide sequences |
| SEQ ID) | NO: | (T = top; B = bottom) |
| #1 | 157 | T: |
| (Seq ID | | TGCTGAGCACGCTGTATCCGTTGTTGGTTTTGGCCACTGACTGACCAACAACGTACAGCGTGCT |
| No: | 158 | B: |
| 171) | | CCTGAGCACGCTGTACGTTGTTGGTCAGTCAGTGGCCAAAACCAACAACGGATACAGCGTGCTC |
| #2 | 159 | T: |
| (Seq ID | | TGCTGATAATAAGCACGCTGTATCCGGTTTTGGCCACTGACTGACCGGATACAGTGCTTATTAT |
| No: | 160 | B: |
| 172) | | CCTGATAATAAGCACTGTATCCGGTCAGTCAGTGGCCAAAACCGGATACAGCGTGCTTATTATC |
| #3 | 161 | T: |
| (Seq ID | | TGCTGTAAGGTGTGAGGTGTCAGGGAGTTTTGGCCACTGACTGACTCCCTGACCTCACACCTTA |
| No: | 162 | B: |
| 173) | | CCTGTAAGGTGTGAGGTCAGGGAGTCAGTCAGTGGCCAAAACTCCCTGACACCTCACACCTTAC |

Target: human LSD1

| amiR- | | |
|---|---|---|
| Lsd1 | | |
| (mature | | |
| miR | SEQ ID | Oligonucleotide sequences |
| SEQ ID) | NO: | (T = top; B = bottom) |
| #1 | 163 | T: |
| (Seq ID | | TGCTGATAACTGTGAACTCGGTGGACGTTTTGGCCACTGACTGACGTCCACCGTTCACAGTTAT |
| No: | 164 | B: |
| 174) | | CCTGATAACTGTGAACGGTGGACGTCAGTCAGTGGCCAAAACGTCCACCGAGTTCACAGTTATC |
| #2 | 165 | T: |
| (Seq ID | | TGCTGTGACGCTCTAAATAACTGTGAGTTTTGGCCACTGACTGACTCACAGTTTTAGAGCGTCA |
| No: | 166 | B: |
| 175) | | CCTGTGACGCTCTAAAACTGTGAGTCAGTCAGTGGCCAAAACTCACAGTTATTTAGAGCGTCAC |
| #3 | 167 | T: |
| (Seq ID | | TGCTGTTAGGAACCTTGACAGTGTCAGTTTTGGCCACTGACTGACTGACACTGAAGGTTCCTAA |
| No: | 168 | B: |
| 176) | | CCTGTTAGGAACCTTCAGTGTCAGTCAGTCAGTGGCCAAAACTGACACTGTCAAGGTTCCTAAC |

It is predicted that the oligonucleotides used to generate amiRs #1, #2 and #3 targeting human PRMT6, cloned in a vector and delivered in a cell, generate in that cell amiRs of sequence SEQ ID NO: 171, 172, 173 respectively and that the oligonucleotides used to generate amiRs #1, #2 and #3 targeting human LSD1, cloned in a vector and delivered in a cell, generate in that cell amiRs of sequence SEQ ID NO: 174, 175 and 176 respectively.

### Viral Production and Titration

AAV serotype 9 (AAV9) was produced using a slight modification of the adenovirus-free transient transfection method. Briefly, adherent HEK293 cells grown in roller bottles were transfected with three plasmids containing the adenovirus helper proteins, AAV Rep and Cap genes, and the ITR-flanked transgene expression cassette. Three days after transfection, cells were harvested, lysed by sonication, and treated with benzonase (Merck-Millipore). Vectors were purified using two successive ultracentrifugation rounds in caesium chloride density gradients. Full capsids were collected. The final product was formulated in sterile phosphate buffered saline containing 0.001% of pluronic F-68 (Sigma) and stored at −80° C. Titers of AAV vector samples were as follow: control amiR: 3.8*10¹² vg/mL; amiR-Prmt6: 4.18*10¹³ vg/mL; amiR-Lsd1: 9.8*10¹² vg/mL; amiR-Prmt6/Lsd1: 3.3*10¹³ vg/mL.

### Statistical Analysis

To compare measures across groups, student's two-sample t-tests and one-way analysis of variance (ANOVA) tests followed by Tukey's honest significant difference post-hoc tests were used for two and more than two group comparisons, respectively. To evaluate body weight and behavioural differences across genotype groups and treatments over time, two way-ANOVA with genotype, injection, and time as predictor variables followed by post hoc tests were used. For all tests, significance threshold was set at p≤0.05.

### Example 1

Expression of LSD1 and PRMT6 was verified in tissues of SBMA transgenic mice expressing human AR100Q mutation, knock-in SBMA mice in which AR exon 1 was replaced with the human AR exon 1 coding for a polyQ-expanded AR with 113Q, and available patient sample biopsies. Analysed tissues included skeletal muscle, liver, spinal cord, brainstem and heart. AR100Q male mice are non-symptomatic at 4 weeks of age (pre-symptomatic stage), start to show signs of muscle atrophy and motor dysfunction by 8 weeks of age (onset), and manifest signs of denervation by 12 weeks of age (late stage). Real-time PCR analysis showed that transcript levels of Lsd1 and Prmt6 were significantly increased 2-fold in skeletal muscle (quadriceps) and liver of male SBMA mice at the pre-symptomatic stage compared to wildtype (WT) male mice (FIG. 1a). In the brainstem, spinal cord, and heart, transcript levels of Lsd1 and Prmt6 were significantly higher only in the brainstem at 4 weeks of age and to a lower extent than skeletal muscle and liver. In muscle, upregulation was detected before the onset of motor dysfunction and denervation and was constant and persistent at all disease stages (4-12 weeks). Western blotting confirmed LSD1 and PRMT6 upregulation at the protein level in SBMA quadriceps muscle, but not spinal cord, compared to controls (FIG. 1b).

In muscle of female AR100Q mice, Lsd1 and Prmt6 transcript levels were upregulated only at late stage of disease and to a lower level (1.5-fold for Lsd1 and 1.8-fold for Prmt6) than male mice (FIG. 1c). Further, Lsd1 and Prmt6 were significantly upregulated 5-10-fold in the fast-twitch extensor digitorum longus (EDL) muscle of male AR100Q mice, and overexpression was normalized by surgical castration, consistent with the androgen-dependent nature of SBMA (FIG. 1d). In addition, Lsd1 and Prmt6 transcript levels were significantly upregulated 4-fold and 3-fold, respectively, in the skeletal muscle of AR113Q male knock-in mice compared to WT controls (FIG. 1e). Similar results were obtained in C2C12 myoblasts overexpressing AR100Q differentiated to myotubes, suggesting that this phenomenon occurs in muscle in a cell-autonomous fashion (FIG. 1f). Importantly, LSD1 and PRMT6 transcript levels also were upregulated 1.5-fold and 2.5-fold in the muscle of SBMA patients (FIG. 1g), thus underlying the relevance of these findings to disease.

### Example 2

To determine whether polyQ-expanded AR directly affects Lsd1 and Prmt6 transcription, putative androgen-responsive elements (AREs) were searched for in enhancers and distal/core promoters of Lsd1 and Prmt6. By bioinformatics analysis a putative ARE was found in the promoter of both genes (FIG. 1h). In a chromatin-immunoprecipitation (ChTP) assay in C2C12 myoblasts expressing AR with a normal (AR24Q) or pathogenic (AR100Q) polyQ tract, nonexpanded AR occupancy at the Lsd1 ARE was detected, which was enhanced by expanded polyQ. At the Prmt6 ARE, specific binding was found only for polyQ-expanded AR. These results provide a molecular mechanism underlying Lsd1 and Prmt6 overexpression in SBMA myofibers.

Taken together, these results indicate that Lsd1 and Prmt6 are overexpressed in a muscle cell-autonomous fashion, mainly as a result of an androgen-dependent AR toxic GOF.

### Example 3

The relationship of AR, PRMT6, and LSD1 was further investigated in the context of SBMA pathogenesis. To address whether LSD1 forms a complex with polyQ-expanded AR, Flag-tagged AR24Q or AR65Q were expressed with or without LSD1 in human embryonic kidney 293T (HEK293T) cells, and the cells were processed for co-immunoprecipitation assays. Importantly, overexpression of LSD1 per se did not modify AR expression under these experimental conditions (FIG. 11a). Pull-down of Flag-tagged normal and polyQ-expanded AR co-immunoprecipitated LSD1 (FIG. 11a). In addition to LSD1, at least three more protein isoforms derived by Lsd1 alternative splicing (LSD1-2a, LSD-8a, and LSD1-2a/8a) formed a complex with normal and polyQ-expanded AR both in the absence and presence of DHT (FIG. 11b).

Immunofluorescence analysis showed that normal and polyQ-expanded AR localize with endogenous LSD1 in motor neuron-derived MN1 cells treated with vehicle or DHT, in patient-derived IPSCs differentiated to motor neurons, and in the spinal cord of an SBMA patient. Notably, AR, LSD1, and PRMT6 were all present in the nuclei of human IPSC-derived motor neurons. To assess protein-protein interactions in the subcellular compartments of intact cells, a proximity ligation assay (PLA) was used, which is based on oligonucleotide-conjugated secondary antibodies detecting in situ protein-protein interactions (FIG. 2a). The interaction of polyQ-expanded AR and PRMT6 was also assessed, which is enhanced in the presence of the ligand dihydrotestosterone (DHT). AR24Q and AR100Q formed a complex with endogenous LSD1 in a DHT-independent manner.

Although LSD1 is a co-activator of normal AR, whether it also works as a co-activator of polyQ-expanded AR is unclear. To address this question, AR activity was assessed by expressing AR24Q and AR65Q driven by the cytomegalovirus promoter and measuring the activity of a luciferase reporter under the control of an androgen-responsive element. Both GOF (overexpression) and LOF (knock-down) approaches were applied. In HEK293T cells, LSD1 overexpression enhanced AR24Q and AR65Q transactivation by 1.4-fold in DHT-treated cells, and it had no effect in vehicle-treated cells, indicating that LSD1 works as a transcriptional co-activator for AR (FIG. 2b). Similar results were obtained by expressing AR12Q and AR55Q under the control of the eukaryotic promoter, elongation factor 1α (FIG. 12a). Further, LSD1-2a, LSD1-8a, and LSD1-2a/8a transactivated normal and polyQ-expanded AR to a similar extent as LSD1 in HEK293T cells (FIG. 12b) and MN1 cells (FIG. 2c, FIG. 12c).

### Example 4

Endogenous LSD1 was suppressed by CRISPR/Cas9 technology. Using Cas9 with different single gRNAs in HEK293T cells, partial knock-down of LSD1 was obtained, while concomitant use of two gRNAs produced a large in-frame deletion of LSD1 exons encoding essential catalytic regions, thus producing an enzymatically dead LSD1 fragment (FIG. 2d, FIG. 12d). Partial (50%-70%) and complete knock-out of LSD1 reduced AR24Q and AR65Q transactivation by 40%-50% and 80%, respectively, indicating a dose-dependent effect of endogenous LSD1 on androgen-induced AR transactivation (FIG. 2d). Consistently, polyQ-expanded AR transactivation also was reduced upon knock-down of LSD1. In addition, CRISPR-Cas9 knock-down of Lsd1 significantly diminished AR24Q and AR100Q transactivation in MN1 cells (FIG. 2e). Collectively, these results show that LSD1 forms a complex with and works as a co-activator of polyQ-expanded AR, and that CRISPR-Cas9 knock-down of Lsd1 is capable of diminishing transactivation of polyQ-expanded AR.

### Example 5

Both LSD1 and PRMT6 have an LXXLL motif (FIG. 3a), which mediates the interaction of transcription co-factors with steroid receptors through the activating function-2 (AF-2) surface in the ligand-binding domain. Consistently, LSD1 failed to transactivate AR bearing the E897K (where E is glutamic acid and K is lysine) mutation that disrupts co-factor recruitment through AF-2 (FIG. 3b). An LSD1 with a defective LXXLL motif (mutated to LXXAA, where A is alanine) also failed to transactivate AR (FIG. 3c). The catalytic-inactive LSD1 mutant K685A failed to transactivate normal and polyQ-expanded AR (FIG. 3d).

Further, treatment of mock-transfected cells with the LSD1 catalytic inhibitor tranylcypromine (TCP) reduced polyQ-expanded AR transactivation induced by DHT (FIG. 3e); treatment of cells with the selective LSD1 inhibitor SP-2509, which inhibits the association of LSD1 with CoREST but does not affect LSD1 enzymatic activity, did not modify polyQ-expanded AR transactivation, further supporting the relevance of LSD1 catalytic activity for AR transactivation. Notably, it was found decreased histone 3 lysine 4 methylation (H3K4me2) in C2C12 myotubes expressing AR100Q, thus confirming that LSD1 is overactivated in SBMA myotubes (FIG. 3f). Taken together, these observations indicate that LSD1 requires its catalytic activity and the AR AF-2 surface to transactivate both normal and polyQ-expanded AR through a mechanism that involves histone modification.

### Example 6

PRMT6 and LSD1 are here shown to be both co-activators of AR and to be overexpressed in SBMA skeletal muscle. It was then tested whether they synergistically contribute to the toxic GOF of polyQ-expanded AR. First it was addressed whether LSD1 and PRMT6 interact with each other in HEK293T cells, after verifying that neither overexpression nor silencing of PRMT6 modified expression of endogenous and exogenously expressed LSD1 and vice versa (FIG. 13a). PRMT6 immunoprecipitation pulled down LSD1, indicating that PRMT6 and LSD1 interact in HEK293T cells. Further, PLA in MN1 cells expressing AR24Q and AR100Q showed that endogenous PRMT6 interacted with endogenous LSD1, and this interaction was not modified by DHT (FIG. 4a). LSD1 and PRMT6 then interact with each other and with AR through the AF-2 surface. MN1 Lsd1 CRISPR-Cas9 knock-down cells were transduced with lentivirus expressing scramble shRNA or two shRNAs against Prmt6: shPRMT6 #1 (SEQ ID NO:232) and shPRMT6 #2 (SEQ ID NO:233) to silence endogenous Prmt6.

By Western blotting it was verified that knock-down of either Lsd1 or Prmt6 did not modify PRMT6, and LSD1 levels, respectively nor AR levels (FIG. 4b). Next protein-protein interactions was assessed by PLA in MN1 cells. Strikingly, the interaction of normal and polyQ-expanded AR with LSD1 was significantly diminished upon Prmt6 silencing, as was the interaction of AR with PRMT6 upon Lsd1 silencing, even if this interaction is aberrantly enhanced by expanded polyQ (FIG. 4a). In an immunoprecipitation assay, it was confirmed AR/LSD1/PRMT6 interaction in the quadriceps muscle of WT and knock-in SBMA mice expressing AR113Q (FIG. 4c).

Next, it was assessed whether LSD1 and PRMT6 cooperatively transactivate AR. Overexpression of LSD1 alone, PRMT6 alone, and both coactivators together, enhanced transactivation of normal AR by 1.3-, 3.1-, and 4.5-fold, respectively, and that of polyQ-expanded AR by 1.5-, 3.4-, and 6.6-fold (FIG. 4d). Notably, polyQ-expanded AR transactivation by LSD1/PRMT6 was increased compared to normal AR. AR transactivation by PRMT6 is negatively regulated by phosphorylation at AKT consensus sites, ²¹⁰RXRXXS²¹⁵ and ⁷⁸⁷RXRXXS⁷⁹² (where R is arginine and S is serine). Interestingly, AR transactivation by LSD1 and PRMT6 was significantly enhanced by phospho-defective substitutions with alanine (S215A, S792A) compared to AR65Q with intact S215 and S792 residues, suggesting that AR transactivation by LSD1 and PRMT6 is negatively modulated by phosphorylation at AKT consensus sites (FIG. 13b).

### Example 7

A pharmacological and genetic LOF approach was took to assess the effect of inhibition of endogenous (mock-transfected cells) and overexpressed LSD1 and PRMT6 on AR activity via transcriptional assays. TCP and the PRMT inhibitor adenosine dialdehyde (AdOx) reduced normal and polyQ-expanded AR transactivation induced by DHT (FIG. 4d), indicating that AR requires both LSD1 and PRMT6 function for full transactivation. Notably, the effect of overexpression of LSD1 on AR transactivation was reduced not only by TCP as expected but also by AdOx. Similarly, the effect of overexpression of PRMT6 was attenuated by AdOx as well as TCP. Combined treatment with TCP/AdOx further attenuated AR transactivation both in mock-transfected cells and in cells overexpressing LSD1, PRMT6, and LSD1/PRMT6. However, AdOx is a pan-PRMT inhibitor, and TCP can have additional effects on enzymes other than LSD1.

### Example 8

Endogenous LSD1 and PRMT6 were genetically silenced by CRISPR/Cas9 technology (FIG. 2d, FIG. 4e).

Knock-down of LSD1 and PRMT6 with two independent guide RNAs alone and together attenuated normal and polyQ-expanded AR transactivation induced by DHT (FIG. 4e).

Further, PRMT6-induced transactivation of normal and polyQ-expanded AR was significantly reduced by genetic silencing of endogenous LSD1 (FIG. 13c).

### Example 9

To determine whether LSD1 modifies the SBMA phenotype and synergistically cooperates with PRMT6 to enhance toxicity in vivo, SBMA flies were crossed with flies expressing RNA interference (RNAi) targeting the Drosophila ortholog of LSD1, dLsd1, and the Drosophila ortholog of PRMT6, Dart8, obtained from the Vienna Drosophila Resource Center (VDRC, stock ID for DART8: v100228, and dLsd1: v106147). As control, flies expressing an AR without the polyQ tract (AR0Q) were used. AR0Q flies fed DHT did not show any phenotype (FIG. 5a). To model SBMA, flies expressing an AR with 52Q (AR52Q) were used. AR52Q flies developed degeneration of the posterior side of the eye. Silencing of dLsd1 by about 40% did not modify polyQ-expanded AR toxicity, whereas silencing of Dart8 by about 50% had a significant yet modest effect on polyQ-expanded AR toxicity (FIG. 5a-c). Consistent with the synergistic effect on polyQ-expanded AR transactivation, silencing of both dLsd1 and Dart8 together strongly suppressed the DHT-induced degenerative phenotype caused by polyQ-expanded AR. This evidence supports the synergistic cooperation of LSD1 and PRMT6 to enhance polyQ-expanded AR toxic GOF in vivo and that silencing of both the coactivators synergistically reduces the toxic GOF of mutated AR.

### Example 10

The results described above support the development of a therapeutic strategy targeting AR co-regulators, according to the present invention. 83 and 51 artificial miRNAs (amiRs) were designed to silence mouse Lsd1 and Prmt6, respectively. Using the best alignments targeting all known mouse Lsd1 isoforms and Prmt6, top-ranking amiRs were selected for in vitro validation (FIG. 6a, Table 5). In MN1 cells, amiR-Lsd1 #1 silenced Lsd1 expression by 50%, whereas amiR-Lsd1 #2 and #3 silenced expression by 70%. Because Lsd1 knock-out is embryonic lethal, whereas its haploinsufficiency is not associated with major consequences in mouse, amiR-Lsd1 #1 was selected for further analysis. Prmt6 amiRs #2, #6, and #7 significantly silenced Prmt6 to a similar extent (~50%); amiR-Prmt6 #6 was pursued for further analysis.

MN1 cells expressing AR100Q, which showed reduced cell viability compared to MN1 cells expressing AR24Q in the presence of DHT, had significantly increased cell viability upon simultaneous silencing of Lsd1 and Prmt6 (FIG. 6b), consistent with results from SBMA flies.

To move in vivo, an adeno-associated virus subtype 9 (AAV9) that expresses green fluorescent protein (GFP) was used as amiRs source (FIG. 6c). To set up the conditions of viral infection, a single intraperitoneal injection of amiR-Lsd1/Prmt6 was performed at concentrations of 2¹⁰ and 8¹⁰ virion particles at 3 weeks of age. By analysing the biodistribution of AAV9 expressing control amiR, viral particles were detected in several tissues including liver, heart, quadriceps, and spinal cord (FIG. 6d). Western blotting detected GFP expression in several tissues including skeletal muscle (FIG. 6e). Transduction of amiR-Lsd1/Prmt6 in AR100Q mice significantly reduced Lsd1 and Prmt6 transcript levels, with no effect on mouse and human AR transcript levels, in the skeletal muscle (FIG. 6f). Western blotting confirmed reduced expression of LSD1 and PRMT6 proteins in skeletal muscle, but not spinal cord, liver, white adipose tissue, and lungs, except LSD1 that was significantly reduced in the heart (FIG. 6g and FIG. 14).

As LSD1 and PRMT6 are epigenetic writers that modify gene expression and AR is a transcription factor active in several tissues, the transcript levels of AR, LSD1, and PRMT6 target genes were measured in tissues other than skeletal muscle. Treatment did not modify the expression of selected target genes in most tissues, thus excluding a general effect of gene transcription (FIG. 15). These observations show that AAV9-mediated delivery of amiR-Lsd1/Prmt6 is an effective strategy to silence AR co-regulators in vivo with no effect on AR gene expression.

### Example 11

Efficacy of amiRs of Example 10 was Tested in AR100Q Mice.

First, transcriptomic analysis by RNA-seq was performed in quadriceps muscle of 13-week-old control and amiR-Lsd1/Prmt6-treated AR100Q mice and WT mice. 6,583 differentially expressed genes (DEGs) (4,158 upregulated and 2,425 downregulated genes, GSE193539) were identified in untreated SBMA muscles compared to WT muscles (absolute log 2 fold change >2, corrected p≤0.01) (FIG. 7a), consistent with recent observations of a high number of altered genes in the tibialis anterior muscle at 11 weeks in the same SBMA mouse model. The effect of treatment was analysed in WT and AR100Q mice, 1,129 DEGs (488 upregulated and 641 downregulated genes) were found specifically in AR100Q mice. Of note, 285 genes were completely rescued, showing transcriptional levels comparable to WT mice and 389 genes demonstrated partial rescue (FIG. 7b).

Also, by performing functional enrichment analysis on the rescued genes, statistical significance was obtained for several terms including “skeletal muscle contraction”, “muscle structure development”, “myofibril assembly”, “sarcoplasmic reticulum calcium ion transport”, “collagen chain trimerization”, “myofibril assembly and metabolism”, “fructose glycogen metabolism”, “protein nitrosylation”, “generation of precursor metabolites and energy”, and “purine nucleoside monophosphate metabolic process” (FIG. 7c,d). Interestingly, most (~80%) of GO biological processes previously identified as enriched in muscles of female mice expressing polyQ-expanded AR and treated with androgens were found to have an overlap coefficient of >0.5 with the GO biological processes enriched by the rescued genes (65% with overlap coefficient of 1). This suggests that genes in these categories are transcriptionally dysregulated by mutant AR and partially or totally normalized upon Lsd1 and Prmt6 silencing.

Comparing the data with published transcriptomic analyses on SBMA muscles expressing either AR113Q (knock-in model), AR97Q (another transgenic model), or WT AR only in the muscle (HSA-AR)¹¹, it was noted that fifty-two of 153 (34%) genes dysregulated in the HSA-AR model were rescued by Lsd1 and Prmt6 silencing. Similarly, 58 of 204 (28%) and 31 of 159 (20%) genes dysregulated in the AR97Q and AR113 models were rescued, respectively. Moreover, only 17% of the genes dysregulated in a LOF model of AR¹² were rescued by amiR treatment, showing a limited reduction of AR physiological functions. Overall, the data demonstrate an effect of the genetic silencing, according to preferred embodiments of the invention, on polyQ-expanded AR toxic GOF.

### Example 12

Next, a preclinical study was performed to assess the effect of Lsd1 Prmt6 silencing on the mouse disease phenotype. A randomized cohort of male transgenic AR100Q mice and WT siblings was assigned to either control amiR or amiR-Lsd1/Prmt6. It was first verified that the genetic silencing therapeutic strategy did not modify body weight and motor function of WT mice, thus excluding undesired effects from genetic manipulation of target genes (FIG. 8a). This is particularly important for Lsd1, for which deletion in mouse is embryonic lethal.

Then the effect of treatment was analysed on the SBMA phenotype. AR100Q mice show reduced body weight and motor dysfunction starting around 8 weeks of age. Treatment with amiR-Lsd1/Prmt6 significantly increased their body weight at 9-11 weeks of age and improved muscle strength by hanging wire and motor coordination by rotarod tasks (FIG. 8a).

SBMA muscle is characterized by a glycolytic-to-oxidative fibre-type switch. Consistent with the role of LSD1 on muscle metabolism, amiR-Lsd1/Prmt6 reduced (p=0.05) the number of oxidative fibres from 49% in vehicle-treated mice to 37% in amiR-Lsd1/Prmt6-treated mice (FIG. 8b). A key aspect of skeletal muscle pathology in SBMA is functional denervation associated with the upregulation of genes, such as muscle associated receptor tyrosine kinase (Musk), myogenin (MyoG), and neural cell adhesion molecule (NCAM), which are induced upon dysfunctional communication between the motor neuron and the innervated myofiber⁸. Musk, MyoG, and NCAM were upregulated in the muscle of AR100Q mice and were significantly reduced by treatment (FIG. 8c). It is noteworthy that these genes are regulated by LSD1. AR100Q forms 2% SDS-resistant aggregates in muscle, which can be detected as high molecular weight (HMW) species that accumulate in the stacking portion of a polyacrylamide gel 8. Consistent with findings that PRMT6 enhances polyQ-expanded AR aggregation, Western blotting showed that silencing Lsd1 and Prmt6 reduced accumulation of HMW species in muscle, while increasing the amount of monomeric AR (FIG. 8d). Together, these results show that silencing Lsd1 and Prmt6 attenuates the phenotype of a severe murine model of SBMA.

### Example 13

The genetic silencing strategy was validated also in human cells, 3 amiRs were designed to silence human LSD1 and PRMT6 (Table 5). Target silencing was verified in HEK293T and in the androgen-sensitive prostate cancer cell line, LNCaP, because LSD1 and PRMT6 are overexpressed in prostate cancer and correlate with cancer aggressiveness (FIG. 9a)^{4,7}. In HEK293T cells, silencing of both LSD1 and PRMT6 modified the expression of two genes regulated by AR and its co-regulators, namely ATP2A2, coding for SERCA2, and CDKN1A, coding for p21^{CIP1} (FIG. 9b). In LNCaP cells, a significant reduction in proliferation in the cells targeting either LSD1 or PRMT6 was observed, thus showing that these amiRs exert a biological effect in prostate cancer cells (FIG. 9c). These observations validate amiR-mediated silencing of two fundamental AR co-regulators in human cells, suggesting a potential translation of this strategy to patients suffering from SBMA and possibly other AR-associated GOF diseases, namely prostate cancer and those types of cancer with a sex-bias.

### Example 14

Further preclinical studies were performed to assess the effect of Lsd1 pharmacological inhibition on the mouse disease phenotype. A randomized cohort of male transgenic AR100Q mice and WT siblings was assigned to either control or treated group.

Two safe MAO-B inhibitor, used in clinics as antidepressant, were tested in the amelioration of the SBMA-like phenotype of AR100Q mice: phenelzine and tranylcypromine (TCP).

These two drugs link covalently to FAD, the LSD1's cofactor, resulting in an irreversible blockade of LSD1. Treatments were started at week 4 (pre-symptomatic stage, but when LSD1 and PRMT6 upregulation is already detected), until death.

Phenelzine was administered at a dose of 30 mg/Kg in drinking water; three times a week; TCP was administered at a dose of 6 mg/Kg intraperitoneal, three times a week.

Mice were randomized and the operators were blind for genotype and treatment. The phenotype was analyzed by looking at muscle strength with grip strength test.

Since 10 weeks of age, AR100Q mice (Tg) showed a significant decreased strength compared to wild-type mice (WT). The phenotype of AR100Q mice was ameliorated by both Phenelzine and TCP (FIG. 16 a and b, respectively). LSD1 inhibition with small molecules thus showed a beneficial effect, supporting the efficacy of small molecule inhibitors of AR coactivators, either alone or in combination with genetic silencers, to treat diseases associated with gain-of-function of AR and/or with overexpression of an AR coactivator.

Sequences

SEQ ID NO: 1
>NM_018137.3
Homo sapiens protein arginine methyltransferase 6 (PRMT6), mRNA
gtgccgcgctacgcccgccgggagccgggcagagcggccaagatgtcgcagcccaagaaaagaaagcttgagt
cggggggcggcggcgaaggaggggagggaactgaagaggaagatggcgcggagcgggaggcggccctggagcg
accccggaggactaagcgggaacgggaccagctgtactacgagtgctactcggacgtttcggtccacgaggag
atgatcgcggaccgcgtccgcaccgatgcctaccgcctgggtatccttcggaactgggcagcactgcgaggca
agacggtactggacgtgggcgcgggcaccggcattctgagcatcttctgtgcccaggccggggcccggcgcgt
gtacgcggtagaggccagcgccatctggcaacaggcccgggaggtggtgcggttcaacgggctggaggaccgg
gtgcacgtcctgccgggaccagtggagactgtagagttgccggaacaggtggatgccatcgtgagcgagtgga
tgggctacggactcctgcacgagtccatgctgagctccgtcctccacgcgcgaaccaagtggctgaaggaggg
cggtcttctcctgccggcctccgccgagctcttcatagcccccatcagcgaccagatgctggaatggcgcctg
ggcttctggagccaggtgaagcagcactatggtgtggacatgagctgcctggagggcttcgccacgcgctgtc
tcatgggccactcggagatcgttgtgcagggattgtccggcgaggacgtgctggcccggccgcagcgctttgc
tcagctagagctctcccgcgccggcttggagcaggagctggaggccggagtgggcgggcgcttccgctgcagc
tgctatggctcggcgcccatgcatggctttgccatctggttccaggtgaccttccctggaggggagtcggaga
aacccctggtgctgtccacctcgccttttcacccggccactcactggaaacaggcgctcctctacctgaacga
gccggtgcaagtggagcaagacacggacgtttcaggagagatcacgctgctgccctcccgggacaacccccgt
cgcctgcgcgtgctgctgcgctacaaagtgggagaccaggaggagaagaccaaagactttgccatggaggact
gagcgttgccttttctcccagctacctcccaaagcagcctgacctgcgtgggagaggcgtagcgaggtcggag
gggaaagggagatcccacgtgcaagtagggggaatatctcccccttttccctcatagcctctagggagggaga
gtgacttcattctccatttgaagagattcttctggtgatgtttacttaaaaagtgatccccctcaacaacgga
tacagcgtgcttattattgggcatttagcctcaaaagcatgtagtaccaagcacttgtatttccgtatatttt
gtttcgcgggggagtgagggggaagaacacggatgaaaatgtcagtttttgaagggtccatgcacatccctga
cacctcacaccttatctaagtctgaagctggggagaaaggggttcatttagacttcatacatttccagtacga
ctttagtatctctccagagccatattttctcagtccgaattaattccccctccctaggtgcctgtaggctatg
gtacttcttcctcattgttttctaggtaaacttcactactggtaattaaggggaaggatatgaggaagcagtt
taaatagccctgttctcattactctgaccacatacatcatagggtgctaaagttgatgaacacattaatccgt
taagtaaaatggactttgtaattgtacagcatacctaagaaactcagaaggtgcatttaagagagagacctga
aagaaatagtatggatttttaaaaattcttgtctctactattataaccaaaaaatatttcttgtatgtcccat
aaaaatatttgtgtaattcttatgaaacaggctggtagaggaggtttctgagcctagcccaagggcttattca
tcaccatgggtaaattatttaaactcacttaattaaggaaaatattttcccagctagaaaagtatactcattc
tcatttaaactctctcatttggagggatcatgtgagttggcctacttacaagtagtgaaagttcccttttcag
ttttgttttgttttgttttgtttttctctttcactcagccaaatgtgaaagttgtgaatttaggaaaatcact
tgtaatgaagtgtgaatcttgttatcaaatttatttctctgatgtttccttccttatccttgtagccaataaa
acattgacattctcacgttttatagatgaggtaaaaagtcttgtgtgctgtgagttataatgcttttgccttt
ttaatattattagttcttaagtgttacagccccttcagaatataacttcaggacaattcaaactatgcttaat
gtatgattttcgagcttctgtatgctaagaaaataggtgtgaaaaactggtgttctgaaatagcctaacattt
attgtaattctgaattttctgcccttttattcattgcatattaaagtattagagtataaaaactaa
SEQ ID NO: 2
>NM_001009999.3
Homo sapiens lysine demethylase 1A (KDM1A), transcript variant 1, mRNA
ggcgcgtgcgtacgcgacggcggttggcggcgcgcgggcagcgtgaagcgaggcgaggcaaggcttttcggac
ccacggagcgacagagcgagcggcccctacggccgtcggcggcccggcggcccgagatgttatctgggaagaa
ggcggcagccgcggcggcggcggctgcagcggcagcaaccgggacggaggctggccctgggacagcaggcggc
tccgagaacgggtctgaggtggccgcgcagcccgcgggcctgtcgggcccagccgaggtcgggccgggggcgg
tgggggagcgcacaccccgcaagaaagagcctccgcgggcctcgccccccgggggcctggcggaaccgccggg
gtccgcagggcctcaggccggccctactgtcgtgcctgggtctgcgacccccatggaaactggaatagcagag
actccggaggggcgtcggaccagccggcgcaagcgggcgaaggtagagtacagagagatggatgaaagcttgg
ccaacctctcagaagatgagtattattcagaagaagagagaaatgccaaagcagagaaggaaaagaagcttcc
cccaccaccccctcaagccccacctgaggaagaaaatgaaagtgagcctgaagaaccatcggggcaagcagga
ggacttcaagacgacagttctggagggtatggagacggccaagcatcaggtgtggagggcgcagctttccaga
gccgacttcctcatgaccggatgacttctcaagaagcagcctgttttccagatattatcagtggaccacaaca
gacccagaaggtttttcttttcattagaaaccgcacactgcagttgtggttggataatccaaagattcagctg
acatttgaggctactctccaacaattagaagcaccttataacagtgatactgtgcttgtccaccgagttcaca
gttatttagagcgtcatggtcttatcaacttcggcatctataagaggataaaacccctaccaactaaaaagac
aggaaaggtaattattataggctctggggtctcaggcttggcagcagctcgacagttacaaagttttggaatg
gatgtcacacttttggaagccagggatcgtgtgggtggacgagttgccacatttcgcaaaggaaactatgtag
ctgatcttggagccatggtggtaacaggtcttggagggaatcctatggctgtggtcagcaaacaagtaaatat
ggaactggccaagatcaagcaaaaatgcccactttatgaagccaacggacaagctgacactgtcaaggttcct
aaagagaaagatgaaatggtagagcaagagtttaaccggttgctagaagctacatcttaccttagtcatcaac
tagacttcaatgtcctcaataataagcctgtgtcccttggccaggcattggaagttgtcattcagttacaaga
gaagcatgtcaaagatgagcagattgaacattggaagaagatagtgaaaactcaggaagaattgaaagaactt
cttaataagatggtaaatttgaaagagaaaattaaagaactccatcagcaatacaaagaagcatctgaagtaa
agccacccagagatattactgccgagttcttagtgaaaagcaaacacagggatctgaccgccctatgcaagga
atatgatgaattagctgaaacacaaggaaagctagaagaaaaacttcaggagttggaagcgaatcccccaagt
gatgtatatctctcatcaagagacagacaaatacttgattggcattttgcaaatcttgaatttgctaatgcca
cacctctctcaactctctcccttaagcactgggatcaggatgatgactttgagttcactggcagccacctgac
agtaaggaatggctactcgtgtgtgcctgtggctttagcagaaggcctagacattaaactgaatacagcagtg
cgacaggttcgctacacggcttcaggatgtgaagtgatagctgtgaatacccgctccacgagtcaaaccttta
tttataaatgcgacgcagttctctgtacccttcccctgggtgtgctgaagcagcagccaccagccgttcagtt
tgtgccacctctccctgagtggaaaacatctgcagtccaaaggatgggatttggcaaccttaacaaggtggtg
ttgtgttttgatcgggtgttctgggatccaagtgtcaatttgttcgggcatgttggcagtacgactgccagca
ggggtgagctcttcctcttctggaacctctataaagctccaatactgttggcactagtggcaggagaagctgc
tggtatcatggaaaacataagtgacgatgtgattgttggccgatgcctggccattctcaaagggatttttggt
agcagtgcagtacctcagcccaaagaaactgtggtgtctcgttggcgtgctgatccctgggctcggggctctt
attcctatgttgctgcaggatcatctggaaatgactatgatttaatggctcagccaatcactcctggcccctc
gattccaggtgccccacagccgattccacgactcttctttgcgggagaacatacgatccgtaactacccagcc
acagtgcatggtgctctgctgagtgggctgcgagaagcgggaagaattgcagaccagtttttgggggccatgt
atacgctgcctcgccaggccacaccaggtgttcctgcacagcagtccccaagcatgtgagacagatgcattct
aagggaagaggcccatgtgcctgtttctgccatgtaaggaaggctcttctagcaatactagatcccactgaga
aaatccaccctggcatctgggctcctgatcagctgatggagctcctgatttgacaaaggagcttgcctccttt
gaatgacctagagcacagggaggaacttgtccattagtttggaattgtgttcttcgtaaagactgaggcaagc
aagtgctgtgaaataacatcatcttagtcccttggtgtgtggggtttttgttttttttttatattttgagaat
aaaacttcatataaaattg

SEQ ID NO: 3-53: amiRs' human PRMT6 TARGET
SEQUENCES (cDNA)

| SEQ ID | TARGET SEQUENCE |
|---|---|
| NO: | (5′-3′ cDNA) |
| 3 | gggaactgaagaggaagatgg |
| 4 | aacaggtggatgccatcgtga |
| 5 | tcagcgaccagatgctggaat |
| 6 | caggtgaagcagcactatggt |
| 7 | gtgaagcagcactatggtgtg |
| 8 | actatggtgtggacatgagct |
| 9 | ggagatcgttgtgcagggatt |
| 10 | agatcgttgtgcagggattgt |
| 11 | ccatgcatggctttgccatct |
| 12 | atgcatggctttgccatctgg |
| 13 | tgcatggctttgccatctggt |
| 14 | gcatggctttgccatctggtt |
| 15 | ttgccatctggttccaggtga |
| 16 | tggagcaagacacggacgttt |
| 17 | cacggacgtttcaggagagat |
| 18 | caacaacggatacagcgtgct |
| 19 | caacggatacagcgtgcttat |
| 20 | aacggatacagcgtgcttatt |
| 21 | cggatacagcgtgcttattat |
| 22 | ggatacagcgtgcttattatt |
| 23 | cagcgtgcttattattgggca |
| 24 | cgtgcttattattgggcattt |
| 25 | gcttattattgggcatttagc |
| 26 | attattgggcatttagcctca |
| 27 | gtagtaccaagcacttgtatt |
| 28 | atccctgacacctcacacctt |
| 29 | tccctgacacctcacacctta |
| 30 | ccctgacacctcacaccttat |
| 31 | tgacacctcacaccttatcta |
| 32 | acacctcacaccttatctaag |
| 33 | cacaccttatctaagtctgaa |
| 34 | acaccttatctaagtctgaag |
| 35 | tcatacatttccagtacgact |
| 36 | catacatttccagtacgactt |
| 37 | tccagtacgactttagtatct |
| 38 | tacgactttagtatctctcca |
| 39 | aggtgcctgtaggctatggta |
| 40 | cctgtaggctatggtacttct |
| 41 | ggctatggtacttcttcctca |
| 42 | gttctcattactctgaccaca |
| 43 | ttctcattactctgaccacat |
| 44 | actctgaccacatacatcata |
| 45 | acatacatcatagggtgctaa |
| 46 | catacatcatagggtgctaaa |
| 47 | atacatcatagggtgctaaag |
| 48 | acatcatagggtgctaaagtt |
| 49 | catcatagggtgctaaagttg |
| 50 | tcatagggtgctaaagttgat |
| 51 | gggtgctaaagttgatgaaca |
| 52 | gcatacctaagaaactcagaa |
| 53 | cagccaaatgtgaaagttgtg |

SEQ ID NO: 54-138: amiRs' human LSD1 TARGET
SEQUENCES (DNA)

| SEQ ID | TARGET SEQUENCE |
|---|---|
| NO: | (5′-3′ cDNA) |
| 54 | ggaaactggaatagcagagac |
| 55 | gaaactggaatagcagagact |
| 56 | gacgacagttctggagggtat |
| 57 | acgacagttctggagggtatg |
| 58 | acagttctggagggtatggag |
| 59 | tttccagagccgacttcctca |
| 60 | ttcctcatgaccggatgactt |
| 61 | catgaccggatgacttctcaa |
| 62 | gaccggatgacttctcaagaa |
| 63 | acactgcagttgtggttggat |
| 64 | cactgcagttgtggttggata |
| 65 | ctgcagttgtggttggataat |
| 66 | cagttgtggttggataatcca |
| 67 | tgtccaccgagttcacagtta |
| 68 | gtccaccgagttcacagttat |
| 69 | caccgagttcacagttattta |
| 70 | ccgagttcacagttatttaga |
| 71 | cgagttcacagttatttagag |
| 72 | tcacagttatttagagcgtca |
| 73 | cacagttatttagagcgtcat |
| 74 | agttatttagagcgtcatggt |
| 75 | ttatttagagcgtcatggtct |
| 76 | tatttagagcgtcatggtctt |
| 77 | ggtggacgagttgccacattt |
| 78 | gacgagttgccacatttcgca |
| 79 | ttgccacatttcgcaaaggaa |
| 80 | tgccacatttcgcaaaggaaa |
| 81 | catttcgcaaaggaaactatg |
| 82 | gccatggtggtaacaggtctt |
| 83 | ggtaacaggtcttggagggaa |
| 84 | aggtcttggagggaatcctat |
| 85 | ggaatcctatggctgtggtca |
| 86 | atcctatggctgtggtcagca |
| 87 | caagctgacactgtcaaggtt |
| 88 | aagctgacactgtcaaggttc |
| 89 | ctgacactgtcaaggttccta |
| 90 | tgacactgtcaaggttcctaa |
| 91 | gacactgtcaaggttcctaaa |
| 92 | actgtcaaggttcctaaagag |
| 93 | ctgtcaaggttcctaaagaga |
| 94 | tgtcaaggttcctaaagagaa |
| 95 | gtcaaggttcctaaagagaaa |
| 96 | agagcaagagtttaaccggtt |
| 97 | gagcaagagtttaaccggttg |
| 98 | aagagtttaaccggttgctag |
| 99 | ctccatcagcaatacaaagaa |
| 100 | cagcaatacaaagaagcatct |
| 101 | ctgaagtaaagccacccagag |
| 102 | taaagccacccagagatatta |
| 103 | ccagagatattactgccgagt |
| 104 | cagagatattactgccgagtt |
| 105 | gagatattactgccgagttct |
| 106 | agatattactgccgagttctt |
| 107 | gatattactgccgagttctta |
| 108 | tgaccgccctatgcaaggaat |
| 109 | gccctatgcaaggaatatgat |
| 110 | cctatgcaaggaatatgatga |
| 111 | cagacaaatacttgattggca |
| 112 | ctctctcaactctctccctta |
| 113 | tctctcaactctctcccttaa |
| 114 | taagcactgggatcaggatga |
| 115 | tgggatcaggatgatgacttt |
| 116 | tcaggatgatgactttgagtt |
| 117 | aggatgatgactttgagttca |
| 118 | atgactttgagttcactggca |
| 119 | gtggctttagcagaaggccta |
| 120 | tttagcagaaggcctagacat |
| 121 | tagcagaaggcctagacatta |
| 122 | aaggcctagacattaaactga |
| 123 | aggcctagacattaaactgaa |
| 124 | agcccaaagaaactgtggtgt |
| 125 | cccaaagaaactgtggtgtct |
| 126 | aagaaactgtggtgtctcgtt |
| 127 | agaaactgtggtgtctcgttg |
| 128 | agccgattccacgactcttct |
| 129 | gccgattccacgactcttctt |
| 130 | cgattccacgactcttctttg |
| 131 | cgactcttctttgcgggagaa |
| 132 | actcttctttgcgggagaaca |
| 133 | tcttctttgcgggagaacata |
| 134 | gagaacatacgatccgtaact |
| 135 | agaacatacgatccgtaacta |
| 136 | ctgcgagaagcgggaagaatt |
| 137 | tgcgagaagcgggaagaattg |
| 138 | cgagaagcgggaagaattgca |

SEQ ID NO: 139-168, 234-235: Oligos cloned to generate precursor amiRs targeting mouse and human Prmt6 and Lsd1 (see Table 5)

SEQ ID NO: 169: control amiR
aaatgtactgcgcgtggagac
SEQ ID NO: 170: EGFP forward primer
gtcctgctggagttcgtg
SEQ ID NO: 171-173: mature amiRs targeting hPRMT6 (5′->3′):

| SEQ ID NO: | Mature amiR |
|---|---|
| 171 | agcacgcuguauccguuguug |
| 172 | auaauaagcacgcuguauccg |
| 173 | uaaggugugaggugucaggga |

SEQ ID NO: 174-176: mature amiRs targeting hLSD1 (5′->3′):

| SEQ ID NO: | Mature amiR |
|---|---|
| 174 | auaacugugaacucgguggac |
| 175 | ugacgcucuaaauaacuguga |
| 176 | uuaggaaccuugacaguguca |

SEQ ID NO: 177-179: precursors of amiRs targeting hPRMT6 (5′->3′);
targeting sequence is underlined.

| SEQ ID NO: | amiR precursor |
|---|---|
| 177 | agcacgcuguauccguuguugguuuuggccacugacugaccaacaacguacagcgugcu |
| 178 | auaauaagcacgcuguauccgguuuuggccacugacugaccggauacagugcuuauuau |
| 179 | uaaggugugaggugucagggaguuuuggccacugacugacucccugaccucacaccuua |

SEQ ID NO: 180-182: precursors of amiRs targeting hLSD1(5′->3′); targeting
sequence is underlined.

| SEQ ID NO: | amiR precursor |
|---|---|
| 180 | auaacugugaacucgguggacguuuuggccacugacugacguccaccguucacaguuau |
| 181 | ugacgcucuaaauaacugugaguuuuggccacugacugacucacaguuuuagagcguca |
| 182 | uuaggaaccuugacagugucaguuuuggccacugacugacugacacugaagguuccuaa |

SEQ ID NO: 183-185 source of amiRs targeting hPRMT6 (5′->3′); targeting
sequence is underlined.

| SEQ ID NO: | amiR source |
|---|---|
| 183 | agcacgctgtatccgttgttggttttggccactgactgaccaacaacgtacagcgtgct |
| 184 | ataataagcacgctgtatccggttttggccactgactgaccggatacagtgcttattat |
| 185 | taaggtgtgaggtgtcagggagttttggccactgactgactccctgacctcacacctta |

SEQ ID NO: 186-188 source of amiRs targeting hLSD1 (5′->3′); targeting
sequence is underlined.

| | |
|---|---|
| SEQ ID NO: | amiR source |
| 186 | ataactgtgaactcggtggacgttttggccactgactgacgtccaccgttcacagttat |
| 187 | tgacgctctaaataactgtgagttttggccactgactgactcacagttttagagcgtca |
| 188 | ttaggaaccttgacagtgtcagttttggccactgactgactgacactgaaggttcctaa |

SEQ ID NO: 189-224 RT-PCR primers

| Primers | | |
|---|---|---|
| mouse genes | SEQ ID NO: | 5′-3′ |
| Prmt6 for | 189 | agtccatgctgagctccgt |
| Prmt6 rev | 190 | tccatgcagctcatatcca |
| Lsd1 for | 191 | tggaactggccaagatcaag |
| Lsd1 rev | 192 | gcttctagcaaccggttaaattc |
| Actin for | 193 | gacaggatgcagaaggagattactg |
| Actin rev | 194 | ctcaggaggagcaatgatcttgat |
| AR for | 195 | gcccgaatgcaaaggtctt |
| AR rev | 196 | tggcgtaacctcccttgaaa |
| Musk for | 197 | atcaccacgcctcttgaaac |
| Musk rev | 198 | tgtcttccacgctcagaatc |
| Myog for | 199 | cttgctcagctccctcaac |
| Myog rev | 200 | tgggagttgcattcactgg |
| Ncam for | 201 | acaatgctgcgaactaagga |
| Ncam rev | 202 | tgccacttgacacagga |
| Cdkn1a for | 203 | aggaccacgtggccttgtc |
| Cdkn1a rev | 204 | ttttcggccctgagatgttc |
| Fkbp4 for | 205 | gaccgagtctttgtccactacac |
| Fkbp4 rev | 206 | atcccaagccttgatgacctcc |
| Fkbp5 for | 207 | aaaggacaatgactactgatgagg |
| Fkbp5 rev | 208 | ctgacaacatccctttgtagtggac |
| Tp53 for | 209 | gggacgggacagctttgag |
| Tp53 rev | 210 | aggacttcctttttgcggaaa |
| Ezh2 for | 211 | catacgctcttctgtcgacgatg |
| Ezh2 rev | 212 | acactgtggtccacaaggcttg |
| Primers | | |
| human genes | SEQ ID NO: | 5′-3′ |
| Prmt6 for | 213 | gagtgctactcggacgtttc |
| Prmt6 rev | 214 | aggatacccaggcggtag |
| Lsd1 for | 215 | agtgagcctgaagaaccatc |
| Lsd1 rev | 216 | tctgttgtggtccactgataat |
| AR for | 217 | tgggagagagacagcttgta |
| AR rev | 218 | agtactgaatgacagccatctg |
| ATPA2 for | 219 | taccgaattgaagggtctttct |
| ATPA2 rev | 220 | cgacattgactttctgtcacg |
| Cdknla for | 221 | aagaccatgtggacctgtca |
| Cdknla rev | 222 | ggcttcctcttggagaagat |
| Actin for | 223 | ggacttcgagcaagagatgg |
| Actin rev | 224 | agcactgtgttggcgtacag |

SEQ ID NO: 225-230 ChIP primers

| Primer | SEQ ID NO: | 5′-3′ |
|---|---|---|
| Neg_For | 225 | tccctgtgcccaaagagtag |
| Neg_Rev | 226 | ccctgaagaatgaggacgag |
| Lsd1_promoter_ARE_For | 227 | cagtctggcctacaaagtgag |
| Lsd1_promoter_ARE_Rev | 228 | gattggtttttccgagacagg |
| Prmt6_promoter_ARE_For | 229 | acttgtcatggcgttagagg |
| Prmt6_promoter_ARE_Rev | 230 | gattcgtagtccgcttcctg |

SEQ ID NO: 231
pcDNA6.2-GW/EmGFP-miR (BLOCK-iT ™ Pol II miR RNAi Expression Vector)
gttgacattgattattgactagttattaatagtaatcaattacggggtcattagttcatagcccatatat
ggagttccgcgttacataacttacggtaaatggcccgcctggctgaccgcccaacgacccccgcccattg
acgtcaataatgacgtatgttcccatagtaacgccaatagggactttccattgacgtcaatgggtggagt
atttacggtaaactgcccacttggcagtacatcaagtgtatcatatgccaagtacgccccctattgacgt
caatgacggtaaatggcccgcctggcattatgcccagtacatgaccttatgggactttcctacttggcag
tacatctacgtattagtcatcgctattaccatggtgatgcggttttggcagtacatcaatgggcgtggat
agcggtttgactcacggggatttccaagtctccaccccattgacgtcaatgggagtttgttttggcacca
aaatcaacgggactttccaaaatgtcgtaacaactccgccccattgacgcaaatgggcggtaggcgtgta
cggtgggaggtctatataagcagagctctctggctaactagagaacccactgcttactggcttatcgaaa
ttaatacgactcactatagggagtcccaagctggctagttaagctatcaacaagtttgtacaaaaaagca
ggctttaaaaccatggtgagcaagggcgaggagctgttcaccggggtggtgcccatcctggtcgagctgg
acggcgacgtaaacggccacaagttcagcgtgtccggcgagggcgagggcgatgccacctacggcaagct
gaccctgaagttcatctgcaccaccggcaagctgcccgtgccctggcccaccctcgtgaccaccttcacc
tacggcgtgcagtgcttcgcccgctaccccgaccacatgaagcagcacgacttcttcaagtccgccatgc
ccgaaggctacgtccaggagcgcaccatcttcttcaaggacgacggcaactacaagacccgcgccgaggt
gaagttcgagggcgacaccctggtgaaccgcatcgagctgaagggcatcgacttcaaggaggacggcaac
atcctggggcacaagctggagtacaactacaacagccacaaggtctatatcaccgccgacaagcagaaga
acggcatcaaggtgaacttcaagacccgccacaacatcgaggacggcagcgtgcagctcgccgaccacta
ccagcagaacacccccatcggcgacggccccgtgctgctgcccgacaaccactacctgagcacccagtcc
gccctgagcaaagaccccaacgagaagcgcgatcacatggtcctgctggagttcgtgaccgccgccggga
tcactctcggcatggacgagctgtacaagtaagctaagcacttcgtggccgtcgatcgtttaaagggagg
tagtgagtcgaccagtggatcctggaggcttgctgaaggctgtatgctcaggacacaaggcctgttacta
gcactcacatggaacaaatggcccagatctggccgcactcgagatatctagacccagctttcttgtacaa
agtggttgatctagagggcccgcggttcgctgatgggggaggctaactgaaacacggaaggagacaatac
cggaaggaacccgcgctatgacggcaataaaaagacagaataaaacgcacgggtgttgggtcgtttgttc
ataaacgcggggttcggtcccagggctggcactctgtcgataccccaccgtgaccccattggggccaata
cgcccgcgtttcttccttttccccaccccaccccccaagttcgggtgaaggcccagggctcgcagccaac
gtcggggcggcaggccctgccatagcatcccctatagtgagtcgtattacatggtcatagctgtttcctg
gcagctctggcccgtgtctcaaaatctctgatggatctgcgcagctggggctctagggggtatccccacg
cgccctgtagcggcgcattaagcgcggcgggtgtggtggttacgcgcagcgtgaccgctacacttgccag
cgccctagcgcccgctcctttcgctttcttcccttcctttctcgccacgttcgccggctttccccgtcaa
gctctaaatcgggggctccctttagggttccgatttagtgctttacggcacctcgaccccaaaaaacttg
attagggtgatggttcacgtagtgggccatcgccctgatagacggtttttcgccctttgacgttggagtc
cacgttctttaatagtggactcttgttccaaactggaacaacactcaaccctatctcggtctattctttt
gatttataagggattttgccgatttcggcctattggttaaaaaatgagctgatttaacaaaaatttaacg
cgaattaattctgtggaatgtgtgtcagttagggtgtggaaagtccccaggctccccagcaggcagaagt
atgcaaagcatgcatctcaattagtcagcaaccaggtgtggaaagtccccaggctccccagcaggcagaa
gtatgcaaagcatgcatctcaattagtcagcaaccatagtcccgcccctaactccgcccatcccgcccct
aactccgcccagttccgcccattctccgccccatggctgactaattttttttatttatgcagaggccgag
gccgcctctgcctctgagctattccagaagtagtgaggaggcttttttggaggcctaggcttttgcaaaa
agctcccgggagcttgtatatccattttcggatctgatcagcacgtgttgacaattaatcatcggcatag
tatatcggcatagtataatacgacaaggtgaggaactaaaccatggccaagcctttgtctcaagaagaat
ccaccctcattgaaagagcaacggctacaatcaacagcatccccatctctgaagactacagcgtcgccag
cgcagctctctctagcgacggccgcatcttcactggtgtcaatgtatatcattttactgggggaccttgt
gcagaactcgtggtgctgggcactgctgctgctgcggcagctggcaacctgacttgtatcgtcgcgatcg
gaaatgagaacaggggcatcttgagcccctgcggacggtgccgacaggtgcttctcgatctgcatcctgg
gatcaaagccatagtgaaggacagtgatggacagccgacggcagttgggattcgtgaattgctgccctct
ggttatgtgtgggagggctaagcacttcgtggccgaggagcaggactgacacgtgctacgagatttcgat
tccaccgccgccttctatgaaaggttgggcttcggaatcgttttccgggacgccggctggatgatcctcc
agcgcggggatctcatgctggagttcttcgcccaccccaacttgtttattgcagcttataatggttacaa
ataaagcaatagcatcacaaatttcacaaataaagcatttttttcactgcattctagttgtggtttgtcc
aaactcatcaatgtatcttatcatgtctgtataccgtcgctcttccgctgcttcctcgctcactgactcg
ctgcgctcggtcgttcggctgcggcgagcggtatcagctcactcaaaggcggtaatacggttatccacag
aatcaggggataacgcaggaaagaacatgtgagcaaaaggccagcaaaaggccaggaaccgtaaaaaggc
cgcgttgctggcgtttttccataggctccgcccccctgacgagcatcacaaaaatcgacgctcaagtcag
aggtggcgaaacccgacaggactataaagataccaggcgtttccccctggaagctccctcgtgcgctctc
ctgttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaaccc
cccgttcagcccgaccgctgcgccttatccggtaactatcgtcttgagtccaacccggtaagacacgact
tatcgccactggcagcagccactggtaacaggattagcagagcgaggtatgtaggcggtgctacagagtt
cttgaagtggtggcctaactacggctacactagaagaacagtatttggtatctgcgctctgctgaagcca
gttaccttcggaaaaagagttggtagctcttgatccggcaaacaaaccaccgctggtagcggtggttttt
ttgtttgcaagcagcagattacgcgcagaaaaaaaggatctcaagaagatcctttgatcttttctacggg
gtctgacgctcagtggaacgacgcgtaactcacgttaagggattttggtcatgggtggctcgacgagggt
tatttgccgactaccttggtgatctcgcctttcacgtagtggacaaattcttccaactgatctgcgcgcg
aggccaagcgatcttcttcttgtccaagataagcctgtctagcttcaagtatgacgggctgatactgggc
cggcaggcgctccattgcccagtcggcagcgacatccttcggcgcgattttgccggttactgcgctgtac
caaatgcgggacaacgtaagcactacatttcgctcatcgccagcccagtcgggcggcgagttccatagcg
ttaaggtttcatttagcgcctcaaatagatcctgttcaggaaccggatcaaagagttcctccgccgctgg
acctaccaaggcaacgctatgttctcttgcttttgtcagcaagatagccagatcaatgtcgatcgtggct
ggctcgaagatacctgcaagaatgtcattgcgctgccattctccaaattgcagttcgcgcttagctggat
aacgccacggaatgatgtcgtcgtgcacaacaatggtgacttctacagcgcggagaatctcgctctctcc
aggggaagccgaagtttccaaaaggtcgttgatcaaagctcgccgcgttgtttcatcaagccttacggtc
accgtaaccagcaaatcaatatcactgtgtggcttcaggccgccatccactgcggagccgtacaaatgta
cggccagcaacgtcggttcgagatggcgctcgatgacgccaactacctctgatagttgagtcgatacttc
ggcgatcaccgcttccctcataatgtttaactttgttttagggcgactgccctgctgcgtaacatcgttg
ctgctccataacatcaaacatcgacccacggcgtaacgcgcttgctgcttggatgcccgaggcatagact
gtaccccaaaaaaacagtcataacaagccatgaaaaccgccactgcgccgttaccaccgctgcgttcggt
caaggttctggaccagttgcgtgagcgcatacgctacttgcattacagcttacgaaccgaacaggcttat
gtccactgggttcgtgccttcatccgtttccacggtgtgcgtcacccggcaaccttgggtagcagcgaag
tcgaggcatttctgtcctggctggtctagaattgcatgaagaatctgcttagggttaggcgttttgcgct
gcttcgcgatgtacgggccagatatacgc
SEQ ID NO: 232 shPRMT6 #1
caccggcauucugagcaucuu.
SEQ ID NO: 233 shPRMU6 #2
cgcauacuucugcgcuacaaa
SEQ ID NO: 236-334 gRNA guide sequences targeting human PRMT6

| SEQ ID NO: | 5′-3′ |
|---|---|
| 236 | gcuguacuacgagugcuacu |
| 237 | ggcaagacgguacuggacgu |
| 238 | acugcgaggcaagacgguac |
| 239 | gauacccaggcgguaggcau |
| 240 | ggugcaaguggagcaagaca |
| 241 | cagauggcaaagccaugcau |
| 242 | uauggcucggcgcccaugca |
| 243 | agccaggugaagcagcacua |
| 244 | gcccauccacucgcucacga |
| 245 | uccuccacgcgcgaaccaag |
| 246 | ggcagcacugcgaggcaaga |
| 247 | gccaucgugagcgaguggau |
| 248 | ccgaaggauacccaggcggu |
| 249 | ggcaacucuacagucuccac |
| 250 | gggggcuaugaagagcucgg |
| 251 | aucguugugcagggauuguc |
| 252 | cacucggagaucguugugca |
| 253 | ugugcagggauuguccggcg |
| 254 | acgauggcauccaccuguuc |
| 255 | guggcccaugagacagcgcg |
| 256 | gcgaccccggaggacuaagc |
| 257 | ccaggcgguaggcaucggug |
| 258 | uggaugccaucgugagcgag |
| 259 | cgguagaggccagcgccauc |
| 260 | gcagugcugcccaguuccga |
| 261 | cccaguuccgaaggauaccc |
| 262 | ccuaccgccuggguauccuu |
| 263 | ccuggguauccuucggaacu |
| 264 | cgcaccgaugccuaccgccu |
| 265 | cguucccgcuuaguccuccg |
| 266 | ucagccacuugguucgcgcg |
| 267 | ggcuaugaagagcucggcgg |
| 268 | acagaagaugcucagaaugc |
| 269 | gugagcgaguggaugggcua |
| 270 | ccugcacaacgaucuccgag |
| 271 | ucagcgaccagaugcuggaa |
| 272 | acgguacuggacgugggcgc |
| 273 | agcgcguggcgaagcccucc |
| 274 | cccggccacucacuggaaac |
| 275 | gggagguggugcgguucaac |
| 276 | ggccgggugaaaaggcgagg |
| 277 | ucuacagucuccacuggucc |
| 278 | cagcuagagcucucccgcgc |
| 279 | guuucuccgacuccccucca |
| 280 | ccggcgcguguacgcgguag |
| 281 | guggaggacggagcucagca |
| 282 | gugucuugcuccacuugcac |
| 283 | aguccuccggggucgcucca |
| 284 | ucuagcugagcaaagcgcug |
| 285 | guggacaugagcugccugga |
| 286 | ccagauggcaaagccaugca |
| 287 | cgaaccaaguggcugaagga |
| 288 | guugaaccgcaccaccuccc |
| 289 | aaggcgagguggacagcacc |
| 290 | gcgcugucucaugggccacu |
| 291 | augaagagcucggcggaggc |
| 292 | ggccagcgccaucuggcaac |
| 293 | caggcgacggggguuguccc |
| 294 | caggagcuggaggccggagu |
| 295 | ucugagcaucuucugugccc |
| 296 | cggaacugggcagcacugcg |
| 297 | aggcgagguggacagcacca |
| 298 | cggggcccggcgcguguacg |
| 299 | gguccacgaggagaugaucg |
| 300 | ccaggcgccauuccagcauc |
| 301 | ccccaucagcgaccagaugc |
| 302 | aguggagacuguagaguugc |
| 303 | gcagcacgcgcaggcgacgg |
| 304 | aggggagucggagaaacccc |
| 305 | ucaccuggcuccagaagccc |
| 306 | gaagaccaaagacuuugcca |
| 307 | ggugaagcagcacuauggug |
| 308 | uguagcgcagcagcacgcgc |
| 309 | cuggacgugggcgcgggcac |
| 310 | gcgcuacaaagugggagacc |
| 311 | gugcugcugcgcuacaaagu |
| 312 | gcgaaccaaguggcugaagg |
| 313 | ggcgagguggacagcaccag |
| 314 | ccaggugaccuucccuggag |
| 315 | gaaggucaccuggaaccaga |
| 316 | aguuccgaaggauacccagg |
| 317 | gcagcagcacgcgcaggcga |
| 318 | ccgagccauagcagcugcag |
| 319 | guucaacgggcuggaggacc |
| 320 | gguccgcgaucaucuccucg |
| 321 | uguggacaugagcugccugg |
| 322 | agcucagcauggacucgugc |
| 323 | accgcccuccuucagccacu |
| 324 | auuguccggcgaggacgugc |
| 325 | ccgggccagcacguccucgc |
| 326 | aggcaacgcucaguccucca |
| 327 | cuccagcuccugcuccaagc |
| 328 | gcaggagcuggaggccggag |
| 329 | cagcaucuggucgcugaugg |
| 330 | gaugggggcuaugaagagcu |
| 331 | accaaguggcugaaggaggg |
| 332 | uuggagcaggagcuggaggc |
| 333 | gagggaacugaagaggaaga |
| 334 | uuccaggugaccuucccugg |

SEQ ID NO: 335-431 gRNA guide sequences targeting human PRMT6

| SEQ ID | |
|---|---|
| NO: | 5′-3′ |
| 335 | agugcgacagguucgcuaca |
| 336 | ugugguccacugauaauauc |
| 337 | ggauacuacaacucacccga |
| 338 | ugguagcagugcaguaccuc |
| 339 | uauaaggugcuucuaauugu |
| 340 | gccacuagugccaacaguau |
| 341 | ggaucugaccgcccuaugca |
| 342 | uguauaccacaccuugcaua |
| 343 | aagauguagcuucuagcaac |
| 344 | uagggcaagcuaccuuguua |
| 345 | ucuuagugaaaagcaaacac |
| 346 | uuucugaaacaggaucgugu |
| 347 | auguauaccacaccuugcau |
| 348 | acaucugcaguccaaaggau |
| 349 | gggacacaggcuuauuauug |
| 350 | auaagugacgaugugauugu |
| 351 | gggauuuggcaaccuuaaca |
| 352 | gcaguccaaaggaugggauu |
| 353 | agugccaacaguauuggagc |
| 354 | ucaacuucggcaucuauaag |
| 355 | agaugauccugcagcaacau |
| 356 | aguagccauuccuuacuguc |
| 357 | acacacuacuuacuguuaua |
| 358 | aagaugagcagauugaacau |
| 359 | gaacucggcaguaauaucuc |
| 360 | aacaucugcaguccaaagga |
| 361 | ugguagagcaagaguuuaac |
| 362 | gaauaagagccccgagccca |
| 363 | auaaauaaagguuugacucg |
| 364 | ugaccgcccuaugcaaggug |
| 365 | ucggcaguaauaucucuggg |
| 366 | ggucagcaaacaaguaaaua |
| 367 | aagguaauuauuauaggcuc |
| 368 | gaaggcauuuaccuuuauag |
| 369 | aagaaacuguggugucucgu |
| 370 | accaagaccuguuaccacca |
| 371 | auacuguuggcacuaguggc |
| 372 | ugcccgaacaaauugacacu |
| 373 | aggagaagcugcugguauca |
| 374 | agguaauuauuauaggcucu |
| 375 | agugagccugaagaaccauc |
| 376 | aggagguccuuacuugguag |
| 377 | aagugagccugaagaaccau |
| 378 | uucaagacgacaguucugga |
| 379 | auagguagaguacagagaga |
| 380 | ugaaauggaaacuuacugug |
| 381 | aaugccuggccaagggacac |
| 382 | ggaaaacaucugcaguccaa |
| 383 | ggcgcaagcgggcgaaggua |
| 384 | ggucaugaggaagucggcuc |
| 385 | ucguuggcgugcugaucccu |
| 386 | gagagguggcacaaacugaa |
| 387 | caaacaaguaaauauggaac |
| 388 | ucgacuuacagcuuguccgu |
| 389 | cuuagugaaaagcaaacaca |
| 390 | aggagggaauccuauggcug |
| 391 | aacucggcaguaauaucucu |
| 392 | aguuggaagcgaauccccca |
| 393 | agagccgacuuccucaugac |
| 394 | gagcaccaugcacuguggcu |
| 395 | uggaccacaacagacccaga |
| 396 | aggccuucugcuaaagccac |
| 397 | cucuucuggaaccucuauaa |
| 398 | uaggagguccuuacuuggua |
| 399 | auaccacaccuugcauaggg |
| 400 | acugauaauaucuggaaaac |
| 401 | agccaccugacaguaaggaa |
| 402 | ucauccggucaugaggaagu |
| 403 | gguggcacaaacugaacggc |
| 404 | gaugccuggccauucucaaa |
| 405 | aauaauacucaucuucugag |
| 406 | aguucucuguacccuucccc |
| 407 | cugcuucuugagaagucauc |
| 408 | gaugaugacuuugaguucac |
| 409 | ugugucccuuggccaggcau |
| 410 | aaugcagcugcaguuguggu |
| 411 | agcugaucuuggagccaugg |
| 412 | gcuucagcacacccagggga |
| 413 | aacuuccaaugccuggccaa |
| 414 | guaggagguccuuacuuggu |
| 415 | ggaauagcagagacuccgga |
| 416 | ccgcaagaaagagccuccgc |
| 417 | gcugcugcuucagcacaccc |
| 418 | ugguuaaaaggacacuguca |
| 419 | gagacagacaaauacuugau |
| 420 | gugccuguggcuuuagcaga |
| 421 | cuucagcacacccaggggaa |
| 422 | guucucuguacccuuccccu |
| 423 | ugcugcuucagcacacccag |
| 424 | gaagaagauagugaaaacuc |
| 425 | aguucuggaggguauggaga |
| 426 | cugcugcuucagcacaccca |
| 427 | uccucagguggggcuugagg |
| 428 | gaauagcagagacuccggag |
| 429 | gaagcgaaucccccaaggua |
| 430 | aggcucuggggucucaggcu |
| 431 | aaguccuccugcuugcccug |

SEQ ID NO: 432 (PRMT6 target of ASO1)
tgtactacgagtgcta
SEQ ID NO: 433 (PRMT6 target of ASO2)
tgcacgagtccatgct
SEQ ID NO: 434 (LSD1 target of ASO3)
tactgtgcttgtccac
SEQ ID NO: 435 (LSD1 target of ASO4)
ctatgtagctgatcttg
SEQ ID NO: 436 (ASO1)
+Ts+As+GsCsAsCsTsCsGsTsAsGsTs+As+Cs+A
SEQ ID NO: 437 (ASO2)
+As+Gs+CsAsTsGsGsAsCsTsCsGsTs+Gs+Cs+A′
SEQ ID NO: 438 (ASO3)
+Gs+Ts+GsGsAsCsAsAsGsCsAsCsAs+Gs+Ts+A
SEQ ID NO: 439 (ASO4)
+Cs+As+AsGsAsTsCsAsGsCsTsAsCsAs+Ts+As+G
SEQ ID NO: 440 (ASO1 unmodified)
tagcactcgtagtaca
SEQ ID NO: 441 (ASO2 unmodified)
agcatggactcgtgca
SEQ ID NO: 442 (ASO3 unmodified)
agcatggactcgtgca
SEQ ID NO: 443 (ASO4 unmodified)
caagatcagctacatag

### REFERENCES

1. Dejager, S., et al. A comprehensive endocrine description of Kennedy's disease revealing androgen insensitivity linked to CAG repeat length. J Clin Endocrinol Metab 87, 3893-3901 (2002).
2. Pandey, U. B., et al. HDAC6 rescues neurodegeneration and provides an essential link between autophagy and the UPS. Nature 447, 859-863 (2007).
3. Yamamoto, T., et al. An open trial of long-term testosterone suppression in spinal and bulbar muscular atrophy. Muscle Nerve 47, 816-822 (2013).
4. Yoshimatsu, M., et al. Dysregulation of PRMT1 and PRMT6, Type I arginine methyltransferases, is involved in various types of human cancers. Int J Cancer 128, 562-573 (2011).
5. Nicoletti L, Paoletti C, Tarricone G, Andreana I, Stella B, Arpicco S, Divieto C, Mattu C, Chiono V. Lipoplexes for effective in vitro delivery of microRNAs to adult human cardiac fibroblasts for perspective direct cardiac cell reprogramming. Nanomedicine 45, 102589 (2022)
6. Metzger, E., et al. LSD1 demethylates repressive histone marks to promote androgen-receptor-dependent transcription. Nature 437, 436-439 (2005).
7. Kahl, P., et al. Androgen receptor coactivators lysine-specific histone demethylase 1 and four and a half LIM domain protein 2 predict risk of prostate cancer recurrence. Cancer Res 66, 11341-11347 (2006).
8. Chivet, M., et al. Polyglutamine-Expanded Androgen Receptor Alteration of Skeletal Muscle Homeostasis and Myonuclear Aggregation Are Affected by Sex, Age and Muscle Metabolism. Cells 9(2020).
9. Pennuto, M. & Basso, M. In Vitro and In Vivo Modeling of Spinal and Bulbar Muscular Atrophy. J Mol Neurosci 58, 365-373 (2016).
10. Zibetti, C., et al. Alternative splicing of the histone demethylase LSD1/KDM1 contributes to the modulation of neurite morphogenesis in the mammalian nervous system. J Neurosci 30, 2521-2532 (2010).
11. Mo, K., et al. Microarray analysis of gene expression by skeletal muscle of three mouse models of Kennedy disease/spinal bulbar muscular atrophy. PLoS One 5, e12922 (2010).
12. MacLean, H. E., et al. Impaired skeletal muscle development and function in male, but not female, genomic androgen receptor knockout mice. FASEB J 22, 2676-2689 (2008).
13. Love, M. I., Huber, W. & Anders, S. Moderated estimation of fold change and dispersion for RNA-seq data with DESeq2. Genome Biol 15, 550 (2014).
14. Zhou, Y., et al. Metascape provides a biologist-oriented resource for the analysis of systems-level datasets. Nat Commun 10, 1523 (2019).
15. Parrini, M., et al. Restoring neuronal chloride homeostasis with anti-NKCC1 gene therapy rescues cognitive deficits in a mouse model of Down syndrome. Mol Ther 29, 3072-3092 (2021).

1. Method of treatment of a disease associated with gain-of-function of AR and/or with overexpression of an AR coactivator, said method comprising
administering to a subject in need thereof a therapeutic agent comprising at least one inhibitor of lysine specific demethylase 1 (LSD1) AR coactivator and at least one inhibitor of protein arginine methyltransferase 6 (PRMT6) AR coactivator.

2. The method of treatment of claim 1, wherein the disease associated with gain-of-function of AR and/or with overexpression of an AR coactivator is Spinal-Bulbar Muscular Atrophy (SBMA).

3. The method of treatment of claim 1, wherein the disease associated with gain-of-function of AR and/or with overexpression of an AR coactivator is cancer, preferably prostate cancer, lung cancer, breast cancer, bladder cancer, cervical cancer, or lymphoma.

4. The method of treatment claim 1, wherein the at least one inhibitor of LSD1 is a genetic silencer targeting LSD1 and/or wherein the at least one inhibitor of PRMT6 is a genetic silencer targeting PRMT6.

5. The method of treatment of claim 4, wherein the at least one genetic silencer targeting LSD1 and/or the at least one genetic silencer targeting PRMT6 is selected from: an RNA interfering (RNAi) molecule, or a precursor or source thereof, or an equivalent thereof; a genome editing agent; an antisense oligonucleotide (ASO); or combinations thereof.

6. The method of treatment therapeutic of claim 4, wherein the at least one genetic silencer targeting LSD1 and/or the at least one genetic silencer targeting PRMT6 is an RNAi molecule, or a precursor or source thereof, or an equivalent thereof.

7. The method of treatment of claim 4, wherein the therapeutic agent comprises:
at least one RNAi molecule, or precursor or source thereof, or an equivalent thereof, targeting any one of sequences SEQ ID NO: 54-138 of LSD1, preferably any one of sequences SEQ ID NO: 68, 72 or 90, or a transcript thereof, and
at least one RNAi molecule, or precursor or source thereof, or an equivalent thereof, targeting any one of sequences SEQ ID NO: 3-53 of PRMT6, preferably any one of sequences SEQ ID NO: 18, 21 or 29, or a transcript thereof.

8. The method of treatment of claim 4, wherein the at least one genetic silencer targeting LSD1 and/or the at least one genetic silencer targeting PRMT6 is an RNAi molecule, or a precursor or source thereof, or an equivalent thereof, and wherein:
the at least one RNAi molecule targeting LSD1 is an artificial miRNA (amiR) comprising, or consisting of, sequence SEQ ID NO: 174-176, or an amiR precursor comprising, or consisting of, sequence SEQ ID NO: 180-182, or an amiR source comprising, or consisting of, sequence SEQ ID NO: 186-188, or an equivalent thereof, and/or wherein:
the at least one RNAi molecule targeting PRMT6 is an artificial miRNA (amiR) comprising, or consisting of, sequence SEQ ID NO: 171-173, or an amiR precursor comprising, or consisting of, sequence SEQ ID NO: 177-178, or an amiR source comprising, or consisting, of sequence SEQ ID NO: 183-185, or an equivalent thereof.

9. The method of treatment therapeutic of claim 4, wherein the at least one genetic silencer targeting LSD1 and/or the at least one genetic silencer targeting PRMT6 is a CRISPR/Cas system.

10. The method of treatment therapeutic of claim 4, wherein the at least one genetic silencer targeting LSD1 and/or the at least one genetic silencer targeting PRMT6 is an ASO.

11. The method of treatment of claim 4, wherein the therapeutic agent comprises:
at least one ASO targeting any one of sequences SEQ ID NO: 434 or 435 of LSD1, or a transcript thereof; and
at least one ASO targeting any one of sequences SEQ ID NO: 432 or 433 of PRMT6, or a transcript thereof.

12. The method of treatment of claim 4, wherein the at least one genetic silencer targeting LSD1 and/or the at least one genetic silencer targeting PRMT6 is an ASO and wherein:
the at least one ASO targeting LSD1 is an ASO comprising or consisting of sequence SEQ ID NO: 438, 439, or an equivalent thereof, and/or wherein:
the at least one ASO targeting PRMT6 is an ASO comprising or consisting of sequence SEQ ID NO: of sequence 436, 437, or an equivalent thereof.

13. The method of treatment of claim 1, wherein the therapeutic agent comprises at least one genetic silencer of LSD1 and/or at least one genetic silencer of PRMT6, and further comprises a delivery vehicle for delivering said genetic silencer in a cell; preferably said delivery vehicle being selected from: a viral vector, microspheres, liposomes, lipoplexes, nanoparticles, microparticles, colloidal gold particles, lipopolysaccharides, polypeptides, polysaccharides, collagen, pegylation of viral vehicles, graphene composites, cholesterol conjugates, cyclodextran complexes, or polyethyleneimine polymers; preferably wherein said delivery vehicle is a viral vector, more preferably selected from: an adeno-associated viral vector, a lentiviral vector, an adenoviral vector, a retroviral vector, an alpha viral vector, a vaccinia virus vector, a herpes simplex virus (HSV) vector, a rabies virus vector, and a Sindbis virus vector.

14. The method of treatment of claim 1, wherein the at least one inhibitor of LSD1 is a small molecule inhibitor of LSD1 and/or wherein the at least one inhibitor of PRMT6 is a small molecule inhibitor of PRMT6.

15. The method of treatment of claim 1, wherein the therapeutic agent comprises at least one genetic silencer of LSD1 and at least one small molecule inhibitor of PRMT6.

16. The method of treatment of claim 1, wherein the therapeutic agent comprises at least one small molecule inhibitor of LSD1 and at least one genetic silencer of PRMT6.

17. The method of treatment of claim 1, wherein the therapeutic agent comprises at least one small molecule inhibitor of LSD1 and at least one genetic silencer of PRMT6 and wherein the at least one small molecule inhibitor of LSD1 is a monoamine oxidase inhibitor, preferably phenelzine, tranylcypromine, or mixtures thereof.

18. An artificial miRNA targeting LSD1 and comprising sequence SEQ ID NO: 174-176, or a source or precursor thereof, or an equivalent thereof or targeting PRMRT6 and comprising SEQ ID NO: 171-173 or a source or precursor thereof, or an equivalent thereof.

19. (canceled)

20. An ASO targeting LSD1 and comprising sequence selected from: SEQ ID NO: 438, 439, or an equivalent thereof or targeting PRMRT6 and comprising SEQ ID NO: 436, 437, or an equivalent thereof.

21. (canceled)


SEQUENCE LISTING (443 sequences)

SEQ ID NO: 1 - DNA, 2621 bp, Homo sapiens
  source 1..2621: mol_type = other DNA
gtgccgcgctacgcccgccgggagccgggcagagcggccaagatgtcgcagcccaagaaaagaaagcttgagtcggggggcggcggcgaaggaggggagggaactgaagaggaagatggcgcggagcgggaggcggccctggagcgaccccggaggactaagcgggaacgggaccagctgtactacgagtgctactcggacgtttcggtccacgaggagatgatcgcggaccgcgtccgcaccgatgcctaccgcctgggtatccttcggaactgggcagcactgcgaggcaagacggtactggacgtgggcgcgggcaccggcattctgagcatcttctgtgcccaggccggggcccggcgcgtgtacgcggtagaggccagcgccatctggcaacaggcccgggaggtggtgcggttcaacgggctggaggaccgggtgcacgtcctgccgggaccagtggagactgtagagttgccggaacaggtggatgccatcgtgagcgagtggatgggctacggactcctgcacgagtccatgctgagctccgtcctccacgcgcgaaccaagtggctgaaggagggcggtcttctcctgccggcctccgccgagctcttcatagcccccatcagcgaccagatgctggaatggcgcctgggcttctggagccaggtgaagcagcactatggtgtggacatgagctgcctggagggcttcgccacgcgctgtctcatgggccactcggagatcgttgtgcagggattgtccggcgaggacgtgctggcccggccgcagcgctttgctcagctagagctctcccgcgccggcttggagcaggagctggaggccggagtgggcgggcgcttccgctgcagctgctatggctcggcgcccatgcatggctttgccatctggttccaggtgaccttccctggaggggagtcggagaaacccctggtgctgtccacctcgccttttcacccggccactcactggaaacaggcgctcctctacctgaacgagccggtgcaagtggagcaagacacggacgtttcaggagagatcacgctgctgccctcccgggacaacccccgtcgcctgcgcgtgctgctgcgctacaaagtgggagaccaggaggagaagaccaaagactttgccatggaggactgagcgttgccttttctcccagctacctcccaaagcagcctgacctgcgtgggagaggcgtagcgaggtcggaggggaaagggagatcccacgtgcaagtagggggaatatctcccccttttccctcatagcctctagggagggagagtgacttcattctccatttgaagagattcttctggtgatgtttacttaaaaagtgatccccctcaacaacggatacagcgtgcttattattgggcatttagcctcaaaagcatgtagtaccaagcacttgtatttccgtatattttgtttcgcgggggagtgagggggaagaacacggatgaaaatgtcagtttttgaagggtccatgcacatccctgacacctcacaccttatctaagtctgaagctggggagaaaggggttcatttagacttcatacatttccagtacgactttagtatctctccagagccatattttctcagtccgaattaattccccctccctaggtgcctgtaggctatggtacttcttcctcattgttttctaggtaaacttcactactggtaattaaggggaaggatatgaggaagcagtttaaatagccctgttctcattactctgaccacatacatcatagggtgctaaagttgatgaacacattaatccgttaagtaaaatggactttgtaattgtacagcatacctaagaaactcagaaggtgcatttaagagagagacctgaaagaaatagtatggatttttaaaaattcttgtctctactattataaccaaaaaatatttcttgtatgtcccataaaaatatttgtgtaattcttatgaaacaggctggtagaggaggtttctgagcctagcccaagggcttattcatcaccatgggtaaattatttaaactcacttaattaaggaaaatattttcccagctagaaaagtatactcattctcatttaaactctctcatttggagggatcatgtgagttggcctacttacaagtagtgaaagttcccttttcagttttgttttgttttgttttgtttttctctttcactcagccaaatgtgaaagttgtgaatttaggaaaatcacttgtaatgaagtgtgaatcttgttatcaaatttatttctctgatgtttccttccttatccttgtagccaataaaacattgacattctcacgttttatagatgaggtaaaaagtcttgtgtgctgtgagttataatgcttttgcctttttaatattattagttcttaagtgttacagccccttcagaatataacttcaggacaattcaaactatgcttaatgtatgattttcgagcttctgtatgctaagaaaataggtgtgaaaaactggtgttctgaaatagcctaacatttattgtaattctgaattttctgcccttttattcattgcatattaaagtattagagtataaaaactaa

SEQ ID NO: 2 - DNA, 3085 bp, Homo sapiens
  source 1..3085: mol_type = other DNA
ggcgcgtgcgtacgcgacggcggttggcggcgcgcgggcagcgtgaagcgaggcgaggcaaggcttttcggacccacggagcgacagagcgagcggcccctacggccgtcggcggcccggcggcccgagatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagacagatgcattctaagggaagaggcccatgtgcctgtttctgccatgtaaggaaggctcttctagcaatactagatcccactgagaaaatccaccctggcatctgggctcctgatcagctgatggagctcctgatttgacaaaggagcttgcctcctttgaatgacctagagcacagggaggaacttgtccattagtttggaattgtgttcttcgtaaagactgaggcaagcaagtgctgtgaaataacatcatcttagtcccttggtgtgtggggtttttgttttttttttatattttgagaataaaacttcatataaaattg

SEQ ID NO: 3 - DNA, 21 bp, Homo sapiens
  source 1..21: mol_type = other DNA
gggaactgaagaggaagatgg

SEQ ID NO: 4 - DNA, 21 bp, Homo sapiens
  source 1..21: mol_type = other DNA
aacaggtggatgccatcgtga

SEQ ID NO: 5 - DNA, 21 bp, Homo sapiens
  source 1..21: mol_type = other DNA
tcagcgaccagatgctggaat

SEQ ID NO: 6 - DNA, 21 bp, Homo sapiens
  source 1..21: mol_type = other DNA
caggtgaagcagcactatggt

SEQ ID NO: 7 - DNA, 21 bp, Homo sapiens
  source 1..21: mol_type = other DNA
gtgaagcagcactatggtgtg

SEQ ID NO: 8 - DNA, 21 bp, Homo sapiens
  source 1..21: mol_type = other DNA
actatggtgtggacatgagct

SEQ ID NO: 9 - DNA, 21 bp, Homo sapiens
  source 1..21: mol_type = other DNA
ggagatcgttgtgcagggatt

SEQ ID NO: 10 - DNA, 21 bp, Homo sapiens
  source 1..21: mol_type = other DNA
agatcgttgtgcagggattgt

SEQ ID NO: 11 - DNA, 21 bp, Homo sapiens
  source 1..21: mol_type = other DNA
ccatgcatggctttgccatct

SEQ ID NO: 12 - DNA, 21 bp, Homo sapiens
  source 1..21: mol_type = other DNA
atgcatggctttgccatctgg

SEQ ID NO: 13 - DNA, 21 bp, Homo sapiens
  source 1..21: mol_type = other DNA
tgcatggctttgccatctggt

SEQ ID NO: 14 - DNA, 21 bp, Homo sapiens
  source 1..21: mol_type = other DNA
gcatggctttgccatctggtt

SEQ ID NO: 15 - DNA, 21 bp, Homo sapiens
  source 1..21: mol_type = other DNA
ttgccatctggttccaggtga

SEQ ID NO: 16 - DNA, 21 bp, Homo sapiens
  source 1..21: mol_type = other DNA
tggagcaagacacggacgttt

SEQ ID NO: 17 - DNA, 21 bp, Homo sapiens
  source 1..21: mol_type = other DNA
cacggacgtttcaggagagat

SEQ ID NO: 18 - DNA, 21 bp, Homo sapiens
  source 1..21: mol_type = other DNA
caacaacggatacagcgtgct

SEQ ID NO: 19 - DNA, 21 bp, Homo sapiens
  source 1..21: mol_type = other DNA
caacggatacagcgtgcttat

SEQ ID NO: 20 - DNA, 21 bp, Homo sapiens
  source 1..21: mol_type = other DNA
aacggatacagcgtgcttatt

SEQ ID NO: 21 - DNA, 21 bp, Homo sapiens
  source 1..21: mol_type = other DNA
cggatacagcgtgcttattat

SEQ ID NO: 22 - DNA, 21 bp, Homo sapiens
  source 1..21: mol_type = other DNA
ggatacagcgtgcttattatt

SEQ ID NO: 23 - DNA, 21 bp, Homo sapiens
  source 1..21: mol_type = other DNA
cagcgtgcttattattgggca

SEQ ID NO: 24 - DNA, 21 bp, Homo sapiens
  source 1..21: mol_type = other DNA
cgtgcttattattgggcattt

SEQ ID NO: 25 - DNA, 21 bp, Homo sapiens
  source 1..21: mol_type = other DNA
gcttattattgggcatttagc

SEQ ID NO: 26 - DNA, 21 bp, Homo sapiens
  source 1..21: mol_type = other DNA
attattgggcatttagcctca

SEQ ID NO: 27 - DNA, 21 bp, Homo sapiens
  source 1..21: mol_type = other DNA
gtagtaccaagcacttgtatt

SEQ ID NO: 28 - DNA, 21 bp, Homo sapiens
  source 1..21: mol_type = other DNA
atccctgacacctcacacctt

SEQ ID NO: 29 - DNA, 21 bp, Homo sapiens
  source 1..21: mol_type = other DNA
tccctgacacctcacacctta

SEQ ID NO: 30 - DNA, 21 bp, Homo sapiens
  source 1..21: mol_type = other DNA
ccctgacacctcacaccttat

SEQ ID NO: 31 - DNA, 21 bp, Homo sapiens
  source 1..21: mol_type = other DNA
tgacacctcacaccttatcta

SEQ ID NO: 32 - DNA, 21 bp, Homo sapiens
  source 1..21: mol_type = other DNA
acacctcacaccttatctaag

SEQ ID NO: 33 - DNA, 21 bp, Homo sapiens
  source 1..21: mol_type = other DNA
cacaccttatctaagtctgaa

SEQ ID NO: 34 - DNA, 21 bp, Homo sapiens
  source 1..21: mol_type = other DNA
acaccttatctaagtctgaag

SEQ ID NO: 35 - DNA, 21 bp, Homo sapiens
  source 1..21: mol_type = other DNA
tcatacatttccagtacgact

SEQ ID NO: 36 - DNA, 21 bp, Homo sapiens
  source 1..21: mol_type = other DNA
catacatttccagtacgactt

SEQ ID NO: 37 - DNA, 21 bp, Homo sapiens
  source 1..21: mol_type = other DNA
tccagtacgactttagtatct

SEQ ID NO: 38 - DNA, 21 bp, Homo sapiens
  source 1..21: mol_type = other DNA
tacgactttagtatctctcca

SEQ ID NO: 39 - DNA, 21 bp, Homo sapiens
  source 1..21: mol_type = other DNA
aggtgcctgtaggctatggta

SEQ ID NO: 40 - DNA, 21 bp, Homo sapiens
  source 1..21: mol_type = genomic DNA
cctgtaggctatggtacttct

SEQ ID NO: 41 - DNA, 21 bp, Homo sapiens
  source 1..21: mol_type = other DNA
ggctatggtacttcttcctca

SEQ ID NO: 42 - DNA, 21 bp, Homo sapiens
  source 1..21: mol_type = other DNA
gttctcattactctgaccaca

SEQ ID NO: 43 - DNA, 21 bp, Homo sapiens
  source 1..21: mol_type = other DNA
ttctcattactctgaccacat

SEQ ID NO: 44 - DNA, 21 bp, Homo sapiens
  source 1..21: mol_type = other DNA
actctgaccacatacatcata

SEQ ID NO: 45 - DNA, 21 bp, Homo sapiens
  source 1..21: mol_type = other DNA
acatacatcatagggtgctaa

SEQ ID NO: 46 - DNA, 21 bp, Homo sapiens
  source 1..21: mol_type = other DNA
catacatcatagggtgctaaa

SEQ ID NO: 47 - DNA, 21 bp, Homo sapiens
  source 1..21: mol_type = other DNA
atacatcatagggtgctaaag

SEQ ID NO: 48 - DNA, 21 bp, Homo sapiens
  source 1..21: mol_type = other DNA
acatcatagggtgctaaagtt

SEQ ID NO: 49 - DNA, 21 bp, Homo sapiens
  source 1..21: mol_type = other DNA
catcatagggtgctaaagttg

SEQ ID NO: 50 - DNA, 21 bp, Homo sapiens
  source 1..21: mol_type = other DNA
tcatagggtgctaaagttgat

SEQ ID NO: 51 - DNA, 21 bp, Homo sapiens
  source 1..21: mol_type = other DNA
gggtgctaaagttgatgaaca

SEQ ID NO: 52 - DNA, 21 bp, Homo sapiens
  source 1..21: mol_type = other DNA
gcatacctaagaaactcagaa

SEQ ID NO: 53 - DNA, 21 bp, Homo sapiens
  source 1..21: mol_type = other DNA
cagccaaatgtgaaagttgtg

SEQ ID NO: 54 - DNA, 21 bp, Homo sapiens
  source 1..21: mol_type = other DNA
ggaaactggaatagcagagac

SEQ ID NO: 55 - DNA, 21 bp, Homo sapiens
  source 1..21: mol_type = other DNA
gaaactggaatagcagagact

SEQ ID NO: 56 - DNA, 21 bp, Homo sapiens
  source 1..21: mol_type = other DNA
gacgacagttctggagggtat

SEQ ID NO: 57 - DNA, 21 bp, Homo sapiens
  source 1..21: mol_type = other DNA
acgacagttctggagggtatg

SEQ ID NO: 58 - DNA, 21 bp, Homo sapiens
  source 1..21: mol_type = other DNA
acagttctggagggtatggag

SEQ ID NO: 59 - DNA, 21 bp, Homo sapiens
  source 1..21: mol_type = other DNA
tttccagagccgacttcctca

SEQ ID NO: 60 - DNA, 21 bp, Homo sapiens
  source 1..21: mol_type = other DNA
ttcctcatgaccggatgactt

SEQ ID NO: 61 - DNA, 21 bp, Homo sapiens
  source 1..21: mol_type = other DNA
catgaccggatgacttctcaa

SEQ ID NO: 62 - DNA, 21 bp, Homo sapiens
  source 1..21: mol_type = other DNA
gaccggatgacttctcaagaa

SEQ ID NO: 63 - DNA, 21 bp, Homo sapiens
  source 1..21: mol_type = other DNA
acactgcagttgtggttggat

SEQ ID NO: 64 - DNA, 21 bp, Homo sapiens
  source 1..21: mol_type = other DNA
cactgcagttgtggttggata

SEQ ID NO: 65 - DNA, 21 bp, Homo sapiens
  source 1..21: mol_type = other DNA
ctgcagttgtggttggataat

SEQ ID NO: 66 - DNA, 21 bp, Homo sapiens
  source 1..21: mol_type = other DNA
cagttgtggttggataatcca

SEQ ID NO: 67 - DNA, 21 bp, Homo sapiens
  source 1..21: mol_type = other DNA
tgtccaccgagttcacagtta

SEQ ID NO: 68 - DNA, 21 bp, Homo sapiens
  source 1..21: mol_type = other DNA
gtccaccgagttcacagttat

SEQ ID NO: 69 - DNA, 21 bp, Homo sapiens
  source 1..21: mol_type = other DNA
caccgagttcacagttattta

SEQ ID NO: 70 - DNA, 21 bp, Homo sapiens
  source 1..21: mol_type = other DNA
ccgagttcacagttatttaga

SEQ ID NO: 71 - DNA, 21 bp, Homo sapiens
  source 1..21: mol_type = other DNA
cgagttcacagttatttagag

SEQ ID NO: 72 - DNA, 21 bp, Homo sapiens
  source 1..21: mol_type = other DNA
tcacagttatttagagcgtca

SEQ ID NO: 73 - DNA, 21 bp, Homo sapiens
  source 1..21: mol_type = other DNA
cacagttatttagagcgtcat

SEQ ID NO: 74 - DNA, 21 bp, Homo sapiens
  source 1..21: mol_type = other DNA
agttatttagagcgtcatggt

SEQ ID NO: 75 - DNA, 21 bp, Homo sapiens
  source 1..21: mol_type = other DNA
ttatttagagcgtcatggtct

SEQ ID NO: 76 - DNA, 21 bp, Homo sapiens
  source 1..21: mol_type = other DNA
tatttagagcgtcatggtctt

SEQ ID NO: 77 - DNA, 21 bp, Homo sapiens
  source 1..21: mol_type = other DNA
ggtggacgagttgccacattt

SEQ ID NO: 78 - DNA, 21 bp, Homo sapiens
  source 1..21: mol_type = other DNA
gacgagttgccacatttcgca

SEQ ID NO: 79 - DNA, 21 bp, Homo sapiens
  source 1..21: mol_type = other DNA
ttgccacatttcgcaaaggaa

SEQ ID NO: 80 - DNA, 21 bp, Homo sapiens
  source 1..21: mol_type = other DNA
tgccacatttcgcaaaggaaa

SEQ ID NO: 81 - DNA, 21 bp, Homo sapiens
  source 1..21: mol_type = other DNA
catttcgcaaaggaaactatg

SEQ ID NO: 82 - DNA, 21 bp, Homo sapiens
  source 1..21: mol_type = other DNA
gccatggtggtaacaggtctt

SEQ ID NO: 83 - DNA, 21 bp, Homo sapiens
  source 1..21: mol_type = other DNA
ggtaacaggtcttggagggaa

SEQ ID NO: 84 - DNA, 21 bp, Homo sapiens
  source 1..21: mol_type = other DNA
aggtcttggagggaatcctat

SEQ ID NO: 85 - DNA, 21 bp, Homo sapiens
  source 1..21: mol_type = other DNA
ggaatcctatggctgtggtca

SEQ ID NO: 86 - DNA, 21 bp, Homo sapiens
  source 1..21: mol_type = other DNA
atcctatggctgtggtcagca

SEQ ID NO: 87 - DNA, 21 bp, Homo sapiens
  source 1..21: mol_type = other DNA
caagctgacactgtcaaggtt

SEQ ID NO: 88 - DNA, 21 bp, Homo sapiens
  source 1..21: mol_type = other DNA
aagctgacactgtcaaggttc

SEQ ID NO: 89 - DNA, 21 bp, Homo sapiens
  source 1..21: mol_type = other DNA
ctgacactgtcaaggttccta

SEQ ID NO: 90 - DNA, 21 bp, Homo sapiens
  source 1..21: mol_type = other DNA
tgacactgtcaaggttcctaa

SEQ ID NO: 91 - DNA, 21 bp, Homo sapiens
  source 1..21: mol_type = other DNA
gacactgtcaaggttcctaaa

SEQ ID NO: 92 - DNA, 21 bp, Homo sapiens
  source 1..21: mol_type = other DNA
actgtcaaggttcctaaagag

SEQ ID NO: 93 - DNA, 21 bp, Homo sapiens
  source 1..21: mol_type = other DNA
ctgtcaaggttcctaaagaga

SEQ ID NO: 94 - DNA, 21 bp, Homo sapiens
  source 1..21: mol_type = other DNA
tgtcaaggttcctaaagagaa

SEQ ID NO: 95 - DNA, 21 bp, Homo sapiens
  source 1..21: mol_type = other DNA
gtcaaggttcctaaagagaaa

SEQ ID NO: 96 - DNA, 21 bp, Homo sapiens
  source 1..21: mol_type = other DNA
agagcaagagtttaaccggtt

SEQ ID NO: 97 - DNA, 21 bp, Homo sapiens
  source 1..21: mol_type = other DNA
gagcaagagtttaaccggttg

SEQ ID NO: 98 - DNA, 21 bp, Homo sapiens
  source 1..21: mol_type = other DNA
aagagtttaaccggttgctag

SEQ ID NO: 99 - DNA, 21 bp, Homo sapiens
  source 1..21: mol_type = other DNA
ctccatcagcaatacaaagaa

SEQ ID NO: 100 - DNA, 21 bp, Homo sapiens
  source 1..21: mol_type = other DNA
cagcaatacaaagaagcatct

SEQ ID NO: 101 - DNA, 21 bp, Homo sapiens
  source 1..21: mol_type = other DNA
ctgaagtaaagccacccagag

SEQ ID NO: 102 - DNA, 21 bp, Homo sapiens
  source 1..21: mol_type = other DNA
taaagccacccagagatatta

SEQ ID NO: 103 - DNA, 21 bp, Homo sapiens
  source 1..21: mol_type = other DNA
ccagagatattactgccgagt

SEQ ID NO: 104 - DNA, 21 bp, Homo sapiens
  source 1..21: mol_type = other DNA
cagagatattactgccgagtt

SEQ ID NO: 105 - DNA, 21 bp, Homo sapiens
  source 1..21: mol_type = other DNA
gagatattactgccgagttct

SEQ ID NO: 106 - DNA, 21 bp, Homo sapiens
  source 1..21: mol_type = other DNA
agatattactgccgagttctt

SEQ ID NO: 107 - DNA, 21 bp, Homo sapiens
  source 1..21: mol_type = other DNA
gatattactgccgagttctta

SEQ ID NO: 108 - DNA, 21 bp, Homo sapiens
  source 1..21: mol_type = other DNA
tgaccgccctatgcaaggaat

SEQ ID NO: 109 - DNA, 21 bp, Homo sapiens
  source 1..21: mol_type = other DNA
gccctatgcaaggaatatgat

SEQ ID NO: 110 - DNA, 21 bp, Homo sapiens
  source 1..21: mol_type = other DNA
cctatgcaaggaatatgatga

SEQ ID NO: 111 - DNA, 21 bp, Homo sapiens
  source 1..21: mol_type = other DNA
cagacaaatacttgattggca

SEQ ID NO: 112 - DNA, 21 bp, Homo sapiens
  source 1..21: mol_type = other DNA
ctctctcaactctctccctta

SEQ ID NO: 113 - DNA, 21 bp, Homo sapiens
  source 1..21: mol_type = other DNA
tctctcaactctctcccttaa

SEQ ID NO: 114 - DNA, 21 bp, Homo sapiens
  source 1..21: mol_type = other DNA
taagcactgggatcaggatga

SEQ ID NO: 115 - DNA, 21 bp, Homo sapiens
  source 1..21: mol_type = other DNA
tgggatcaggatgatgacttt

SEQ ID NO: 116 - DNA, 21 bp, Homo sapiens
  source 1..21: mol_type = other DNA
tcaggatgatgactttgagtt

SEQ ID NO: 117 - DNA, 21 bp, Homo sapiens
  source 1..21: mol_type = other DNA
aggatgatgactttgagttca

SEQ ID NO: 118 - DNA, 21 bp, Homo sapiens
  source 1..21: mol_type = other DNA
atgactttgagttcactggca

SEQ ID NO: 119 - DNA, 21 bp, Homo sapiens
  source 1..21: mol_type = other DNA
gtggctttagcagaaggccta

SEQ ID NO: 120 - DNA, 21 bp, Homo sapiens
  source 1..21: mol_type = other DNA
tttagcagaaggcctagacat

SEQ ID NO: 121 - DNA, 21 bp, Homo sapiens
  source 1..21: mol_type = other DNA
tagcagaaggcctagacatta

SEQ ID NO: 122 - DNA, 21 bp, Homo sapiens
  source 1..21: mol_type = other DNA
aaggcctagacattaaactga

SEQ ID NO: 123 - DNA, 21 bp, Homo sapiens
  source 1..21: mol_type = other DNA
aggcctagacattaaactgaa

SEQ ID NO: 124 - DNA, 21 bp, Homo sapiens
  source 1..21: mol_type = other DNA
agcccaaagaaactgtggtgt

SEQ ID NO: 125 - DNA, 21 bp, Homo sapiens
  source 1..21: mol_type = other DNA
cccaaagaaactgtggtgtct

SEQ ID NO: 126 - DNA, 21 bp, Homo sapiens
  source 1..21: mol_type = other DNA
aagaaactgtggtgtctcgtt

SEQ ID NO: 127 - DNA, 21 bp, Homo sapiens
  source 1..21: mol_type = other DNA
agaaactgtggtgtctcgttg

SEQ ID NO: 128 - DNA, 21 bp, Homo sapiens
  source 1..21: mol_type = other DNA
agccgattccacgactcttct

SEQ ID NO: 129 - DNA, 21 bp, Homo sapiens
  source 1..21: mol_type = other DNA
gccgattccacgactcttctt

SEQ ID NO: 130 - DNA, 21 bp, Homo sapiens
  source 1..21: mol_type = other DNA
cgattccacgactcttctttg

SEQ ID NO: 131 - DNA, 21 bp, Homo sapiens
  source 1..21: mol_type = other DNA
cgactcttctttgcgggagaa

SEQ ID NO: 132 - DNA, 21 bp, Homo sapiens
  source 1..21: mol_type = other DNA
actcttctttgcgggagaaca

SEQ ID NO: 133 - DNA, 21 bp, Homo sapiens
  source 1..21: mol_type = other DNA
tcttctttgcgggagaacata

SEQ ID NO: 134 - DNA, 21 bp, Homo sapiens
  source 1..21: mol_type = other DNA
gagaacatacgatccgtaact

SEQ ID NO: 135 - DNA, 21 bp, Homo sapiens
  source 1..21: mol_type = other DNA
agaacatacgatccgtaacta

SEQ ID NO: 136 - DNA, 21 bp, Homo sapiens
  source 1..21: mol_type = other DNA
ctgcgagaagcgggaagaatt

SEQ ID NO: 137 - DNA, 21 bp, Homo sapiens
  source 1..21: mol_type = other DNA
tgcgagaagcgggaagaattg

SEQ ID NO: 138 - DNA, 21 bp, Homo sapiens
  source 1..21: mol_type = other DNA
cgagaagcgggaagaattgca

SEQ ID NO: 139 - DNA, 64 bp, synthetic construct
  source 1..64: mol_type = other DNA
tgctgagcactcgtagtacagctggtgttttggccactgactgacaccagctgctacgagtgct

SEQ ID NO: 140 - DNA, 64 bp, synthetic construct
  source 1..64: mol_type = other DNA
cctgagcactcgtagcagctggtgtcagtcagtggccaaaacaccagctgtactacgagtgctc

SEQ ID NO: 141 - DNA, 64 bp, synthetic construct
  source 1..64: mol_type = other DNA
tgctgcggagtagcactcgtagtacagttttggccactgactgactgtactacgtgctactccg

SEQ ID NO: 142 - DNA, 64 bp, synthetic construct
  source 1..64: mol_type = other DNA
cctgcggagtagcacgtagtacagtcagtcagtggccaaaactgtactacgagtgctactccgc

SEQ ID NO: 143 - DNA, 64 bp, synthetic construct
  source 1..64: mol_type = other DNA
tgctgttcaggatgcctaagcggtaggttttggccactgactgacctaccgctggcatcctgaa

SEQ ID NO: 144 - DNA, 64 bp, synthetic construct
  source 1..64: mol_type = other DNA
cctgttcaggatgccagcggtaggtcagtcagtggccaaaacctaccgcttaggcatcctgaac

SEQ ID NO: 145 - DNA, 64 bp, synthetic construct
  source 1..64: mol_type = other DNA
tgctgtcttcaggatgcctaagcggtgttttggccactgactgacaccgcttacatcctgaaga

SEQ ID NO: 146 - DNA, 64 bp, synthetic construct
  source 1..64: mol_type = other DNA
cctgtcttcaggatgtaagcggtgtcagtcagtggccaaaacaccgcttaggcatcctgaagac

SEQ ID NO: 147 - DNA, 64 bp, synthetic construct
  source 1..64: mol_type = other DNA
tgctgtagcgcagaagtatgcgcagagttttggccactgactgactctgcgcacttctgcgcta

SEQ ID NO: 148 - DNA, 64 bp, synthetic construct
  source 1..64: mol_type = other DNA
cctgtagcgcagaagtgcgcagagtcagtcagtggccaaaactctgcgcatacttctgcgctac

SEQ ID NO: 149 - DNA, 64 bp, synthetic construct
  source 1..64: mol_type = other DNA
tgctgaatcagaccatgttgcctttcgttttggccactgactgacgaaaggcaatggtctgatt

SEQ ID NO: 150 - DNA, 64 bp, synthetic construct
  source 1..64: mol_type = other DNA
cctgaatcagaccattgcctttcgtcagtcagtggccaaaacgaaaggcaacatggtctgattc

SEQ ID NO: 151 - DNA, 64 bp, synthetic construct
  source 1..64: mol_type = other DNA
tgctgtattagattagagtgctcgcggttttggccactgactgaccgcgagcactaatctaata

SEQ ID NO: 152 - DNA, 64 bp, synthetic construct
  source 1..64: mol_type = other DNA
cctgtattagattagtgctcgcggtcagtcagtggccaaaaccgcgagcactctaatctaatac

SEQ ID NO: 153 - DNA, 64 bp, synthetic construct
  source 1..64: mol_type = other DNA
tgctgtttctctgtattctaccttgggttttggccactgactgacccaaggtaatacagagaaa

SEQ ID NO: 154 - DNA, 64 bp, synthetic construct
  source 1..64: mol_type = other DNA
cctgtttctctgtattaccttgggtcagtcagtggccaaaacccaaggtagaatacagagaaac

SEQ ID NO: 155 - DNA, 64 bp, synthetic construct
  source 1..64: mol_type = other DNA
tgctgtttacttgtttgctgacgacagttttggccactgactgactgtcgtcaaaacaagtaaa

SEQ ID NO: 156 - DNA, 64 bp, synthetic construct
  source 1..64: mol_type = other DNA
cctgtttacttgttttgacgacagtcagtcagtggccaaaactgtcgtcagcaaacaagtaaac

SEQ ID NO: 157 - DNA, 64 bp, synthetic construct
  source 1..64: mol_type = other DNA
tgctgagcacgctgtatccgttgttggttttggccactgactgaccaacaacgtacagcgtgct

SEQ ID NO: 158 - DNA, 64 bp, synthetic construct
  source 1..64: mol_type = other DNA
cctgagcacgctgtacgttgttggtcagtcagtggccaaaaccaacaacggatacagcgtgctc

SEQ ID NO: 159 - DNA, 64 bp, synthetic construct
  source 1..64: mol_type = other DNA
tgctgataataagcacgctgtatccggttttggccactgactgaccggatacagtgcttattat

SEQ ID NO: 160 - DNA, 64 bp, synthetic construct
  source 1..64: mol_type = other DNA
cctgataataagcactgtatccggtcagtcagtggccaaaaccggatacagcgtgcttattatc

SEQ ID NO: 161 - DNA, 64 bp, synthetic construct
  source 1..64: mol_type = other DNA
tgctgtaaggtgtgaggtgtcagggagttttggccactgactgactccctgacctcacacctta

SEQ ID NO: 162 - DNA, 64 bp, synthetic construct
  source 1..64: mol_type = other DNA
cctgtaaggtgtgaggtcagggagtcagtcagtggccaaaactccctgacacctcacaccttac

SEQ ID NO: 163 - DNA, 64 bp, synthetic construct
  source 1..64: mol_type = other DNA
tgctgataactgtgaactcggtggacgttttggccactgactgacgtccaccgttcacagttat

SEQ ID NO: 164 - DNA, 64 bp, synthetic construct
  source 1..64: mol_type = other DNA
cctgataactgtgaacggtggacgtcagtcagtggccaaaacgtccaccgagttcacagttatc

SEQ ID NO: 165 - DNA, 64 bp, synthetic construct
  source 1..64: mol_type = other DNA
tgctgtgacgctctaaataactgtgagttttggccactgactgactcacagttttagagcgtca

SEQ ID NO: 166 - DNA, 64 bp, synthetic construct
  source 1..64: mol_type = other DNA
cctgtgacgctctaaaactgtgagtcagtcagtggccaaaactcacagttatttagagcgtcac

SEQ ID NO: 167 - DNA, 64 bp, synthetic construct
  source 1..64: mol_type = other DNA
tgctgttaggaaccttgacagtgtcagttttggccactgactgactgacactgaaggttcctaa

SEQ ID NO: 168 - DNA, 64 bp, synthetic construct
  source 1..64: mol_type = other DNA
cctgttaggaaccttcagtgtcagtcagtcagtggccaaaactgacactgtcaaggttcctaac

SEQ ID NO: 169 - DNA, 21 bp, synthetic construct
  source 1..21: mol_type = other DNA
aaatgtactgcgcgtggagac

SEQ ID NO: 170 - DNA, 18 bp, synthetic construct
  source 1..18: mol_type = other DNA
gtcctgctggagttcgtg

SEQ ID NO: 171 - RNA, 21 nt, synthetic construct
  source 1..21: mol_type = other RNA
agcacgctgtatccgttgttg

SEQ ID NO: 172 - RNA, 21 nt, synthetic construct
  source 1..21: mol_type = other RNA
ataataagcacgctgtatccg

SEQ ID NO: 173 - RNA, 21 nt, synthetic construct
  source 1..21: mol_type = other RNA
taaggtgtgaggtgtcaggga

SEQ ID NO: 174 - RNA, 21 nt, synthetic construct
  source 1..21: mol_type = other RNA
ataactgtgaactcggtggac

SEQ ID NO: 175 - RNA, 21 nt, synthetic construct
  source 1..21: mol_type = other RNA
tgacgctctaaataactgtga

SEQ ID NO: 176 - RNA, 21 nt, synthetic construct
  source 1..21: mol_type = other RNA
ttaggaaccttgacagtgtca

SEQ ID NO: 177 - RNA, 59 nt, synthetic construct
  source 1..59: mol_type = other RNA
agcacgctgtatccgttgttggttttggccactgactgaccaacaacgtacagcgtgct

SEQ ID NO: 178 - RNA, 59 nt, synthetic construct
  source 1..59: mol_type = other RNA
ataataagcacgctgtatccggttttggccactgactgaccggatacagtgcttattat

SEQ ID NO: 179 - RNA, 59 nt, synthetic construct
  source 1..59: mol_type = other RNA
taaggtgtgaggtgtcagggagttttggccactgactgactccctgacctcacacctta

SEQ ID NO: 180 - RNA, 59 nt, synthetic construct
  source 1..59: mol_type = other RNA
ataactgtgaactcggtggacgttttggccactgactgacgtccaccgttcacagttat

SEQ ID NO: 181 - RNA, 59 nt, synthetic construct
  source 1..59: mol_type = other RNA
tgacgctctaaataactgtgagttttggccactgactgactcacagttttagagcgtca

SEQ ID NO: 182 - RNA, 59 nt, synthetic construct
  source 1..59: mol_type = other RNA
ttaggaaccttgacagtgtcagttttggccactgactgactgacactgaaggttcctaa

SEQ ID NO: 183 - DNA, 59 bp, synthetic construct
  source 1..59: mol_type = other DNA
agcacgctgtatccgttgttggttttggccactgactgaccaacaacgtacagcgtgct

SEQ ID NO: 184 - DNA, 59 bp, synthetic construct
  source 1..59: mol_type = other DNA
ataataagcacgctgtatccggttttggccactgactgaccggatacagtgcttattat

SEQ ID NO: 185 - DNA, 59 bp, synthetic construct
  source 1..59: mol_type = other DNA
taaggtgtgaggtgtcagggagttttggccactgactgactccctgacctcacacctta

SEQ ID NO: 186 - DNA, 59 bp, synthetic construct
  source 1..59: mol_type = other DNA
ataactgtgaactcggtggacgttttggccactgactgacgtccaccgttcacagttat

SEQ ID NO: 187 - DNA, 59 bp, synthetic construct
  source 1..59: mol_type = other DNA
tgacgctctaaataactgtgagttttggccactgactgactcacagttttagagcgtca

SEQ ID NO: 188 - DNA, 59 bp, synthetic construct
  source 1..59: mol_type = other DNA
ttaggaaccttgacagtgtcagttttggccactgactgactgacactgaaggttcctaa

SEQ ID NO: 189 - DNA, 19 bp, synthetic construct
  source 1..19: mol_type = other DNA
agtccatgctgagctccgt

SEQ ID NO: 190 - DNA, 19 bp, synthetic construct
  source 1..19: mol_type = other DNA
tccatgcagctcatatcca

SEQ ID NO: 191 - DNA, 20 bp, synthetic construct
  source 1..20: mol_type = other DNA
tggaactggccaagatcaag

SEQ ID NO: 192 - DNA, 23 bp, synthetic construct
  source 1..23: mol_type = other DNA
gcttctagcaaccggttaaattc

SEQ ID NO: 193 - DNA, 25 bp, synthetic construct
  source 1..25: mol_type = other DNA
gacaggatgcagaaggagattactg

SEQ ID NO: 194 - DNA, 24 bp, synthetic construct
  source 1..24: mol_type = other DNA
ctcaggaggagcaatgatcttgat

SEQ ID NO: 195 - DNA, 19 bp, synthetic construct
  source 1..19: mol_type = other DNA
gcccgaatgcaaaggtctt

SEQ ID NO: 196 - DNA, 20 bp, synthetic construct
  source 1..20: mol_type = other DNA
tggcgtaacctcccttgaaa

SEQ ID NO: 197 - DNA, 20 bp, synthetic construct
  source 1..20: mol_type = other DNA
atcaccacgcctcttgaaac

SEQ ID NO: 198 - DNA, 20 bp, synthetic construct
  source 1..20: mol_type = other DNA
tgtcttccacgctcagaatc

SEQ ID NO: 199 - DNA, 19 bp, synthetic construct
  source 1..19: mol_type = other DNA
cttgctcagctccctcaac

SEQ ID NO: 200 - DNA, 19 bp, synthetic construct
  source 1..19: mol_type = other DNA
tgggagttgcattcactgg

SEQ ID NO: 201 - DNA, 20 bp, synthetic construct
  source 1..20: mol_type = other DNA
acaatgctgcgaactaagga

SEQ ID NO: 202 - DNA, 17 bp, synthetic construct
  source 1..17: mol_type = other DNA
tgccacttgacacagga

SEQ ID NO: 203 - DNA, 19 bp, synthetic construct
  source 1..19: mol_type = other DNA
aggaccacgtggccttgtc

SEQ ID NO: 204 - DNA, 20 bp, synthetic construct
  source 1..20: mol_type = other DNA
ttttcggccctgagatgttc

SEQ ID NO: 205 - DNA, 23 bp, synthetic construct
  source 1..23: mol_type = other DNA
gaccgagtctttgtccactacac

SEQ ID NO: 206 - DNA, 22 bp, synthetic construct
  source 1..22: mol_type = other DNA
atcccaagccttgatgacctcc

SEQ ID NO: 207 - DNA, 24 bp, synthetic construct
  source 1..24: mol_type = other DNA
aaaggacaatgactactgatgagg

SEQ ID NO: 208 - DNA, 25 bp, synthetic construct
  source 1..25: mol_type = other DNA
ctgacaacatccctttgtagtggac

SEQ ID NO: 209 - DNA, 19 bp, synthetic construct
  source 1..19: mol_type = other DNA
gggacgggacagctttgag

SEQ ID NO: 210 - DNA, 21 bp, synthetic construct
  source 1..21: mol_type = other DNA
aggacttcctttttgcggaaa

SEQ ID NO: 211 - DNA, 23 bp, synthetic construct
  source 1..23: mol_type = other DNA
catacgctcttctgtcgacgatg

SEQ ID NO: 212 - DNA, 22 bp, synthetic construct
  source 1..22: mol_type = other DNA
acactgtggtccacaaggcttg

SEQ ID NO: 213 - DNA, 20 bp, synthetic construct
  source 1..20: mol_type = other DNA
gagtgctactcggacgtttc

SEQ ID NO: 214 - DNA, 18 bp, synthetic construct
  source 1..18: mol_type = other DNA
aggatacccaggcggtag

SEQ ID NO: 215 - DNA, 20 bp, synthetic construct
  source 1..20: mol_type = other DNA
agtgagcctgaagaaccatc

SEQ ID NO: 216 - DNA, 22 bp, synthetic construct
  source 1..22: mol_type = other DNA
tctgttgtggtccactgataat

SEQ ID NO: 217 - DNA, 20 bp, synthetic construct
  source 1..20: mol_type = other DNA
tgggagagagacagcttgta

SEQ ID NO: 218 - DNA, 22 bp, synthetic construct
  source 1..22: mol_type = other DNA
agtactgaatgacagccatctg

SEQ ID NO: 219 - DNA, 22 bp, synthetic construct
  source 1..22: mol_type = other DNA
taccgaattgaagggtctttct

SEQ ID NO: 220 - DNA, 21 bp, synthetic construct
  source 1..21: mol_type = other DNA
cgacattgactttctgtcacg

SEQ ID NO: 221 - DNA, 20 bp, synthetic construct
  source 1..20: mol_type = other DNA
aagaccatgtggacctgtca

SEQ ID NO: 222 - DNA, 20 bp, synthetic construct
  source 1..20: mol_type = other DNA
ggcttcctcttggagaagat

SEQ ID NO: 223 - DNA, 20 bp, synthetic construct
  source 1..20: mol_type = other DNA
ggacttcgagcaagagatgg

SEQ ID NO: 224 - DNA, 20 bp, synthetic construct
  source 1..20: mol_type = other DNA
agcactgtgttggcgtacag

SEQ ID NO: 225 - DNA, 20 bp, synthetic construct
  source 1..20: mol_type = other DNA
tccctgtgcccaaagagtag

SEQ ID NO: 226 - DNA, 20 bp, synthetic construct
  source 1..20: mol_type = other DNA
ccctgaagaatgaggacgag

SEQ ID NO: 227 - DNA, 21 bp, synthetic construct
  source 1..21: mol_type = other DNA
cagtctggcctacaaagtgag

SEQ ID NO: 228 - DNA, 21 bp, synthetic construct
  source 1..21: mol_type = other DNA
gattggtttttccgagacagg

SEQ ID NO: 229 - DNA, 20 bp, synthetic construct
  source 1..20: mol_type = other DNA
acttgtcatggcgttagagg

SEQ ID NO: 230 - DNA, 20 bp, synthetic construct
  source 1..20: mol_type = other DNA
gattcgtagtccgcttcctg

SEQ ID NO: 231 - DNA, 5699 bp, synthetic construct
  source 1..5699: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagtcccaagctggctagttaagctatcaacaagtttgtacaaaaaagcaggctttaaaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctaagcacttcgtggccgtcgatcgtttaaagggaggtagtgagtcgaccagtggatcctggaggcttgctgaaggctgtatgctcaggacacaaggcctgttactagcactcacatggaacaaatggcccagatctggccgcactcgagatatctagacccagctttcttgtacaaagtggttgatctagagggcccgcggttcgctgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgtgaccccattggggccaatacgcccgcgtttcttccttttccccaccccaccccccaagttcgggtgaaggcccagggctcgcagccaacgtcggggcggcaggccctgccatagcatcccctatagtgagtcgtattacatggtcatagctgtttcctggcagctctggcccgtgtctcaaaatctctgatggatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgctcttccgctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgtaactcacgttaagggattttggtcatgggtggctcgacgagggttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggtctagaattgcatgaagaatctgcttagggttaggcgttttgcgctgcttcgcgatgtacgggccagatatacgc

SEQ ID NO: 232 - RNA, 21 nt, synthetic construct
  source 1..21: mol_type = other RNA
caccggcattctgagcatctt

SEQ ID NO: 233 - RNA, 21 nt, synthetic construct
  source 1..21: mol_type = other RNA
cgcatacttctgcgctacaaa

SEQ ID NO: 234 - DNA, 64 bp, synthetic construct
  source 1..64: mol_type = other DNA
tgctgttgatgagaggtatacatcacgttttggccactgactgacgtgatgtacctctcatcaa

SEQ ID NO: 235 - DNA, 64 bp, synthetic construct
  source 1..64: mol_type = other DNA
cctgttgatgagaggtacatcacgtcagtcagtggccaaaacgtgatgtatacctctcatcaac

SEQ ID NO: 236 - RNA, 20 nt, synthetic construct
  source 1..20: mol_type = other RNA
gctgtactacgagtgctact

SEQ ID NO: 237 - RNA, 20 nt, synthetic construct
  source 1..20: mol_type = other RNA
ggcaagacggtactggacgt

SEQ ID NO: 238 - RNA, 20 nt, synthetic construct
  source 1..20: mol_type = other RNA
actgcgaggcaagacggtac

SEQ ID NO: 239 - RNA, 20 nt, synthetic construct
  source 1..20: mol_type = other RNA
gatacccaggcggtaggcat

SEQ ID NO: 240 - RNA, 20 nt, synthetic construct
  source 1..20: mol_type = other RNA
ggtgcaagtggagcaagaca

SEQ ID NO: 241 - RNA, 20 nt, synthetic construct
  source 1..20: mol_type = other RNA
ggtgcaagtggagcaagaca

SEQ ID NO: 242 - RNA, 20 nt, synthetic construct
  source 1..20: mol_type = other RNA
tatggctcggcgcccatgca

SEQ ID NO: 243 - RNA, 20 nt, synthetic construct
  source 1..20: mol_type = other RNA
agccaggtgaagcagcacta

SEQ ID NO: 244 - RNA, 20 nt, synthetic construct
  source 1..20: mol_type = other RNA
gcccatccactcgctcacga

SEQ ID NO: 245 - RNA, 20 nt, synthetic construct
  source 1..20: mol_type = other RNA
tcctccacgcgcgaaccaag

SEQ ID NO: 246 - RNA, 20 nt, synthetic construct
  source 1..20: mol_type = other RNA
ggcagcactgcgaggcaaga

SEQ ID NO: 247 - RNA, 20 nt, synthetic construct
  source 1..20: mol_type = other RNA
gccatcgtgagcgagtggat

SEQ ID NO: 248 - RNA, 20 nt, synthetic construct
  source 1..20: mol_type = other RNA
ccgaaggatacccaggcggt

SEQ ID NO: 249 - RNA, 20 nt, synthetic construct
  source 1..20: mol_type = other RNA
ggcaactctacagtctccac

SEQ ID NO: 250 - RNA, 20 nt, synthetic construct
  source 1..20: mol_type = other RNA
gggggctatgaagagctcgg

SEQ ID NO: 251 - RNA, 20 nt, synthetic construct
  source 1..20: mol_type = other RNA
atcgttgtgcagggattgtc

SEQ ID NO: 252 - RNA, 20 nt, synthetic construct
  source 1..20: mol_type = other RNA
cactcggagatcgttgtgca

SEQ ID NO: 253 - RNA, 20 nt, synthetic construct
  source 1..20: mol_type = other RNA
tgtgcagggattgtccggcg

SEQ ID NO: 254 - RNA, 20 nt, synthetic construct
  source 1..20: mol_type = other RNA
acgatggcatccacctgttc

SEQ ID NO: 255 - RNA, 20 nt, synthetic construct
  source 1..20: mol_type = other RNA
gtggcccatgagacagcgcg

SEQ ID NO: 256 - RNA, 20 nt, synthetic construct
  source 1..20: mol_type = other RNA
gcgaccccggaggactaagc

SEQ ID NO: 257 - RNA, 20 nt, synthetic construct
  source 1..20: mol_type = other RNA
ccaggcggtaggcatcggtg

SEQ ID NO: 258 - RNA, 20 nt, synthetic construct
  source 1..20: mol_type = other RNA
tggatgccatcgtgagcgag

SEQ ID NO: 259 - RNA, 20 nt, synthetic construct
  source 1..20: mol_type = other RNA
cggtagaggccagcgccatc

SEQ ID NO: 260 - RNA, 20 nt, synthetic construct
  source 1..20: mol_type = other RNA
gcagtgctgcccagttccga

SEQ ID NO: 261 - RNA, 20 nt, synthetic construct
  source 1..20: mol_type = other RNA
cccagttccgaaggataccc

SEQ ID NO: 262 - RNA, 20 nt, synthetic construct
  source 1..20: mol_type = other RNA
cctaccgcctgggtatcctt

SEQ ID NO: 263 - RNA, 20 nt, synthetic construct
  source 1..20: mol_type = other RNA
cctgggtatccttcggaact

SEQ ID NO: 264 - RNA, 20 nt, synthetic construct
  source 1..20: mol_type = other RNA
cgcaccgatgcctaccgcct

SEQ ID NO: 265 - RNA, 20 nt, synthetic construct
  source 1..20: mol_type = other RNA
cgttcccgcttagtcctccg

SEQ ID NO: 266 - RNA, 20 nt, synthetic construct
  source 1..20: mol_type = other RNA
tcagccacttggttcgcgcg

SEQ ID NO: 267 - RNA, 20 nt, synthetic construct
  source 1..20: mol_type = other RNA
ggctatgaagagctcggcgg

SEQ ID NO: 268 - RNA, 20 nt, synthetic construct
  source 1..20: mol_type = other RNA
acagaagatgctcagaatgc

SEQ ID NO: 269 - RNA, 20 nt, synthetic construct
  source 1..20: mol_type = other RNA
gtgagcgagtggatgggcta

SEQ ID NO: 270 - RNA, 20 nt, synthetic construct
  source 1..20: mol_type = other RNA
cctgcacaacgatctccgag

SEQ ID NO: 271 - RNA, 20 nt, synthetic construct
  source 1..20: mol_type = other RNA
tcagcgaccagatgctggaa

SEQ ID NO: 272 - RNA, 20 nt, synthetic construct
  source 1..20: mol_type = other RNA
acggtactggacgtgggcgc

SEQ ID NO: 273 - RNA, 20 nt, synthetic construct
  source 1..20: mol_type = other RNA
agcgcgtggcgaagccctcc

SEQ ID NO: 274 - RNA, 20 nt, synthetic construct
  source 1..20: mol_type = other RNA
cccggccactcactggaaac

SEQ ID NO: 275 - RNA, 20 nt, synthetic construct
  source 1..20: mol_type = other RNA
gggaggtggtgcggttcaac

SEQ ID NO: 276 - RNA, 20 nt, synthetic construct
  source 1..20: mol_type = other RNA
ggccgggtgaaaaggcgagg

SEQ ID NO: 277 - RNA, 20 nt, synthetic construct
  source 1..20: mol_type = other RNA
tctacagtctccactggtcc

SEQ ID NO: 278 - RNA, 20 nt, synthetic construct
  source 1..20: mol_type = other RNA
cagctagagctctcccgcgc

SEQ ID NO: 279 - RNA, 20 nt, synthetic construct
  source 1..20: mol_type = other RNA
gtttctccgactcccctcca

SEQ ID NO: 280 - RNA, 20 nt, synthetic construct
  source 1..20: mol_type = other RNA
ccggcgcgtgtacgcggtag

SEQ ID NO: 281 - RNA, 20 nt, synthetic construct
  source 1..20: mol_type = other RNA
gtggaggacggagctcagca

SEQ ID NO: 282 - RNA, 20 nt, synthetic construct
  source 1..20: mol_type = other RNA
gtgtcttgctccacttgcac

SEQ ID NO: 283 - RNA, 20 nt, synthetic construct
  source 1..20: mol_type = other RNA
agtcctccggggtcgctcca

SEQ ID NO: 284 - RNA, 20 nt, synthetic construct
  source 1..20: mol_type = other RNA
tctagctgagcaaagcgctg

SEQ ID NO: 285 - RNA, 20 nt, synthetic construct
  source 1..20: mol_type = other RNA
gtggacatgagctgcctgga

SEQ ID NO: 286 - RNA, 20 nt, synthetic construct
  source 1..20: mol_type = other RNA
ccagatggcaaagccatgca

SEQ ID NO: 287 - RNA, 20 nt, synthetic construct
  source 1..20: mol_type = other RNA
ccagatggcaaagccatgca

SEQ ID NO: 288 - RNA, 20 nt, synthetic construct
  source 1..20: mol_type = other RNA
cgaaccaagtggctgaagga

SEQ ID NO: 289 - RNA, 20 nt, synthetic construct
  source 1..20: mol_type = other RNA
aaggcgaggtggacagcacc

SEQ ID NO: 290 - RNA, 20 nt, synthetic construct
  source 1..20: mol_type = other RNA
gcgctgtctcatgggccact

SEQ ID NO: 291 - RNA, 20 nt, synthetic construct
  source 1..20: mol_type = other RNA
atgaagagctcggcggaggc

SEQ ID NO: 292 - RNA, 20 nt, synthetic construct
  source 1..20: mol_type = other RNA
ggccagcgccatctggcaac

SEQ ID NO: 293 - RNA, 20 nt, synthetic construct
  source 1..20: mol_type = other RNA
caggcgacgggggttgtccc

SEQ ID NO: 294 - RNA, 20 nt, synthetic construct
  source 1..20: mol_type = other RNA
caggagctggaggccggagt

SEQ ID NO: 295 - RNA, 20 nt, synthetic construct
  source 1..20: mol_type = other RNA
tctgagcatcttctgtgccc

SEQ ID NO: 296 - RNA, 20 nt, synthetic construct
  source 1..20: mol_type = other RNA
cggaactgggcagcactgcg

SEQ ID NO: 297 - RNA, 20 nt, synthetic construct
  source 1..20: mol_type = other RNA
aggcgaggtggacagcacca

SEQ ID NO: 298 - RNA, 20 nt, synthetic construct
  source 1..20: mol_type = other RNA
cggggcccggcgcgtgtacg

SEQ ID NO: 299 - RNA, 20 nt, synthetic construct
  source 1..20: mol_type = other RNA
ggtccacgaggagatgatcg

SEQ ID NO: 300 - RNA, 20 nt, synthetic construct
  source 1..20: mol_type = other RNA
ccaggcgccattccagcatc

SEQ ID NO: 301 - RNA, 20 nt, synthetic construct
  source 1..20: mol_type = other RNA
ccccatcagcgaccagatgc

SEQ ID NO: 302 - RNA, 20 nt, synthetic construct
  source 1..20: mol_type = other RNA
agtggagactgtagagttgc

SEQ ID NO: 303 - RNA, 20 nt, synthetic construct
  source 1..20: mol_type = other RNA
gcagcacgcgcaggcgacgg

SEQ ID NO: 304 - RNA, 20 nt, synthetic construct
  source 1..20: mol_type = other RNA
aggggagtcggagaaacccc

SEQ ID NO: 305 - RNA, 20 nt, synthetic construct
  source 1..20: mol_type = other RNA
tcacctggctccagaagccc

SEQ ID NO: 306 - RNA, 20 nt, synthetic construct
  source 1..20: mol_type = other RNA
gaagaccaaagactttgcca

SEQ ID NO: 307 - RNA, 20 nt, synthetic construct
  source 1..20: mol_type = other RNA
ggtgaagcagcactatggtg

SEQ ID NO: 308 - RNA, 20 nt, synthetic construct
  source 1..20: mol_type = other RNA
tgtagcgcagcagcacgcgc

SEQ ID NO: 309 - RNA, 20 nt, synthetic construct
  source 1..20: mol_type = other RNA
ctggacgtgggcgcgggcac

SEQ ID NO: 310 - RNA, 20 nt, synthetic construct
  source 1..20: mol_type = other RNA
gcgctacaaagtgggagacc

SEQ ID NO: 311 - RNA, 20 nt, synthetic construct
  source 1..20: mol_type = other RNA
gtgctgctgcgctacaaagt

SEQ ID NO: 312 - RNA, 20 nt, synthetic construct
  source 1..20: mol_type = other RNA
gcgaaccaagtggctgaagg

SEQ ID NO: 313 - RNA, 20 nt, synthetic construct
  source 1..20: mol_type = other RNA
ggcgaggtggacagcaccag

SEQ ID NO: 314 - RNA, 20 nt, synthetic construct
  source 1..20: mol_type = other RNA
ccaggtgaccttccctggag

SEQ ID NO: 315 - RNA, 20 nt, synthetic construct
  source 1..20: mol_type = other RNA
gaaggtcacctggaaccaga

SEQ ID NO: 316 - RNA, 20 nt, synthetic construct
  source 1..20: mol_type = other RNA
agttccgaaggatacccagg

SEQ ID NO: 317 - RNA, 20 nt, synthetic construct
  source 1..20: mol_type = other RNA
gcagcagcacgcgcaggcga

SEQ ID NO: 318 - RNA, 20 nt, synthetic construct
  source 1..20: mol_type = other RNA
ccgagccatagcagctgcag

SEQ ID NO: 319 - RNA, 20 nt, synthetic construct
  source 1..20: mol_type = other RNA
gttcaacgggctggaggacc

SEQ ID NO: 320 - RNA, 20 nt, synthetic construct
  source 1..20: mol_type = other RNA
ggtccgcgatcatctcctcg

SEQ ID NO: 321 - RNA, 20 nt, synthetic construct
  source 1..20: mol_type = other RNA
tgtggacatgagctgcctgg

SEQ ID NO: 322 - RNA, 20 nt, synthetic construct
  source 1..20: mol_type = other RNA
agctcagcatggactcgtgc

SEQ ID NO: 323 - RNA, 20 nt, synthetic construct
  source 1..20: mol_type = other RNA
accgccctccttcagccact

SEQ ID NO: 324 - RNA, 20 nt, synthetic construct
  source 1..20: mol_type = other RNA
attgtccggcgaggacgtgc

SEQ ID NO: 325 - RNA, 20 nt, synthetic construct
  source 1..20: mol_type = other RNA
ccgggccagcacgtcctcgc

SEQ ID NO: 326 - RNA, 20 nt, synthetic construct
  source 1..20: mol_type = other RNA
aggcaacgctcagtcctcca

SEQ ID NO: 327 - RNA, 20 nt, synthetic construct
  source 1..20: mol_type = other RNA
ctccagctcctgctccaagc

SEQ ID NO: 328 - RNA, 20 nt, synthetic construct
  source 1..20: mol_type = other RNA
gcaggagctggaggccggag

SEQ ID NO: 329 - RNA, 20 nt, synthetic construct
  source 1..20: mol_type = other RNA
cagcatctggtcgctgatgg

SEQ ID NO: 330 - RNA, 20 nt, synthetic construct
  source 1..20: mol_type = other RNA
gatgggggctatgaagagct

SEQ ID NO: 331 - RNA, 20 nt, synthetic construct
  source 1..20: mol_type = other RNA
accaagtggctgaaggaggg

SEQ ID NO: 332 - RNA, 20 nt, synthetic construct
  source 1..20: mol_type = other RNA
ttggagcaggagctggaggc

SEQ ID NO: 333 - RNA, 20 nt, synthetic construct
  source 1..20: mol_type = other RNA
gagggaactgaagaggaaga

SEQ ID NO: 334 - RNA, 20 nt, synthetic construct
  source 1..20: mol_type = other RNA
ttccaggtgaccttccctgg

SEQ ID NO: 335 - RNA, 20 nt, synthetic construct
  source 1..20: mol_type = other RNA
agtgcgacaggttcgctaca

SEQ ID NO: 336 - RNA, 20 nt, synthetic construct
  source 1..20: mol_type = other RNA
tgtggtccactgataatatc

SEQ ID NO: 337 - RNA, 20 nt, synthetic construct
  source 1..20: mol_type = other RNA
ggatactacaactcacccga

SEQ ID NO: 338 - RNA, 20 nt, synthetic construct
  source 1..20: mol_type = other RNA
tggtagcagtgcagtacctc

SEQ ID NO: 339 - RNA, 20 nt, synthetic construct
  source 1..20: mol_type = other RNA
tataaggtgcttctaattgt

SEQ ID NO: 340 - RNA, 20 nt, synthetic construct
  source 1..20: mol_type = other RNA
gccactagtgccaacagtat

SEQ ID NO: 341 - RNA, 20 nt, synthetic construct
  source 1..20: mol_type = other RNA
ggatctgaccgccctatgca

SEQ ID NO: 342 - RNA, 20 nt, synthetic construct
  source 1..20: mol_type = other RNA
tgtataccacaccttgcata

SEQ ID NO: 343 - RNA, 20 nt, synthetic construct
  source 1..20: mol_type = other RNA
aagatgtagcttctagcaac

SEQ ID NO: 344 - RNA, 20 nt, synthetic construct
  source 1..20: mol_type = other RNA
tagggcaagctaccttgtta

SEQ ID NO: 345 - RNA, 20 nt, synthetic construct
  source 1..20: mol_type = other RNA
tcttagtgaaaagcaaacac

SEQ ID NO: 346 - RNA, 20 nt, synthetic construct
  source 1..20: mol_type = other RNA
tttctgaaacaggatcgtgt

SEQ ID NO: 347 - RNA, 20 nt, synthetic construct
  source 1..20: mol_type = other RNA
atgtataccacaccttgcat

SEQ ID NO: 348 - RNA, 20 nt, synthetic construct
  source 1..20: mol_type = other RNA
acatctgcagtccaaaggat

SEQ ID NO: 349 - RNA, 20 nt, synthetic construct
  source 1..20: mol_type = other RNA
gggacacaggcttattattg

SEQ ID NO: 350 - RNA, 20 nt, synthetic construct
  source 1..20: mol_type = other RNA
ataagtgacgatgtgattgt

SEQ ID NO: 351 - RNA, 20 nt, synthetic construct
  source 1..20: mol_type = other RNA
gggatttggcaaccttaaca

SEQ ID NO: 352 - RNA, 20 nt, synthetic construct
  source 1..20: mol_type = other RNA
gcagtccaaaggatgggatt

SEQ ID NO: 353 - RNA, 20 nt, synthetic construct
  source 1..20: mol_type = other RNA
agtgccaacagtattggagc

SEQ ID NO: 354 - RNA, 20 nt, synthetic construct
  source 1..20: mol_type = other RNA
tcaacttcggcatctataag

SEQ ID NO: 355 - RNA, 20 nt, synthetic construct
  source 1..20: mol_type = other RNA
agatgatcctgcagcaacat

SEQ ID NO: 356 - RNA, 20 nt, synthetic construct
  source 1..20: mol_type = other RNA
agtagccattccttactgtc

SEQ ID NO: 357 - RNA, 20 nt, synthetic construct
  source 1..20: mol_type = other RNA
acacactacttactgttata

SEQ ID NO: 358 - RNA, 20 nt, synthetic construct
  source 1..20: mol_type = other RNA
aagatgagcagattgaacat

SEQ ID NO: 359 - RNA, 20 nt, synthetic construct
  source 1..20: mol_type = other RNA
gaactcggcagtaatatctc

SEQ ID NO: 360 - RNA, 20 nt, synthetic construct
  source 1..20: mol_type = other RNA
aacatctgcagtccaaagga

SEQ ID NO: 361 - RNA, 20 nt, synthetic construct
  source 1..20: mol_type = other RNA
tggtagagcaagagtttaac

SEQ ID NO: 362 - RNA, 20 nt, synthetic construct
  source 1..20: mol_type = other RNA
gaataagagccccgagccca

SEQ ID NO: 363 - RNA, 20 nt, synthetic construct
  source 1..20: mol_type = other RNA
ataaataaaggtttgactcg

SEQ ID NO: 364 - RNA, 20 nt, synthetic construct
  source 1..20: mol_type = other RNA
tgaccgccctatgcaaggtg

SEQ ID NO: 365 - RNA, 20 nt, synthetic construct
  source 1..20: mol_type = other RNA
tcggcagtaatatctctggg

SEQ ID NO: 366 - RNA, 20 nt, synthetic construct
  source 1..20: mol_type = other RNA
ggtcagcaaacaagtaaata

SEQ ID NO: 367 - RNA, 20 nt, synthetic construct
  source 1..20: mol_type = other RNA
aaggtaattattataggctc

SEQ ID NO: 368 - RNA, 20 nt, synthetic construct
  source 1..20: mol_type = other RNA
gaaggcatttacctttatag

SEQ ID NO: 369 - RNA, 20 nt, synthetic construct
  source 1..20: mol_type = other RNA
aagaaactgtggtgtctcgt

SEQ ID NO: 370 - RNA, 20 nt, synthetic construct
  source 1..20: mol_type = other RNA
accaagacctgttaccacca

SEQ ID NO: 371 - RNA, 20 nt, synthetic construct
  source 1..20: mol_type = other RNA
atactgttggcactagtggc

SEQ ID NO: 372 - RNA, 20 nt, synthetic construct
  source 1..20: mol_type = other RNA
tgcccgaacaaattgacact

SEQ ID NO: 373 - RNA, 20 nt, synthetic construct
  source 1..20: mol_type = other RNA
aggagaagctgctggtatca

SEQ ID NO: 374 - RNA, 20 nt, synthetic construct
  source 1..20: mol_type = other RNA
aggtaattattataggctct

SEQ ID NO: 375 - RNA, 20 nt, synthetic construct
  source 1..20: mol_type = other RNA
agtgagcctgaagaaccatc

SEQ ID NO: 376 - RNA, 20 nt, synthetic construct
  source 1..20: mol_type = other RNA
aggaggtccttacttggtag

SEQ ID NO: 377 - RNA, 20 nt, synthetic construct
  source 1..20: mol_type = other RNA
aagtgagcctgaagaaccat

SEQ ID NO: 378 - RNA, 20 nt, synthetic construct
  source 1..20: mol_type = other RNA
ttcaagacgacagttctgga

SEQ ID NO: 379 - RNA, 20 nt, synthetic construct
  source 1..20: mol_type = other RNA
ataggtagagtacagagaga

SEQ ID NO: 380 - RNA, 20 nt, synthetic construct
  source 1..20: mol_type = other RNA
tgaaatggaaacttactgtg

SEQ ID NO: 381 - RNA, 20 nt, synthetic construct
  source 1..20: mol_type = other RNA
aatgcctggccaagggacac

SEQ ID NO: 382 - RNA, 20 nt, synthetic construct
  source 1..20: mol_type = other RNA
ggaaaacatctgcagtccaa

SEQ ID NO: 383 - RNA, 20 nt, synthetic construct
  source 1..20: mol_type = other RNA
ggcgcaagcgggcgaaggta

SEQ ID NO: 384 - RNA, 20 nt, synthetic construct
  source 1..20: mol_type = other RNA
ggtcatgaggaagtcggctc

SEQ ID NO: 385 - RNA, 20 nt, synthetic construct
  source 1..20: mol_type = other RNA
tcgttggcgtgctgatccct

SEQ ID NO: 386 - RNA, 20 nt, synthetic construct
  source 1..20: mol_type = other RNA
gagaggtggcacaaactgaa

SEQ ID NO: 387 - RNA, 20 nt, synthetic construct
  source 1..20: mol_type = other RNA
caaacaagtaaatatggaac

SEQ ID NO: 388 - RNA, 20 nt, synthetic construct
  source 1..20: mol_type = other RNA
tcgacttacagcttgtccgt

SEQ ID NO: 389 - RNA, 20 nt, synthetic construct
  source 1..20: mol_type = other RNA
cttagtgaaaagcaaacaca

SEQ ID NO: 390 - RNA, 20 nt, synthetic construct
  source 1..20: mol_type = other RNA
aggagggaatcctatggctg

SEQ ID NO: 391 - RNA, 20 nt, synthetic construct
  source 1..20: mol_type = other RNA
aactcggcagtaatatctct

SEQ ID NO: 392 - RNA, 20 nt, synthetic construct
  source 1..20: mol_type = other RNA
agttggaagcgaatccccca

SEQ ID NO: 393 - RNA, 20 nt, synthetic construct
  source 1..20: mol_type = other RNA
agagccgacttcctcatgac

SEQ ID NO: 394 - RNA, 20 nt, synthetic construct
  source 1..20: mol_type = other RNA
gagcaccatgcactgtggct

SEQ ID NO: 395 - RNA, 20 nt, synthetic construct
  source 1..20: mol_type = other RNA
tggaccacaacagacccaga

SEQ ID NO: 396 - RNA, 20 nt, synthetic construct
  source 1..20: mol_type = other RNA
aggccttctgctaaagccac

SEQ ID NO: 397 - RNA, 20 nt, synthetic construct
  source 1..20: mol_type = other RNA
ctcttctggaacctctataa

SEQ ID NO: 398 - RNA, 20 nt, synthetic construct
  source 1..20: mol_type = other RNA
taggaggtccttacttggta

SEQ ID NO: 399 - RNA, 20 nt, synthetic construct
  source 1..20: mol_type = other RNA
ataccacaccttgcataggg

SEQ ID NO: 400 - RNA, 20 nt, synthetic construct
  source 1..20: mol_type = other RNA
actgataatatctggaaaac

SEQ ID NO: 401 - RNA, 20 nt, synthetic construct
  source 1..20: mol_type = other RNA
agccacctgacagtaaggaa

SEQ ID NO: 402 - RNA, 20 nt, synthetic construct
  source 1..20: mol_type = other RNA
tcatccggtcatgaggaagt

SEQ ID NO: 403 - RNA, 20 nt, synthetic construct
  source 1..20: mol_type = other RNA
ggtggcacaaactgaacggc

SEQ ID NO: 404 - RNA, 20 nt, synthetic construct
  source 1..20: mol_type = other RNA
gatgcctggccattctcaaa

SEQ ID NO: 405 - RNA, 20 nt, synthetic construct
  source 1..20: mol_type = other RNA
aataatactcatcttctgag

SEQ ID NO: 406 - RNA, 20 nt, synthetic construct
  source 1..20: mol_type = other RNA
agttctctgtacccttcccc

SEQ ID NO: 407 - RNA, 20 nt, synthetic construct
  source 1..20: mol_type = other RNA
ctgcttcttgagaagtcatc

SEQ ID NO: 408 - RNA, 20 nt, synthetic construct
  source 1..20: mol_type = other RNA
gatgatgactttgagttcac

SEQ ID NO: 409 - RNA, 20 nt, synthetic construct
  source 1..20: mol_type = other RNA
tgtgtcccttggccaggcat

SEQ ID NO: 410 - RNA, 20 nt, synthetic construct
  source 1..20: mol_type = other RNA
aatgcagctgcagttgtggt

SEQ ID NO: 411 - RNA, 20 nt, synthetic construct
  source 1..20: mol_type = other RNA
agctgatcttggagccatgg

SEQ ID NO: 412 - RNA, 20 nt, synthetic construct
  source 1..20: mol_type = other RNA
gcttcagcacacccagggga

SEQ ID NO: 413 - RNA, 20 nt, synthetic construct
  source 1..20: mol_type = other RNA
aacttccaatgcctggccaa

SEQ ID NO: 414 - RNA, 20 nt, synthetic construct
  source 1..20: mol_type = other RNA
gtaggaggtccttacttggt

SEQ ID NO: 415 - RNA, 20 nt, synthetic construct
  source 1..20: mol_type = other RNA
ggaatagcagagactccgga

SEQ ID NO: 416 - RNA, 20 nt, synthetic construct
  source 1..20: mol_type = other RNA
ccgcaagaaagagcctccgc

SEQ ID NO: 417 - RNA, 20 nt, synthetic construct
  source 1..20: mol_type = other RNA
gctgctgcttcagcacaccc

SEQ ID NO: 418 - RNA, 20 nt, synthetic construct
  source 1..20: mol_type = other RNA
tggttaaaaggacactgtca

SEQ ID NO: 419 - RNA, 20 nt, synthetic construct
  source 1..20: mol_type = other RNA
gagacagacaaatacttgat

SEQ ID NO: 420 - RNA, 20 nt, synthetic construct
  source 1..20: mol_type = other RNA
gtgcctgtggctttagcaga

SEQ ID NO: 421 - RNA, 20 nt, synthetic construct
  source 1..20: mol_type = other RNA
cttcagcacacccaggggaa

SEQ ID NO: 422 - RNA, 20 nt, synthetic construct
  source 1..20: mol_type = other RNA
gttctctgtacccttcccct

SEQ ID NO: 423 - RNA, 20 nt, synthetic construct
  source 1..20: mol_type = other RNA
tgctgcttcagcacacccag

SEQ ID NO: 424 - RNA, 20 nt, synthetic construct
  source 1..20: mol_type = other RNA
gaagaagatagtgaaaactc

SEQ ID NO: 425 - RNA, 20 nt, synthetic construct
  source 1..20: mol_type = other RNA
agttctggagggtatggaga

SEQ ID NO: 426 - RNA, 20 nt, synthetic construct
  source 1..20: mol_type = other RNA
ctgctgcttcagcacaccca

SEQ ID NO: 427 - RNA, 20 nt, synthetic construct
  source 1..20: mol_type = other RNA
tcctcaggtggggcttgagg

SEQ ID NO: 428 - RNA, 20 nt, synthetic construct
  source 1..20: mol_type = other RNA
gaatagcagagactccggag

SEQ ID NO: 429 - RNA, 20 nt, synthetic construct
  source 1..20: mol_type = other RNA
gaagcgaatcccccaaggta

SEQ ID NO: 430 - RNA, 20 nt, synthetic construct
  source 1..20: mol_type = other RNA
aggctctggggtctcaggct

SEQ ID NO: 431 - RNA, 20 nt, synthetic construct
  source 1..20: mol_type = other RNA
aagtcctcctgcttgccctg

SEQ ID NO: 432 - DNA, 16 bp, Homo sapiens
  source 1..16: mol_type = other DNA
tgtactacgagtgcta

SEQ ID NO: 433 - DNA, 16 bp, Homo sapiens
  source 1..16: mol_type = other DNA
tgcacgagtccatgct

SEQ ID NO: 434 - DNA, 16 bp, Homo sapiens
  source 1..16: mol_type = other DNA
tactgtgcttgtccac

SEQ ID NO: 435 - DNA, 17 bp, Homo sapiens
  source 1..17: mol_type = other DNA
ctatgtagctgatcttg

SEQ ID NO: 436 - DNA, 16 bp, synthetic construct
  source 1..16: mol_type = other DNA
  modified_base 1..3: mod_base = OTHER; note = locked nucleic acid
  modified_base 14..16: mod_base = OTHER; note = locked nucleic acid
  modified_base 1..16: mod_base = OTHER; note = phosphorothioate backbone
tagcactcgtagtaca

SEQ ID NO: 437 - DNA, 16 bp, synthetic construct
  source 1..16: mol_type = other DNA
  modified_base 1..3: mod_base = OTHER; note = locked nucleic acid
  modified_base 14..16: mod_base = OTHER; note = locked nucleic acid
  modified_base 1..16: mod_base = OTHER; note = phosphorothioate backbone
agcatggactcgtgca

SEQ ID NO: 438 - DNA, 16 bp, synthetic construct
  source 1..16: mol_type = other DNA
  modified_base 1..3: mod_base = OTHER; note = locked nucleic acid
  modified_base 14..16: mod_base = OTHER; note = locked nucleic acid
  modified_base 1..16: mod_base = OTHER; note = phosphorothioate backbone
agcatggactcgtgca

SEQ ID NO: 439 - DNA, 17 bp, synthetic construct
  source 1..17: mol_type = other DNA
  modified_base 1..3: mod_base = OTHER; note = locked nucleic acid
  modified_base 15..17: mod_base = OTHER; note = locked nucleic acid
  modified_base 1..17: mod_base = OTHER; note = phosphorothioate backbone
caagatcagctacatag

SEQ ID NO: 440 - DNA, 16 bp, synthetic construct
  source 1..16: mol_type = other DNA
tagcactcgtagtaca

SEQ ID NO: 441 - DNA, 16 bp, synthetic construct
  source 1..16: mol_type = other DNA
agcatggactcgtgca

SEQ ID NO: 442 - DNA, 16 bp, synthetic construct
  source 1..16: mol_type = other DNA
agcatggactcgtgca

SEQ ID NO: 443 - DNA, 17 bp, synthetic construct
  source 1..17: mol_type = other DNA
caagatcagctacatag